(19) United States
(12) Patent Application Publication
(10) Pub. No.: US 2024/0148500 A1
(43) Pub. Date: May 9, 2024

(54) FLEXIBLE CATHETER DEVICES AND METHODS OF MANUFACTURE AND USE

(71) Applicant: EDWARDS LIFESCIENCES CORPORATION, Irvine, CA (US)

(72) Inventors: Jose Luis Lopez, Fountain Valley, CA (US); Salomon Xavier Valencia, Aliso Viejo, CA (US); Brad Buzea, Trabuco Canyon, CA (US); Dang Khoa Ha Nguyen, Westminster, CA (US)

(21) Appl. No.: 18/415,323

(22) Filed: Jan. 17, 2024

Related U.S. Application Data
(63) Continuation of application No. PCT/US2021/058298, filed on Nov. 5, 2021.
(60) Provisional application No. 63/275,831, filed on Nov. 4, 2021.
(60) Provisional application No. 63/231,172, filed on Aug. 9, 2021.

(51) Int. Cl. A61F 2/24; A61M 25/00; A61M 25/01

(52) U.S. Cl. CPC A61F 2/243; A61M 25/0023; A61M 25/005; A61M 25/0147; A61M 2025/0004; A61M 2025/0063; A61M 2025/015

(57) ABSTRACT

A flexible delivery catheter comprising an outer jacket with a first portion and a second portion, a coil layer, and a coupler. The first portion and second portion of the outer jacket have a first inner diameter and a second inner diameter where the second inner diameter is greater than the first inner diameter. The change in diameter from the first inner diameter to the second inner diameter creates a shoulder. The coupler is coupled to the distal end of the outer jacket such that a cavity is formed by the coupler, the second portion of the outer jacket, and the shoulder of the outer jacket. A coil layer or hypotube is disposed within the cavity.

### RELATED APPLICATIONS

This application is a continuation of International Application No. PCT/US2021/058298, filed Nov. 5, 2021, which claims the benefit of U.S. Provisional Application No. 63/231,172, filed Aug. 9, 2021, and U.S. Provisional Application No. 63/275,831, filed Nov. 4, 2021, the contents of which are incorporated by reference in their entirety for all purposes.

### FIELD

The present application is directed to a sheath for use with catheter-based technologies for repairing and/or replacing heart valves, as well as for delivering an implant, such as a prosthetic valve to a heart via the patient's vasculature.

### BACKGROUND

Endovascular delivery catheter assemblies are used to implant prosthetic devices, such as a prosthetic valve, at locations inside the body that are not readily accessible by surgery or where access without invasive surgery is desirable. For example, aortic, mitral, tricuspid, and/or pulmonary prosthetic valves can be delivered to a treatment site using minimally invasive surgical techniques. Percutaneous interventional medical procedures utilize the large blood vessels of the body reach target destinations rather than surgically opening target site. There are many types of diseases states that can be treated via interventional methods including coronary blockages, valve replacements (TAVR) and brain aneurysms. These techniques involve using wires, catheters, balloons, electrodes and other thin devices to travel down the length of the blood vessels from the access site to the target site. The devices have a proximal end which the clinician controls outside of the body and a distal end inside the body which is responsible for treating the disease state. Percutaneous interventional procedures offer several advantages over open surgical techniques. First, they require smaller incision sites which reduces scarring and bleeding as well as infection risk. Procedures are also less traumatic to the tissue, so recovery times are reduced. Finally, interventional techniques can usually be performed much faster, and with fewer clinicians participating in the procedure, so overall costs are lowered. In some cases, the need for anesthesia is also eliminated, further speeding up the recovery process and reducing risk.

A single procedure typically uses several different guidewires, catheters, and balloons to achieve the desired effect. One at a time, each tool is inserted and then removed from the access site sequentially. For example, a guidewire is used to track to the correct location within the body. Next a balloon may be used to dilate a section of narrowed blood vessel. Last, an implant may be delivered to the target site. Because catheters are frequently inserted and removed, introducer sheaths are used to protect the local anatomy and simplify the procedure.

An introducer sheath can be used to safely introduce a delivery apparatus into a patient's vasculature (e.g., the femoral artery). Introducer sheaths are conduits that seal onto the access site blood vessel to reduce bleeding and trauma to the vessel caused by catheters with rough edges. An introducer sheath generally has an elongated sleeve that is inserted into the vasculature and a housing that contains one or more sealing valves that allow a delivery apparatus to be placed in fluid communication with the vasculature with minimal blood loss. Once the introducer sheath is positioned within the vasculature, the shaft of the delivery apparatus is advanced through the sheath and into the vasculature, carrying the prosthetic device. Expandable introducer sheaths, formed of highly elastomeric materials, allow for the dilating of the vessel to be performed by the passing prosthetic device. Expandable introducer sheaths are disclosed in U.S. Pat. No. 8,790,387, entitled “Expandable Sheath for Introducing an Endovascular Delivery Device into a Body,” U.S. Pat. No. 10,639,152, entitled “Expandable Sheath and Methods of Using the Same,” U.S. application Ser. No. 14/880,109, entitled “Expandable Sheath,” U.S. application Ser. No. 16/407,057, entitled “Expandable Sheath with Elastomeric Cross Sectional Portions,” U.S. Pat. No. 10,327,896, entitled “Expandable Sheath with Elastomeric Cross Sectional Portions,” U.S. application Ser. No. 15/997,587, entitled “Expandable Sheath for Introducing an Endovascular Delivery Device into a Body,” U.S. application Ser. No. 16/378,417, entitled “Expandable Sheath,” the disclosures of which are herein incorporated by reference.

Conventional methods of accessing a vessel, such as a femoral artery, prior to introducing the delivery system include dilating the vessel using multiple dilators or sheaths that progressively increase in diameter. Typically, the introducer is inserted into the sheath during preparation and both are then inserted into the vessel. Due to the need for a smooth transition from the introducer to the sheath, it is vital that the change in diameter of the introducer occurs distal to the tip of the sheath, such that the tip of the sheath fits snugly around the diameter. During insertion of the sheath and introducer, it is possible that the introducer can move backwards within the sheath, eliminating the aforementioned snug fit and creating a lip between the sheath tip and the smaller outer diameter of the introducer. This lip/gap can lead to severe vessel trauma during insertion.

Moreover, some procedures, such as a transseptal approach for mitral valve replacement/repair, require prolonged dilation of incisions in heart tissue and a curving/bending of the sheath to access the treatment site, prolonging procedure time and recovering and increasing risk of trauma to vessels and heart tissue.

Accordingly, there remains a need for further improvements in expandable introducer sheath for endovascular systems used to implant valves and other prosthetic devices.

### SUMMARY

Certain embodiments of the present disclosure provide a flexible delivery catheter for transporting a prosthetic into a patient via the human vasculature.

A flexible delivery catheter disclosed herein comprises an outer jacket, a coil layer, and a coupler. The outer jacket has a proximal end, a distal end, an inner surface and an outer surface, and defines a central lumen that extends longitudinally between the proximal end and the distal end. The coil layer is disposed at least partially within the central lumen of the outer jacket. The coil layer comprises a coil winding and has a proximal end, a distal end, an inner surface, and an outer surface. The coil winding extends helically about a longitudinal axis and defines a central lumen between the proximal end and the distal end. The coupler is provided at a distal end of the outer jacket, has a proximal end, a distal end, and an outer surface, and defines a central lumen that extends longitudinally between the proximal end and the distal end. The outer jacket includes a first portion with a first inner diameter and a second portion with a second inner diameter. The first portion is adjacent the proximal end of the outer jacket, and the second portion is adjacent the distal end of the outer jacket. The second inner diameter is greater than the first inner diameter. The outer jacket further includes a shoulder disposed between the first and second portions. The coupler and the second portion of the outer jacket form a cavity, and the coil layer is disposed within the cavity such that the coil layer abuts the inner surface of the second portion of outer jacket.

Another implementation of a flexible delivery catheter disclosed here comprises an outer jacket, a hypotube, and a coupler. The outer jacket has a proximal end, a distal end, an inner surface, and an outer surface, and defines a central lumen that extends longitudinally between the proximal end and the distal end. The hypotube is disposed at least partially within the central lumen of the outer jacket. The hypotube has a proximal end, a distal end, an inner surface, an outer surface, and a plurality of cut-outs which extend between the inner surface and the outer surface of the hypotube such that the hypotube can bend omnidirectionally. The coupler is provided at a distal end of the outer jacket. The coupler has a proximal end, a distal end, an outer surface, and defines a central lumen that extends longitudinally between the proximal end and the distal end. The outer jacket includes a first portion with a first inner diameter and a second portion with a second inner diameter. The first portion is adjacent the proximal end of the outer jacket, and the second portion is adjacent the distal end of the outer jacket. The second inner diameter is greater than the first inner diameter. The outer jacket further includes a shoulder disposed between the first and second portions. The coupler and the inner surface of the outer jacket (and the distal end of the inner liner when included) form a cavity. The hypotube is disposed within the cavity such that the hypotube abuts the inner surface of the second portion of the outer jacket.

A method of making a flexible delivery catheter disclosed herein comprises forming an outer jacket, forming a coil layer, and providing a coupler. The outer jacket has a proximal end, a distal end, an inner surface, an outer surface, a first portion with a first inner diameter, a second portion with a second inner diameter, and a shoulder between the first portion and the second portion. The outer jacket defines a central lumen that extends longitudinally between the proximal end and the distal end. The first portion is adjacent the proximal end of the outer jacket, and the second portion is adjacent the distal end of the outer jacket. The second inner diameter being greater than the first inner diameter. The coil layer comprises a coil winding and has a proximal end, a distal end, an inner surface, and an outer surface. The coil winding extends helically about a longitudinal axis and defines a central lumen between the proximal end and the distal end. The coupler has a proximal end, a distal end, and a radially extending protrusion and defines a central lumen extending longitudinally between the proximal end and the distal end. The coil layer is disposed within the central lumen of the outer jacket such that the coil layer abuts the inner surface of the second portion of the outer jacket and the shoulder of the outer jacket. The coupler is coupled to the distal end of the outer jacket such that the coupler abuts the distal end of the outer jacket and the coil layer is secured within in the second portion of the outer jacket between the proximal end of the coupler and the shoulder of the outer jacket.

Another method of making a flexible delivery catheter disclosed herein comprises forming an outer jacket, forming a hypotube, and providing a coupler. The outer jacket has a proximal end, a distal end, an inner surface, an outer surface, a first portion with a first inner diameter, a second portion with a second inner diameter, and a shoulder disposed between the first portion and the second portion. The outer jacket defines a central lumen that extends longitudinally between the proximal end and the distal end. The first portion is adjacent the proximal end of the outer jacket, and the second portion is adjacent the distal end of the outer jacket. The second inner diameter is greater than the first inner diameter. The hypotube has a proximal end, a distal end, an inner surface, an outer surface, and a plurality of cut-outs which extend between the inner surface and the outer surface of the hypotube such that the hypotube can bend omnidirectionally. The coupler has a proximal end, a distal end, and a radially extending protrusion. The coupler defines a central lumen that extends longitudinally between the proximal end and the distal end. The hypotube is disposed within the central lumen of the outer jacket such that the hypotube abuts the inner surface of the second portion of the outer jacket and the shoulder of the outer jacket. The coupler is coupled to the distal end of the outer jacket such that the coupler abuts the distal end of the outer jacket and the hypotube is secured within the second portion of the outer jacket between the proximal end of the coupler and the shoulder of the outer jacket.

A method of deploying a prosthetic into a patient disclosed herein comprises providing a delivery system comprising a flexible catheter having a proximal end and a distal end, advancing the catheter into a vessel of a patient, advancing the prosthetic from the proximal end of the catheter to the distal end of the catheter and into the patient.

Another implementation of a flexible delivery catheter disclosed here comprises an outer jacket, having a proximal end, a distal end, an inner surface and an outer surface, and defining a central lumen extending longitudinally between the proximal end and the distal end, the outer jacket defining a flexible portion extending along a length of the outer jacket proximal the distal end; a pullwire mechanism coupled to the outer jacket for directing bending motion of the flexible portion of the outer jacket. The pullwire mechanism including: a pullwire extending along the outer jacket and coupled to the outer jacket at a location proximate the distal end of the outer jacket, and a knob coupled to the pullwire such that rotational movement of the knob causes the pullwire to move in a proximal or distal direction resulting in a corresponding bending motion of the flexible portion of the outer jacket; and a valve deployment assembly coupled to the outer jacket for directing proximal and distal movement of the outer jacket. The valve deployment assembly including: a housing with a central lumen extending therethrough, the central lumen including a threaded inner surface, an inner member extending within the central lumen of the housing, the inner member rotatably coupled to the housing and including a central lumen extending therethrough, a coupler fixedly coupled to the outer jacket and rotatably coupled to the housing, the coupler extending within the central lumen of the housing and the inner member, a proximal end portion of the coupler including a threaded outer surface that threadingly engages the threaded inner surface of the housing. Rotational movement of the housing causes the threaded inner surface of the housing to engage the threaded outer surface of the coupler resulting in a corresponding axial movement of the coupler within the housing and corresponding axial movement of the outer jacket in the proximal or distal direction.

### DESCRIPTION OF DRAWINGS

FIG. 1 is a side view of the heart valve delivery system delivering a heart valve to a native valve site according to one example of the present disclosure.

FIG. 2 is a perspective view of delivery catheter (delivery sheath) of FIG. 1.

FIG. 3 is a partial side cross section view (not to scale) of an example flexible catheter including a coil layer and a braided layer extending along the entirety (or a portion) of the length of the catheter.

FIG. 4 is a partial side cross section view (not to scale) of an example flexible catheter including a coil layer and a braided layer.

FIG. 5 is a partial side cross section view (not to scale) of an example flexible catheter including a coil layer, a braided layer, and a PET layer surrounding the coil layer.

FIG. 6 is a partial side cross section view (not to scale) of an example flexible catheter having a variable density braided layer.

FIG. 7 is a partial side cross section view (not to scale) of an example flexible catheter having a braided layer.

FIG. 8 is a partial side cross section view (not to scale) of an example flexible catheter that includes a hypotube.

FIG. 9 is a partial side cross section view (not to scale) of an example flexible catheter that includes a hypotube.

FIG. 10 is a side view of the example hypotube of FIG. 8 or FIG. 9.

FIG. 11 is a finite element analysis of the stress experience by the example hypotube of FIG. 10 during bending.

FIG. 12 a flattened view of the hypotube shown in FIG. 8 or FIG. 9.

FIG. 13 is a side and end view of an example hypotube.

FIG. 14A is a partial side cross section view of an example flexible catheter including a coil layer and a braided layer.

FIG. 14B is a perspective view of the example flexible catheter of FIG. 14A.

FIG. 15A is a proximal end view of the coupler of FIGS. 14A and 14B.

FIG. 15B is a first elevation view of the coupler of FIGS. 14A and 14B.

FIG. 15C is a distal end view of the coupler of FIGS. 14A and 14B.

FIG. 15D is a first perspective view of the coupler of FIGS. 14A and 14B.

FIG. 15E is a second elevation view of the coupler of FIGS. 14A and 14B.

FIG. 15F is a second perspective view of the coupler of FIGS. 14A and 14B.

FIG. 15G is cross section view of the coupler of FIGS. 14A and 14B.

FIG. 16A is a partial side view of an example flexible catheter including a coil layer and a ring.

FIG. 16B is a partial side view of the flexible catheter of FIG. 16A with a transparent outer jacket.

FIG. 17 is a perspective view of the ring of FIGS. 16A and 16B.

FIG. 18A is cross section perspective view of an example flexible catheter including a coil layer and a ring.

FIG. 18B is a partial perspective view of the flexible catheter of FIG. 18A with a transparent outer jacket.

FIG. 19 is a perspective view of the ring of FIGS. 18A and 18B.

FIG. 20A is a partial side cross section view of an example flexible catheter including a hypotube and a braided layer.

FIG. 20B is a perspective view of the flexible catheter of FIG. 20A.

FIG. 21 is a perspective view of the hypotube of FIGS. 20A and 20B.

FIG. 22A is a proximal end view of the coupler of FIGS. 20A and 20B.

FIG. 22B is a first elevation view of the coupler of FIGS. 20A and 20B.

FIG. 22C is a distal end view of the coupler of FIGS. 20A and 20B.

FIG. 22D is a first perspective view of the coupler of FIGS. 20A and 20B.

FIG. 22E is a second elevation view of the coupler of FIGS. 20A and 20B.

FIG. 22F is a second perspective view of the slider of FIGS. 20A and 20B.

FIG. 22G is cross section view of the coupler of FIGS. 20A and 20B.

FIG. 23A is a partial side cross section view of an example flexible catheter a hypotube and a ring.

FIG. 23B is a partial side view of the flexible catheter of FIG. 23A with a transparent outer jacket.

FIG. 24 is a perspective view of the ring of FIGS. 23A and 23B.

FIG. 25 is a perspective view an example flexible catheter including a pullwire mechanism and a valve deployment assembly.

FIG. 26 is a cross section view of the flexible catheter of FIG. 25, with the valve deployment assembly and the pullwire mechanism in a distal position.

FIG. 27 is a partial cross section view of the flexible catheter of FIG. 25, with the valve deployment assembly and the pullwire mechanism is a proximal position.

FIG. 28 is a partial perspective view of the flexible delivery catheter of FIG. 25 with the knob removed.

FIG. 29A is a left side view of the example knob of FIG. 25.

FIG. 29B is a front view of the example knob.

FIG. 29C is a right side view of the example knob.

FIG. 29D is a cross section view of the example knob taken along section line A-A in FIG. 29B.

FIG. 29E is a perspective view of the example knob

FIG. 30A is a top view of the example coupler of FIG. 25.

FIG. 30B is a front view of the example coupler.

FIG. 30C is a left side view of the example coupler.

FIG. 30D is a right side view of the example coupler.

FIG. 30E is a perspective view of the example coupler.

FIG. 31A is a left side view of the example threaded sleeve of FIG. 25.

FIG. 31B is a top view of the example threaded sleeve.

FIG. 31C is a front view of the example threaded sleeve.

FIG. 31D is a bottom view of the example threaded sleeve.

FIG. 31E is a right side view of the example threaded sleeve.

FIG. 31F is a perspective view of the example threaded sleeve.

FIG. 32A is top view of the example anchor sleeve of FIG. 25.

FIG. 32B is a front view of the example anchor sleeve.

FIG. 32C is a right side view of the example anchor sleeve.

FIG. 32D is a perspective view of the example anchor sleeve.

FIG. 33A is a left side view of the example housing of FIG. 25.

FIG. 33B is a front view of the example housing.

FIG. 33C is a right side view of the example housing.

FIG. 33D is a cross section view of the example housing taken along section line A-A in FIG. 33B.

FIG. 33E is a perspective view of the example housing.

FIG. 34A is a left side view of the example inner member of FIG. 25.

FIG. 34B is a front view of the example inner member.

FIG. 34C is a bottom view of the example inner member.

FIG. 34D is a back view of the example inner member.

FIG. 34E is a top view of the example inner member.

FIG. 34F is a right side view of the example inner member.

FIG. 34G is a top perspective view of the example inner member.

FIG. 34H is a front perspective view of the example inner member.

FIG. 35 is a perspective view of the delivery catheter of FIG. 25 including a delivery capsule coupled to the distal end of the outer jacket.

FIG. 36 is a partial cross section view of the distal end of the delivery catheter and the delivery capsule of FIG. 35.

FIG. 37 is a partial perspective view of the delivery catheter and the delivery capsule of FIG. 35.

FIG. 38 is a partial perspective cross section view of the delivery catheter and the delivery capsule of FIG. 35.

FIG. 39 is an exploded view of the delivery catheter of FIG. 35.

FIG. 40 is a partial cross section view of the distal end of the delivery catheter of FIG. 35.

FIG. 41 illustrates the delivery capsule withdrawn from a prosthetic device.

### DETAILED DESCRIPTION

The following description of certain examples of the inventive concepts should not be used to limit the scope of the claims. Other examples, features, aspects, embodiments, and advantages will become apparent to those skilled in the art from the following description. As will be realized, the device and/or methods are capable of other different and obvious aspects, all without departing from the spirit of the inventive concepts. Accordingly, the drawings and descriptions should be regarded as illustrative in nature and not restrictive.

For purposes of this description, certain aspects, advantages, and novel features of the examples of this disclosure are described herein. The described methods, systems, and apparatus should not be construed as limiting in any way. Instead, the present disclosure is directed toward all novel and nonobvious features and aspects of the various disclosed examples, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Features, integers, characteristics, compounds, chemical moieties, or groups described in conjunction with a particular aspect, embodiment or example of the invention are to be understood to be applicable to any other aspect, embodiment or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The invention is not restricted to the details of any foregoing examples. The invention extends to any novel one, or any novel combination, of the features disclosed in this specification (including any accompanying claims, abstract, and drawings), or to any novel one, or any novel combination, of the steps of any method or process so disclosed.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

The terms “proximal” and “distal” as used herein refer to regions of a sheath, catheter, or delivery assembly. “Proximal” means that region closest to handle of the device, while “distal” means that region farthest away from the handle of the device.

“Axially” or “axial” as used herein refers to a direction along the longitudinal axis of the sheath.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

The term “tube” or “tubular” as used herein is not meant to limit shapes to circular cross-sections. Instead, tube or tubular can refer to any elongate structure with a closed-cross section and lumen extending axially therethrough. A tube may also have some selectively located slits or openings therein—although it still will provide enough of a closed structure to contain other components within its lumen(s).

### Sheath

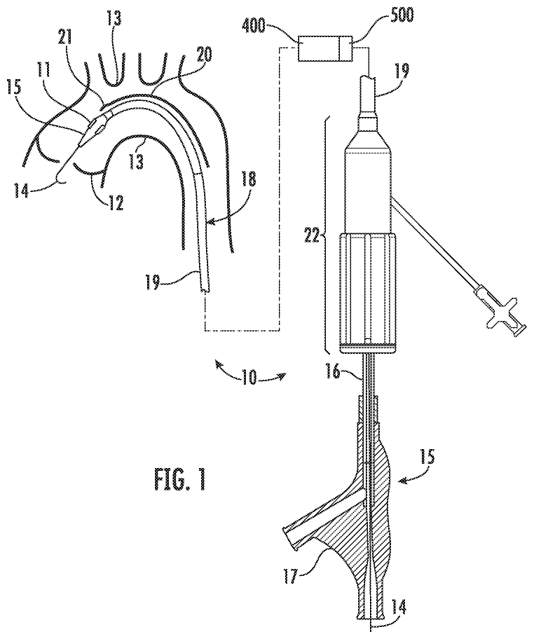

An exemplary delivery system is shown in FIG. 1. FIG. 1, for purposes of illustration, shows an example of a heart valve delivery system 10 for delivering a prosthetic valve 11 to a diseased aortic valve 12 of a human heart is shown. The delivery system is well-suited for delivering the prosthetic valve 11 through a patient's vasculature and over an aortic arch 13 to a location adjacent to the diseased aortic valve 12.

The delivery system 10 generally includes a guidewire 14 and a balloon catheter 15 configured for advancement over the guidewire 14. The prosthetic valve 11 is provided along the distal end portion of the balloon catheter. The balloon catheter 15 includes a tubular section 16 and a handle/support 17 at a proximal end of the tubular section 16. The tubular section 16 of the balloon catheter 15 is received within a delivery sleeve assembly 18. The delivery sleeve assembly generally comprises an elongated polymeric sleeve 19 as disclosed in detail herein, a steerable and/or flexible distal tip section 20, and a coupler 21. A proximal end of the delivery sleeve assembly 18 is mounted to a handle 22. The delivery system 10 passes through an introducer sheath assembly 400 and a loader assembly 500, both of which will be described in more detail below, to enter the body vessel and deliver the prosthetic valve 11.

The following figures demonstrate described herein examples in more detail. Additional examples can also be found in U.S. Pat. Nos. 7,780,723, 8,382,826, 9,028,545, 9,907,651, and 10,478,294, contents of which are incorporated herein by reference in their whole entirety.

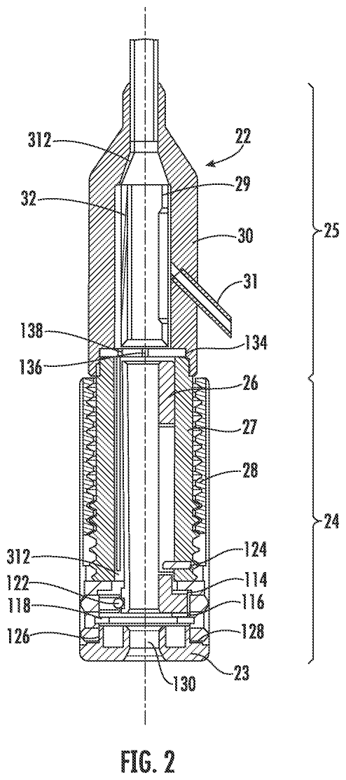

With reference to FIG. 2, the handle 22 at the proximal end of the delivery sleeve assembly 18 generally includes an end cap 23, an adjustable portion 24, and a hemostasis portion 25. The adjustable portion 24 includes a first core member 26, a partially threaded member 27 around the first core member 26, and a rotator handle 28 around the partially threaded member 27. The hemostasis portion 25 includes a second core member 29 and a hub 30 around the second core member 29. A hemostasis tube 31 extends outwards from the hub 30. A guide tube 32 is placed within the handle 22, as will be described in greater detail below.

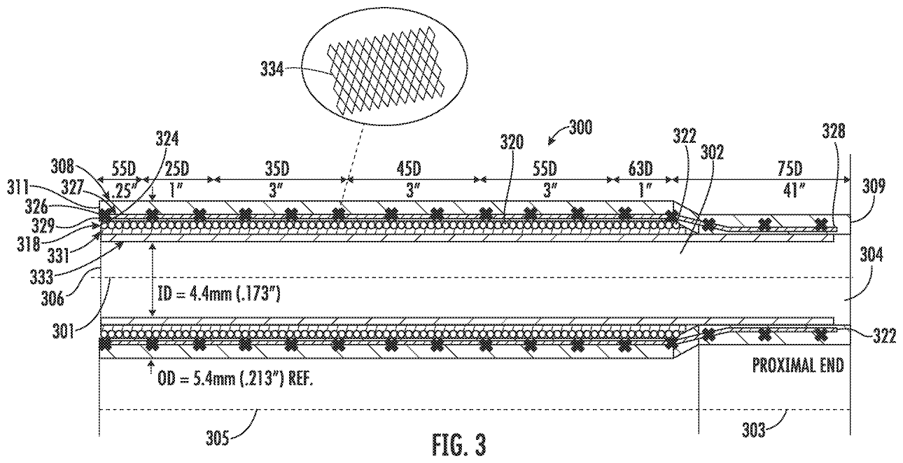

FIG. 3 shows a flexible introducer catheter, i.e., delivery catheter 300. The delivery catheter 300 can provide an omni-directional flexible catheter which is bendable in any direction away from a longitudinal axis 301 of the delivery catheter 300 while retaining compressive and tensile stiffness. As such, the flexible delivery catheter 300 conforms to a patient's vascular structure during placement and delivering a medical device (e.g., prosthetic heart valve), while limiting axial deformation of the delivery catheter 300 that can damage a patient's vascular structure. As will be described in more detail below, the delivery catheter 300 includes, a coil layer 318 which provides compressive stiffness to the delivery catheter 300, a braided layer 326 which provides structural rigidity, a tensile stiffening fiber 327 which provides tensile stiffness, and an outer jacket 308 having various elasticity characteristics along its axial length. The delivery catheter 300 defines an elongated tubular structure with a central lumen 302 extending longitudinally between the proximal end 304 and the distal end 306. The central lumen 302 (and in each example described below) is configured for passage of medical devices such as prosthetic heart valves therethrough. The delivery catheter 300 includes an elongated body portion 303 and a distal tip portion 305. The elongated body portion 303 extends from the distal tip portion 305 (at the distal end 306 of the delivery catheter 300) to the proximal end 304 of the delivery catheter 300. The elongated body portion 303 provides a section of the delivery catheter 300 that is rigid enough for effective manipulation by a physician (e.g., advancement through the patient's vasculature). The distal tip portion 305 has a less rigid structure than the elongated body portion 303, which is suitable for conforming to the vascular structure of a patient during a delivery procedure, as described above.

As illustrated in FIG. 3, the coil layer 318 and the braided layer 326 are provided at the distal tip portion 305 of the delivery catheter 300. The coil layer 318 is disposed within the inner lumen of the braided layer 326. The braided layer 326 is disposed between the outer jacket 308 and the coil layer 318. The coil layer 318 and the braided layer 326 are provided at the distal tip portion 305 of the delivery catheter 300. As such, the coil layer 318 is wound to resist axial compression and tension applied to the delivery catheter 300, the coil layer 318 facilitates bending of the delivery catheter 300 at the distal tip portion 305 in a direction away from the longitudinal axis 301 of the delivery catheter 300.

As illustrated in FIG. (Concept 2), the distal tip portion 305 of the delivery catheter 300 has an outer diameter that is greater than an outer diameter of the elongated body portion 303, whereas the inner diameter of the delivery catheter 300, along both the elongated body portion 303 and the distal tip portion 305, remains constant. The outer surface of the distal tip portion 305 adjacent the body portion 303 includes a tapered surface extending toward the outer surface of the body portion 303. This decreasing taper is provided between the larger outer diameter distal tip portion 305 and the reduced diameter body portion 303.

In the example shown in FIG. 3, the outer diameter of the body portion 303 is about 5.4 mm But in other examples the outer diameter of the body portion 303 ranges between about 4.5 mm and about 6.0 mm or between about 5.0 mm and about 6.0 mm. In the example shown in FIG. 3, the outer diameter of the distal tip portion 305 is about 5.59 mm. In other examples, the outer diameter of the distal tip portion 305 ranges between about 4.5 mm and about 6.0 mm, or between about 5.0 mm and about 6.0 mm. In the example shown in FIG. 3, the inner diameter of the central lumen 302 of the delivery catheter 300 is about 4.4 mm. In other examples, the inner diameter of the central lumen 302 of the delivery catheter 300 ranges between 3.5 mm and about 5.0 mm or between 4.0 mm and about 4.5 mm.

The distal tip portion 305 extends along a length of the delivery catheter 300 from a distal end 306 of the delivery catheter 300 toward the proximal end 304 of the delivery catheter 300. In the example shown in FIG. 3, the length of the distal tip portion 305, which is measured in a direction along the longitudinal axis of the delivery catheter 300 is about 285.75 mm (11.25 inches). In some examples, the distal tip portion 305 has a length less than 508 mm (20 inches), less than 381 mm (15 inches), between 127 mm (5 inches) and 381 mm (15 inches), or between 254 mm (10 inches) and 381 mm (15 inches). In the example shown in FIG. 3, the length of the body portion 303 is about 1041.4 mm (41 inches). In some examples, the length of the body portion 303 ranges between about 762 mm (30 inches) to about 1270 mm (50 inches) or between about 889 mm (35 inches) to about 1143 mm (45 inches).

As described above, the delivery catheter 300 includes a coil layer 318. The coil layer 318 is wound to resist axial compression applied to the delivery catheter 300, while facilitating bending of the delivery catheter 300 in a direction away from the longitudinal axis of the delivery catheter 300. The coil layer 318 includes a coil winding 320 which provides compressive stiffness to the delivery catheter 300 during axial compressive loads. The coil layer 318 has a proximal end 322, a distal end 324, an inner surface, and an outer surface. The coil winding 320 extends helically about a longitudinal axis 321 and defines a central lumen 323 between the proximal end 322 and the distal end 324. In the example shown in FIG. 3, the coil layer 318 has a plurality of tightly wound turns of the coil winding 320. In an example coil layer 318, a gap/spacing is provided between adjacent turns of the coil winding 320. In another example, the coil layer 318 is tightly wound such that the adjacent turns of the coil winding 320 contact, i.e., an outer surface of the adjacent turns of the coil winding 320 contact along a circumferential length of the coil winding 320. This allows for some axial compression in certain applications that require partial axial compression of the catheter. Adjacent at the ends of the coil layer 318 are welded together to control axial expansion and placement of the coil layer 318 during manufacturing. In the example shown in FIG. 3, the coil layer 318 has a length that is less than a length of the distal tip portion 305. But in other examples, a length of the coil layer 318 is greater than a length of the distal tip portion 305 such that the coil layer 318 extends into the body portion 303 of the delivery catheter 300.

In the example shown in FIG. 3, the coil winding 320 has a diameter ranging between about 0.003 inches and about 0.010 inches, and between about 0.004 inches and about 0.008 inches. In one example the coil winding 320 has a diameter of about 0.004 inches. In other examples, the coil winding 320 has a diameter of about 0.008 inches.

As described above, the coil winding 320 has a constant pitch along the axial length of the coil layer 318. But in other implementations, the coil winding 320 has a varying pitch along an entire length of the coil winding 320/coil layer 318. For example, a pitch of the coil winding 320 at a proximal end of the distal tip portion 305, e.g., along the tapered portion of the distal tip portion 305, can be less than a pitch of the coil winding 320 at the distal end 306 of the distal tip portion 305. In the example shown in FIG. 3 the coil layer 318 has about 55 turns per inch, but in other examples, the coil has between about 50 and 120 turns per inch, in implementations having 0.008 inch diameter coil wire.

The coil layer 318 can be composed of at least one of a polymer, a metal, or a composite. For example, the coil layer 318 can be composed of a stainless steel wire. The coil winding 320 has a curvilinear shape in cross section, but the coil winding 320 can have either a rectilinear or curvilinear shape in cross section.

As described above the delivery catheter 300 includes a braided layer 326 which provides structural support for the delivery catheter 300. The braided layer 326 has a proximal end 330 and a distal end 332. The braided layer 326 includes a plurality of fibers 334 interwoven into a braided pattern and defines a central lumen, an inner surface 338, and an outer surface 340. In the example shown in FIG. 3, the braided layer 326 extends along the entire length of the body portion 303 and the distal tip portion 305 of the delivery catheter 300. In another example, the braided layer 326 extends along an entire length of the distal tip portion 305. The braided layer 326 extends along at least a portion of the entire length of the body portion 303 of the delivery catheter 300. In the example shown in FIG. 3 the braided layer 326 extends along the entire length of the delivery catheter 300. As such, braided layer 326 has a length that is greater than the overall length of the coil layer 318. But, in some implementations the braided layer 326 and the coil layer 318 are about the same length along the axis of the delivery catheter 300.

As described above, the braided layer 326 is composed of a plurality of interwoven fibers 334. Each of the fibers 334 has a curvilinear cross section, although in other examples, the fibers 334 have a rectilinear cross section. The fibers 334 are composed of stainless steel, but in other examples, the fibers 334 are composed of at least one of a polyester, a polymer, a metal, a composite or any other material suitable for composing a stiffening braided layer 326 in a delivery catheter 300. Each of the plurality of interwoven fibers 334 has a diameter of about 0.004 inches. In other examples, each of the plurality of interwoven fibers 334 has a diameter ranging between about 0.002 inches and about 0.006 inches or between about 0.003 inches and about 0.005 inches. The interwoven fibers 334 can be oriented in a single strand braid pattern or in a double strand braid pattern where each interweave includes a plurality of parallel fibers 334 for each weave strand. The plurality of interwoven fibers 334 is composed of 16 individual fibers 334. But in other examples the plurality of interwoven fibers 334 is composed of about 10 to about 20 individual fibers 334. The braided layer 326 has a braid density which allows for omnidirectional bending with respect to the central axis of the delivery catheter 300. As the braid density increases, the braided layer 326 becomes more resistant to bending. In the example shown in FIG. 3, the braid density of the braided layer 326 is about 25 picks per inch. But in other examples, the braided layer 326 has a braid density between about 20 to about 30 picks per inch.

As described above, the delivery catheter 300 as shown in FIG. 3 includes at least one tensile stiffening fiber 327 having a proximal end 328 and a distal end 329. The tensile stiffening fiber 327 limits tensile deformation along the axial length of the delivery catheter 300 such that the bending of the delivery catheter 300 is limited at a location/along the side corresponding to the circumferential location of the tensile stiffening fiber 327. For example, if the tensile stiffening fiber is located at a 90-degree position (e.g., 3 o'clock) around the circumference of the delivery catheter 300, the delivery catheter 300 will resist bending at the circumferentially 90-degree position (e.g., 3 o'clock). Generally, the location of the tensile stiffening fiber 327 determines the neutral bending axis of the delivery catheter 300. That is, the stiffness and relative location of the tensile stiffening fiber 327 (or location of multiple fibers with respect to each other) defines the line or plane along the delivery catheter 300 at which no extension or compression occurs when the catheter is bent. For example, when the delivery catheter 300 includes a single tensile stiffening fiber 327, the neutral axis of the delivery catheter is defined by the axis of the tensile stiffening fiber 327.

The tensile stiffening fiber 327 extends along at least a portion of the entire length of the body portion 303 of the delivery catheter 300. In the example shown in FIG. 3 the least one tensile stiffening fiber 327 extends along an entire length the body portion 303 of the delivery catheter 300. But, in other examples, a length of the at least one tensile stiffening fiber 327 corresponds to a length of the distal tip portion 305 or is greater than the length of the distal tip portion 305.

The tensile stiffening fiber 327 is coupled to the delivery catheter 300 by the weaving/intertwining the tensile stiffening fiber 327 within the fibers/braid pattern of the braided layer 326. The tensile stiffening fiber 327 is further secured to the braided layer 326 when the outer jacket 308 is reflowed into the braided layer 326 during manufacturing. As will be described in more detail below, the distal end of the delivery catheter 300 includes materials of varying durometer along the length of the outer jacket 308. A length/segment of the delivery catheter 300 proximate the distal end 306 of the distal tip portion 305 comprises a material having a higher durometer than the next adjacent segment of the delivery catheter 300. Reflow of the tensile stiffening fiber 327 with the higher durometer material at the distal end 306 of the distal tip portion 305, results in higher friction between the tensile stiffening fiber 327 and the higher durometer material of the outer jacket 308, thereby preventing axial slippage/movement of the tensile stiffening fiber 327 at the distal end of the catheter. Similarly, the proximal end of the delivery catheter 300 includes a length/segment of material having a higher durometer than the next adjacent segment of the delivery catheter 300. Accordingly, reflow of the tensile stiffening fiber 327 with the higher durometer material at the proximal end 304 of the delivery catheter 300 prevents slippage/movement of the tensile stiffening fiber 327 with respect to the end of the delivery catheter 300. As such, the tensile stiffening fiber 327 is secured both along the length of the fiber and at its proximal and distal ends, i.e., the tensile stiffening fiber 327 is fixedly coupled to the proximal and distal ends of the outer jacket 308/braided layer 326 and along the along the braided layer 326.

As shown in FIG. 3, the tensile stiffening fiber 327 is woven into the braided layer 326. In the example shown in FIG. 3, the at least one tensile stiffening fiber 327 is woven into the braided layer 326 along an entire length of the braided layer 326. But, in other examples, it is contemplated that the tensile stiffening fiber 327 is woven into a portion of the length of the braided layer 326. For example, the at least one tensile stiffening fiber 327 may not be woven into a first portion of the braided layer 326 extending along the distal tip portion 305 of the delivery catheter 300, but will be woven into a second portion of the braided layer 326 extending along an elongated body portion 303 of the delivery catheter 300 between the distal tip portion 305 and the proximal end 304 of the delivery catheter 300.

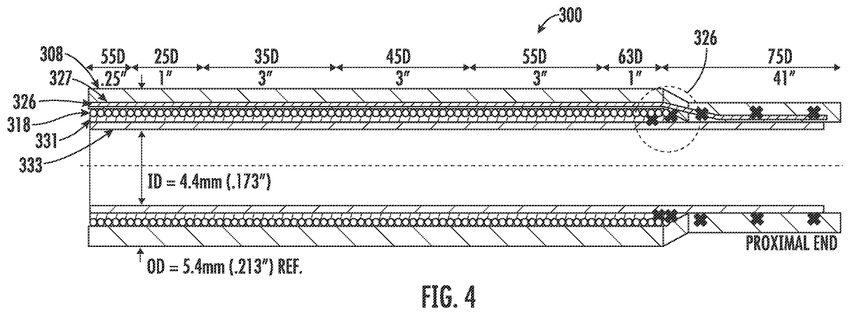

The example shown in FIG. 3 and FIG. 4 includes Technora® Kevlar. But other examples include a tensile stiffening fiber 327 composed of at least one of a polymer, a metal, a composite including a polyamide type polymer, an other Kevlar or Liquid Crystal Polymer (LCP).

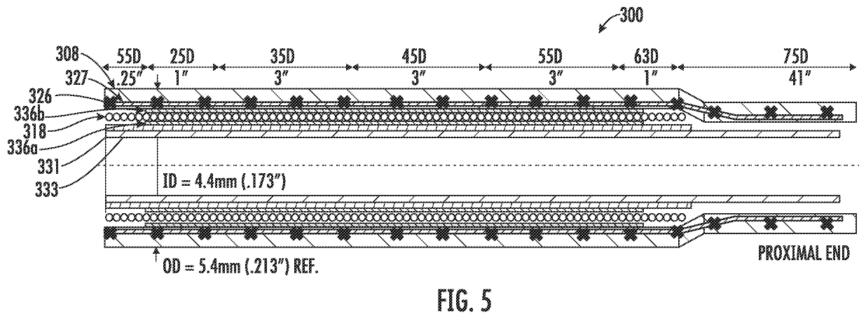

The example shown in FIG. 3 and FIG. 5 include four tensile stiffening fibers 327 spaced around the circumference of the delivery catheter 300 by 90 degrees. But other examples include six or less, four or less, two or less, or one individual tensile stiffening fiber 327. In examples having a plurality of tensile stiffening fibers 327, the plurality of individual tensile stiffening fibers 327 can be equally spaced around the circumference of the delivery catheter 300 (e.g., every 60 degrees, 90 degrees, 120 degrees, 180 degrees), or irregularly spaced around the circumference of the delivery catheter 300. In examples having a one individual tensile stiffening fiber 327, the one individual tensile stiffening fiber 327 is located at a neutral axis of the delivery catheter 300.

As described above, the delivery catheter 300 includes an outer jacket 308, having a proximal end 309 and a distal end 311 which forms the outermost layer of the delivery catheter 300. The outer jacket 308 is composed of an elastic material. The elasticity of the outer jacket 308 varies (longitudinally) along a length of the outer jacket 308. In the example shown in FIG. 3 and FIG. 5, the elasticity of the outer jacket 308 increases between the proximal and distal ends 309, 311 of the outer jacket 308 such that the distal end 306 (e.g., distal tip portion 305) of the delivery catheter 300 more easily bends to conform to the aortic geometry of a patient. The durometer of the outer jacket 308 decreases between the proximal end 304 and the distal end 306 of the catheter 300. However, as described above, the distal most and proximal most segments of the delivery catheter 300 is constructed from a material having a higher durometer (and lower elasticity) than the next adjacent segment to facilitate coupling of the tensile stiffening fiber 327 to the proximal and distal ends 309, 311 of the delivery catheter 300. The outer jacket 308 includes various longitudinal segments which are constructed from materials having varying durometer. The materials of the various longitudinal segments are reflowed together to form a single outer jacket 308. In the example delivery catheter 300 illustrated in FIGS. 3 and 5, the delivery catheter 300 is divided into various length segments. The distal tip portion 305 includes seven segments of various lengths and durometer material. For example, the first/distal most segment is composed of a material having a durometer of 55D and a length of about 0.25 inches. The second segment is composed of material having a durometer of 25D and a length of about 1 inch. The third segment is composed of material having a durometer of 35D and a length of about 3 inches. The fourth segment is composed of material having a durometer of 45D and a length of about 3 inches. The fifth segment is composed of material having a durometer of 63D and a length of about 1 inch. The sixth/proximal most segment (along the body portion 303 of the delivery catheter 300) is composed of a material having a durometer of 75D and a length of about 41 inches. It is contemplated that the number, length and durometer of the various segments may vary from those illustrated and described above. As mentioned above, the distal most segment (referred to also as a coupling segment) and the proximal most segment (on the body portion 303) have a lower elasticity and higher durometer than the next adjacent segment of the outer jacket 308 to help secure the tensile stiffening fiber 327 to the outer jacket 308 post-reflow procedure.

In the example shown in FIG. 3 the outer jacket 308 is composed of Pebax. But, in other examples, at least a portion of the outer jacket 308 comprises another polyether block amide, another elastomer, a polyamide (e.g., Vestamid®), or any other material suitable for forming the outermost layer of a flexible catheter.

In addition to the components described above, the delivery catheter 300 in the examples shown in FIGS. 3-5 include a tie layer 331. The tie layer 331 is provided on an inner surface of the coil layer 318 and creates a surface for improved adhesion between the coil layer 318 and the liner 333 which is described below. The tie layer 331 bonds the liner 333 to the outer jacket 308. The tie layer 331 is reflowed and/or bonded with the outer jacket 308. The tie layer does not bond to the coil layer 318, instead it encapsulates the coil layer 318 preventing material from entering between the coil windings. As illustrated in FIG. 3, the tie layer 331 extends along the entire axial length of the inner surface of the coil layer 318. In other examples as shown in FIG. 5, the tie layer 331 extends along a portion of the axial length of the coil layer 318. The tie layer 331 has a thickness of about 0.003 inches, but in other examples the tie layer 331 has a thickness ranging between about 0.002 inches and about 0.005 inches. The tie layer 331 is composed of a polyether block amide material such as Pebax, and/or a polyamide (e.g., Vestamid®), polyamide 12 (e.g., Rilsamid®), or any other suitable material.

As described above, the delivery catheter 300 includes a liner 333 provided on an inner surface of the tie layer 331. An inner surface of the liner 333 defines an inner lumen of the delivery catheter 300. The liner 333 extends along an entire length of the inner surface of the tie layer 331 and/or the inner lumen of the delivery catheter 300. In some examples, the liner 333 has a length that is greater than the length of the distal tip portion 305. In some examples, the liner 333 extends along at a portion of the entire length of the body portion 303 of the delivery catheter 300 or the entire length of the body portion 303 of the delivery catheter 300. The liner 333 in the example shown in FIG. 3 has a thickness of about 0.002 inches. But in other examples, the liner 333 has a thickness ranging between 0.001 inches and about 0.004 inches or between 0.0015 inches and about 0.003 inches. The liner 333 is composed of polytetrafluoroethylene (PTFE), Polyamide 12 materials, polyether block amide (PEBA), fluorinated ethylene propylene (FEP), or any other polymer suitable for forming an inner most layer of a flexible delivery catheter 300.

The example shown in FIG. 5 includes a barrier layers 336a-b formed from polythioethylene (PET). In other examples, the barrier layers are formed from other polymers. The flexible delivery catheter 300 in the example shown in FIG. 5 the PET layer forms an outer barrier layer 336b between an outer surface 317 of the coil layer 318 and the inner surface of the outer jacket 308/braided layer 326. An inner barrier layer 336a provided between the inner surface of the coil layer 318 and the tie layer 331. The inner and outer barrier layers 336a, 336b extend along a majority of the length of the coil layer 318. The inner and outer barrier layers 336a, 336b are not provided along a distal length of the coil layer 318, e.g., a length extending proximally from the distal end 324 of the coil layer 318. Accordingly, the distal end 324 of the coil layer 318 is fixedly coupled at the distal end 306 of the delivery catheter 300 when the outer jacket 308 bonded (e.g., reflowed) to the tie layer 331. Similarly, the inner and outer barrier layers 336a, 336b are not provided along a length of the coil layer 318 extending distally from the proximal end 322 of the coil layer 318 such that the proximal end 322 of the coil layer 318 is fixedly attached to the outer jacket 308. Like the distal end 324, the proximal end 322 of the coil layer 318 is fixedly coupled to the outer jacket 308 when the outer jacket 308 is bonded (e.g., reflowed) to the tie layer 331. In the example shown in FIG. 5 the inner and outer barrier layers have a thickness of about 0.005 inches. But, in other examples the inner and outer barrier layers have a thickness ranging between about 0.0003 inches and about 0.0007 inches or between about 0.0004 inches and about 0.0006 inches.

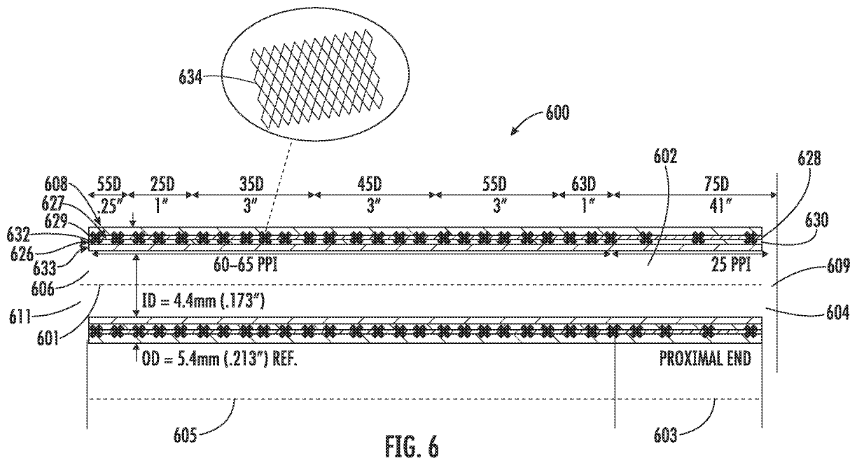

FIG. 6 shows another example of the flexible delivery catheter 600 having a variable braid density braided layer 626. The delivery catheter 600 includes similar structures and features to delivery catheter 300 described above, the differences will be discussed in more detail below, like element numbers will be used to identify like elements. The delivery catheter 600 has a proximal end 604 and a distal end 606, an inner surface, and an outer surface 610 and includes an outer jacket 608, having a proximal end 609, a distal end 611. The delivery catheter 600 defines a central lumen 602 extending longitudinally between the proximal end 604 and the distal end 606. The braided layer 626 is embedded in the outer jacket 608 to provide structural rigidity. The braided layer 626 has a plurality of interwoven fibers disposed in a braided pattern forming an elongated tubular structure defining a central lumen 602 extending therethrough. A braid density of the braided pattern varies along a length of the braided layer 626 as described above. The delivery catheter 600 is omnidirectionally flexible and configured to bend in any direction away from a longitudinal axis 601 of the delivery catheter 600 while retaining compressive and tensile stiffness. As such, the flexible delivery catheter 600 conforms to a patient's vascular structure and during placement and delivery of a medical device (e.g., prosthetic heart valve), while limiting axial deformation of the delivery catheter 600 that can damage a patient's vascular structure.

The delivery catheter 600 includes an elongated body portion 603 and a distal tip portion 605. The elongated body portion 603 extends from the distal tip portion 605 to the proximal end 604 end the delivery catheter 600. The distal tip portion 605 extends along a length of the delivery catheter 600 from the distal end 606 to the elongated body portion 603.

The outer diameter of the delivery catheter 600 is constant along the axial length of the elongated body portion 603 and the distal tip portion 605. In the example shown in FIG. 6, the outer diameter of the distal tip portion 605/body portion 603 is about 5.4 mm. But in other examples the outer diameter of the distal tip portion 605/body portion 603 ranges between about 4.5 mm and about 6.0 mm or between about 5.0 mm and about 6.0 mm. In another example, the outer diameter of the distal tip portion 605/body portion 603 is about 5.59 mm. In the example shown in FIG. 3, the inner diameter of the central lumen 602 of the delivery catheter 600 is about 4.4 mm But in other examples, the inner diameter of the central lumen 602 of the delivery catheter 600 ranges between 3.5 mm and about 5.0 mm or between 4.0 mm and about 4.5 mm.

In the example shown in FIG. 6, the length of the distal tip portion 605, which is measured in a direction along the longitudinal axis of the delivery catheter 600 is about 285.75 mm (11.25 inches). But in some examples, the distal tip portion 605 has a length less than 508 mm (20 inches), less than 381 mm (15 inches), between 127 mm (5 inches) and 381 mm (15 inches), or between 254 mm (10 inches) and 381 mm (15 inches). In the example shown in FIG. 6, the length of the body portion 603 is about 1041.4 mm (41 inches). But, in some examples, the length of the body portion 603 ranges between about 762 mm (30 inches) to about 1270 mm (50 inches) or between about 889 mm (35 inches) to about 1143 mm (45 inches).

As described above, the braided layer 626 provides structural support for the delivery catheter 600. The braided layer 626 also provides resistance to compressive deformation due to a varied density in the braid pattern. The braided layer 626 has a proximal end 630 and a distal end 632. The braided layer 626 includes a plurality of fibers 634 interwoven into a braided pattern and defines a central lumen 602, an inner surface 638, and an outer surface 640. In the example shown in FIG. 6, the braided layer 626 extends along the entire length of the delivery catheter 600. In other examples, the braided layer 626 extends along an entire length of the distal tip portion 605. The braided layer 626 extends along at least a portion of the entire length of the body portion 603 of the delivery catheter 600. In the example shown in FIG. 6 the braided layer 626 extends along the entire length of the delivery catheter 600.

As described above, the braided layer 626 is composed of a plurality of interwoven fibers 634. Each of the fibers 634 has a curvilinear cross section, although in other examples, the fibers 634 have a rectilinear cross section. The fibers 634 can also be flat wires to promote secure coupling of the fibers 634 in the braided pattern due to increased contact surface. The fibers 634 are composed of stainless steel, but in other examples, the fibers 634 are composed of polyester or at least one of a polymer, a metal, a composite, or any other material suitable for composing a stiffening braided layer 626 in a delivery catheter 600. Each of the plurality of interwoven fibers 634 has a diameter of about 0.004 inches. In some examples, each of the plurality of interwoven fibers 634 has a diameter ranging between about 0.0015 inches and about 0.006 inches, between about 0.003 inches and about 0.004 inches, or between about 0.002 inches and about 0.006 inches, or between about 0.003 inches and about 0.005 inches. The interwoven fibers 634 can be oriented in a single strand braid pattern or in a double strand braid pattern where each interweave includes a plurality of parallel fibers 634 for each weave strand. The plurality of interwoven fibers 634 is composed of 16 individual fibers 634. But in other examples the plurality of interwoven fibers 634 is composed of about 10 to about 40 individual fibers 634, or about 16 to about 32 individual fibers 634.

As described above, the braided layer 626 has a varied braid density which allows for omnidirectional bending with respect to the central axis of the delivery catheter 600. As the braid density increases, the braided layer 626 becomes more resistant to bending and axial compression. In the example shown in FIG. 6, the braid density of the braided layer 626 along the body portion 603 is less than a braid density of the braided layer 626 along the distal tip portion 605. This allows the distal tip portion 605 to conform to the vascular structure of a patient, while the body portion 603 is rigid enough to be manipulated by a physician. As described above, this increased braid density along the distal tip portion 605 provides the distal tip portion 605 with a greater resistance to axial compression and bending than the body portion 603. In the example shown in FIG. 6, the braid density along the entire length of the braided layer 626 varies between about 65 picks per inch and about 25 picks per inch. In other examples, the braid density along an entire length of the braided layer 626 varies between about 80 picks per inch and about 10 picks per inch. In some examples, the braid density along the distal tip portion 605 ranges between about 80 picks per inch and about 50 picks per inch or between about 60 picks per inch and about 65 picks per inch.

In some examples, the braid density along the body portion 603 ranges between about 35 picks per inch and about 10 picks per inch or between about 25 picks per inch and about 20 picks per inch. The plurality of interwoven fibers 634 are woven as individual fibers 634 into the braided pattern. But in other examples, the plurality of interwoven fibers 634 are woven as fibers 634 pairs into the braided pattern, such that two braid strands are used in place of a single braid strand in the example shown in FIG. 6. In the example shown in FIG. 6, the plurality of interwoven fibers 634 extend helically about the circumference of the braided layer 626. The plurality of fibers 634 have a pitch in the distal tip portion 605 that is less than a pitch of the plurality of fibers 634 in the body portion 603. A first portion of the plurality of interwoven fibers 634 extend helically about the circumference of the braided layer 626 in a first circumferential direction, and a second portion of the plurality of interwoven fibers 634 extend helically about the circumference in a second circumferential direction opposite the first circumferential direction to form the braided pattern. In some examples, the first portion of the plurality of interwoven fibers 634 extend helically about the circumference in the first direction at a first pitch, and the second portion of the plurality of interwoven fibers 634 extend helically about the circumference in the second direction at a second pitch where the first pitch is greater than the second pitch. This creates an axially narrower braid pattern that provides increased resistance to axial compression.

As described above, the delivery catheter 600 as shown in FIG. 6 includes at least one tensile stiffening fiber 627 having a proximal end 628 and a distal end 629. The tensile stiffening fiber 627 limits tensile deformation along the axial length of the delivery catheter 600 and limits bending of the delivery catheter 600 is limited at a location/along the side corresponding to the circumferential location of the tensile stiffening fiber 627. For example, if the tensile stiffening fiber is located at a 90-degree position (e.g., 3 o'clock) around the circumference of the delivery catheter 600, the delivery catheter 600 will resist bending at the circumferentially 90-degree position (e.g., 3 o'clock)

The tensile stiffening fiber 627 extends along at least a portion of the entire length of the body portion 603 of the delivery catheter 600. In the example shown in FIG. 6 the tensile stiffening fiber 627 extends along an entire length the body portion 603 of the delivery catheter 600. But, in other examples, a length of the at least one tensile stiffening fiber 627 corresponds to a length of the distal tip portion 605 or is greater than the length of the distal tip portion 605.

As described above with respect to delivery catheter 300, the tensile stiffening fiber 627 is similarly coupled to the delivery catheter 600 by the weaving/intertwining the tensile stiffening fiber 627 into the braid pattern of the braided layer 626. The tensile stiffening fiber 627 is further secured to the braided layer 626 when the outer jacket 608 is reflowed into the braided layer 626. Like delivery catheter 300, the tensile stiffening fiber 627 is fixedly coupled to the proximal and distal ends of the delivery catheter 600. In the example shown in FIG. 6 the at least one tensile stiffening fiber 627 is woven into the braided layer 626 along an entire length of the braided layer 626. But, in other examples, it is contemplated that the tensile stiffening fiber 627 is woven into a portion of the length of the braided layer 626. For example, the at least one tensile stiffening fiber 627 may not be woven into a first portion of the braided layer 626 extending along the distal tip portion 605 of the delivery catheter 600, but will be woven into a second portion of the braided layer 626 extending along an elongated body portion 603. The example shown in FIG. 6 includes Technora® Kevlar. But other examples include a tensile stiffening fiber 627 composed of other Kevlar, Liquid Crystal Polymer (LCP), a polyimide type polymer, or at least one of a polymer, a metal, a composite.

The example shown in FIG. 6 includes two tensile stiffening fibers 627 spaced around the circumference of the delivery catheter 600 by 180 degrees. But other examples include six or less, four or less, or one individual tensile stiffening fiber 627. In examples having a plurality of tensile stiffening fibers 627, the plurality of individual tensile stiffening fibers 627 can be equally spaced around the circumference of the delivery catheter 600 (e.g., every 60 degrees, 90 degrees, 120 degrees, 180 degrees), or irregularly spaced out. In examples having a one individual tensile stiffening fiber 627, the one individual tensile stiffening fiber 627 defines the neutral axis of the delivery catheter 600 (i.e., the axis or plane along the delivery catheter 600 at which no extension or compression occurs when the catheter is bent).

As described above, the delivery catheter 600 includes an outer jacket 608, which forms the outermost layer of the delivery catheter 600. The outer jacket 608 is composed of an elastic material Like delivery catheter 300, the elasticity of the outer jacket 608 varies (longitudinally) along a length of the outer jacket 608. In the example shown in FIG. 6, the elasticity of the outer jacket 608 increases between the proximal and distal end 611 of the outer jacket 608 such that the distal end 606 (e.g., along distal tip portion 605) of the delivery catheter 600 more easily bends to conform to the aortic geometry of a patient Like delivery catheter 300, the durometer of the outer jacket 608 decreases between the proximal end 604 and the distal end 606 of the delivery catheter 600. However, as described above, the distal most and proximal most segments of the delivery catheter 600 is constructed from a material having a higher durometer (and lower elasticity) than the next adjacent segment to facilitate coupling of the tensile stiffening fiber 627 to the proximal and distal ends of the delivery catheter 600.

The delivery catheter 600 includes a liner 633 provided on an inner surface of the outer jacket 608 similar to liner 333 described in the examples described above. The inner surface of the liner 633 defines an inner lumen of the delivery catheter 600. The liner 633 extends along an entire length of the inner lumen of the delivery catheter 600. In some examples, the liner 633 has a length that is greater than a length of the distal tip portion 605. In some examples, the liner 633 extends along at a portion of the entire length of the body portion 603 of the delivery catheter 600 or the entire length of the body portion 603 of the delivery catheter 600. The liner 633 in the example shown in FIG. 6, has a thickness of about 0.002 inches. But in other examples, the liner 633 has a thickness ranging between 0.001 inches and about 0.004 inches or between 0.0015 inches and about 0.003 inches. The liner 633 is composed of polytetrafluoroethylene (PTFE), Polyamide 12 materials, polyether block amide (PEBA), fluorinated ethylene propylene (FEP), or any other polymer suitable for forming an inner most layer of a flexible delivery catheter 600.

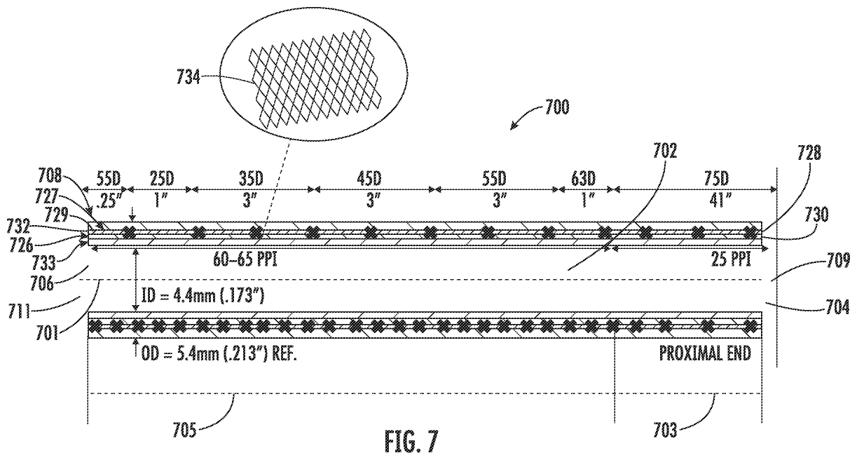

FIG. 7 shows another example of the flexible delivery catheter 700 including a braided layer and tensile stiffening fiber. The delivery catheter 700 includes similar structures and features to delivery catheters 300, 600 described above, the differences will be discussed in more detail below, like element numbers will be used to identify like elements. The delivery catheter 700 has an outer jacket 708, having a proximal end 704, a distal end 706, an inner surface 709 and an outer surface 710, and defining a central lumen 712 extending longitudinally between the proximal end 704 and the distal end 706. The delivery catheter 700 includes a braided layer 726 embedded in the outer jacket 708. The braided layer 726 includes a plurality of interwoven fibers 734 disposed in a braided pattern forming an elongated tubular structure defining a central lumen 702 extending therethrough. In some examples, the braided layer 726 includes constant pitch density along the entire length of the delivery catheter 700. The delivery catheter 700 includes at least one tensile stiffening fiber 727 extending longitudinally along the delivery catheter 700 from the proximal end 704 to the distal end 706 of the delivery catheter 700. The at least one tensile stiffening fiber 727 limits bending of the delivery catheter 700 in a direction opposite the circumferential location of the at least one tensile stiffening fiber 727. For example, if the tensile stiffening fiber is located at a 90-degree position (e.g., 3 o'clock) around the circumference of the delivery catheter 700, the delivery catheter 700 will resist bending at the circumferentially 90-degree position (e.g., 3 o'clock). The tensile stiffening fiber 727 extends along at least a portion of the entire length of the body portion 703 of the delivery catheter 700. In the example shown in FIG. 7 the least one tensile stiffening fiber 727 extends along an entire length the body portion 703 of the delivery catheter 700. But, in other examples, a length of the at least one tensile stiffening fiber 727 corresponds to a length of the distal tip portion 705 or is greater than the length of the distal tip portion 705. The tensile stiffening fiber 727 is coupled to the delivery catheter 700 by the weaving/intertwining the tensile stiffening fiber 727 into the braid patter of the braided layer 726. The distal ends of the tensile stiffening fiber 727 is fixedly coupled to the outer jacket 708 when the outer jacket 708 is reflowed into the braided layer 726 during manufacturing.

As shown in FIG. 7, and similar to delivery catheters 300 and 600, the elasticity of the outer jacket 708 increases between the proximal end and distal end 711 of the outer jacket 708 such that the distal end 706 of the delivery catheter 700 is configured to conform to the aortic geometry of a patient. The durometer of the outer jacket 708 decreases between the proximal end 704 and the distal end 706 of the delivery catheter 700. As such, the outer jacket 708 includes various longitudinal segments which are constructed from materials having varying durometer. The materials of the various longitudinal segments are reflowed together to form a single outer jacket 708. The distal end 711 of the outer jacket 708 includes a coupling segment which has lower elasticity and higher durometer than an adjacent segment of the outer jacket 708. This segment has such a durometer to provide a material capable of suitable to securely fasten the tensile fiber 727 to. In the example shown in FIG. 7 the outer jacket 708 is composed of pebax. But, in other examples, at least a portion of the outer jacket 708 comprises another polyether block amide, another elastomer, a polyimide (e.g., Vestamid®), Rilsamid PA12, or any other material suitable for forming the outermost layer of a flexible delivery catheter 700. The delivery catheter 700 includes a liner 733. The liner 733 extends along an entire length of the inner lumen of the delivery catheter 700 and along the inner surface of the outer jacket 708. In other examples which include a braided layer 726, the delivery catheter 700 includes a liner 733 on an inner surface of the braided layer 726.

FIGS. 14A and 14B show another example of the flexible delivery catheter 900. The delivery catheter 900 includes similar structures and features to delivery catheter 300 described above, the differences will be discussed in more detail below, like element numbers will be used to identify like elements. As with the example delivery catheters described herein, the flexible delivery catheter 900 includes an outer jacket 908 having various elasticity characteristics along its axial length, a coil layer 918 which provides compressive stiffness to the catheter, a braided layer 926 which provides structural rigidity, a tensile stiffening fiber 927 which provides tensile stiffness. The delivery catheter 900 includes a delivery capsule 950 which houses a medical device, and a coupler 960 which joins the delivery capsule 950 and the delivery catheter 900 keeping the coil layer 918 disposed within the outer jacket 908. The delivery catheter 900 has a proximal end 904 and a distal end 906, an inner surface 907, and an outer surface 910. The delivery catheter 900 defines an elongated tubular structure with a central lumen 902 extending longitudinally between the proximal end 904 and the distal end 906. The central lumen 902 is configured for passage of medical devices such as prosthetic heart valves therethrough. The delivery catheter 900 includes an elongated body portion 903 and a distal tip portion 905. The elongated body portion 903 extends from the distal tip portion 905 (at the distal end 906 of the delivery catheter 900) to the proximal end 904 of the delivery catheter 900. The elongated body portion 903 provides a section of the delivery catheter 900 that is rigid enough for effective manipulation by a physician (e.g., advancement through the patient's vasculature). The delivery catheter 900 is omnidirectionally flexible and configured to bend in any direction away from a longitudinal axis 901 of the delivery catheter 900 while retaining compressive and tensile stiffness. As such, the flexible delivery catheter 900 conforms to a patient's vascular structure and during placement and delivery of a medical device (e.g., prosthetic heart valve), while limiting axial deformation of the delivery catheter 900 that can damage a patient's vascular structure.

The delivery catheter 900 includes an elongated body portion 903 and a distal tip portion 905. The elongated body portion 903 extends from the distal tip portion 905 to the proximal end 904 end of the delivery catheter 900. The distal tip portion 905 extends along a length of the delivery catheter 900 from the distal end 906 of the delivery catheter 900 to the elongated body portion 903. In the example shown in FIGS. 14A and 14B, as discussed further below, the distal tip portion 905 corresponds to the axial location of the cavity/second portion of the outer jacket 908, and the elongated body portion 903 corresponds to the first portion of the outer jacket 908. Also, in some examples, and as illustrated in FIG. 14A, the outer diameter of the distal tip portion 905 corresponds with the outer diameter of the elongated body portion 903, such that a smooth transition is formed along the outer surface of the delivery catheter 900 between the elongated body portion 903 and distal tip portion 905. In other examples, the outer diameter of the distal tip portion 905 is greater than the outer diameter of the elongated body portion 903, such that the outer diameter of the delivery catheter 300 increases from the elongated body portion 903 to the distal tip portion 905 to the delivery capsule 950. In this example, the outer diameter of the elongated body portion 903 may or may not taper radially outwardly to the outer diameter of the distal tip portion 905. In further alternative examples, the outer diameter of the distal tip portion 905 is less than the outer diameter of the elongated body portion 903.

In the example shown in FIGS. 14A and 14B, the outer diameter of the distal tip portion 905 and elongated body portion 903 is about 5.4 mm But in other examples, the outer diameter of the body portion 903 ranges between about 4.5 mm and about 6.0 mm or between about 5.0 mm and about 6.0 mm. In other examples, the outer diameter of the distal tip portion 905 ranges between about 4.5 mm and about 6.0 mm, or between about 5.0 mm and about 6.0 mm.

As described above, the delivery catheter 900 includes an outer jacket 908, which forms the outermost layer of the delivery catheter 900. The outer jacket 908 has a proximal end 909, a distal end 911, an inner surface 912, and an outer surface 913. The outer jacket 908 defines a central lumen 914 extending longitudinally between the proximal end 909 and the distal end 911 and is composed of an elastic material. The outer jacket 908 further includes a first outer jacket portion 915 and a second outer jacket portion 916. The first outer jacket portion 915 is adjacent the proximal end 909 of the outer jacket 908, extends along at least a portion of the entire length of the body portion 903, and has a first inner diameter within the central lumen 914 of the outer jacket 908. The second outer jacket portion 916 is adjacent the distal end 911 of the outer jacket 908, extends along at least a portion of the distal tip portion 905. The second outer jacket portion 916 has a second inner diameter within the central lumen 914 of the outer jacket 908. In the example illustrated in FIG. 14A, the first inner diameter is less than the second inner diameter such that the change in inner diameter between the first inner diameter and the second inner diameter creates a shoulder 917 between the first outer jacket portion 915 and the second outer jacket portion 916.

In the example shown in FIGS. 14A and 14B, the first inner diameter of the first outer jacket portion 915 is about 4.4 mm But in other examples, the first inner diameter of the first outer jacket portion 915 ranges between about 3.5 mm and about 5 mm or between 4.0 mm and about 4.5 mm. The second inner diameter of the second outer jacket portion 916 shown in FIGS. 14A and 14B is 4.8 mm, but in other examples, the second inner diameter of the second outer jacket 916 ranges between about 4.6 mm to about 5.4 mm. Also, in the example shown in FIGS. 14A and 14B, the axial location of the second outer jacket portion 916 corresponds, at least in part, to the location of the distal tip portion 905 of the delivery catheter 900.

The length of the second outer jacket portion 916 is less than the length of the first outer jacket portion 915 of the delivery catheter 900. In the example shown in FIGS. 14A and 14B, the overall length of the outer jacket 908 is 1320.8 mm (52 inches), but in other examples, the overall length of the outer jacket 908 ranges between about 762 mm (30 inches) to about 1178 mm (70 inches) or between 889 mm (35 inches) to 1397 mm (55 inches).

Like delivery catheter 300 and other catheters described herein, the elasticity of the outer jacket 908 varies (longitudinally) along a length of the outer jacket 908. In the example of FIGS. 14A and 14B, the elasticity of the outer jacket 908 varies between the proximal end 909 and distal end 911 of the outer jacket 908 such that the distal end 906 (e.g., along distal tip portion 905) of the delivery catheter 900 more easily bends to conform to the aortic geometry of a patient. Like delivery catheter 300, the durometer of the outer jacket 908 decreases between the proximal end 904 and the distal end 906 of the delivery catheter 900. The outer jacket 908 includes various longitudinal segments (e.g., segments 908a-908e) which are constructed from materials having varying durometer. The materials of the various longitudinal segments 908a-908e (with varying durometers) are reflowed together to form a single outer jacket 908.

In the example shown in FIGS. 14A and 14B, the outer jacket 908 is composed of Pebax. But, in other examples, at least a portion of the outer jacket 908 comprises another polyether block amide, another elastomer, a polyamide (e.g., Vestamid®), or any other material suitable for forming the outermost layer of a flexible catheter.

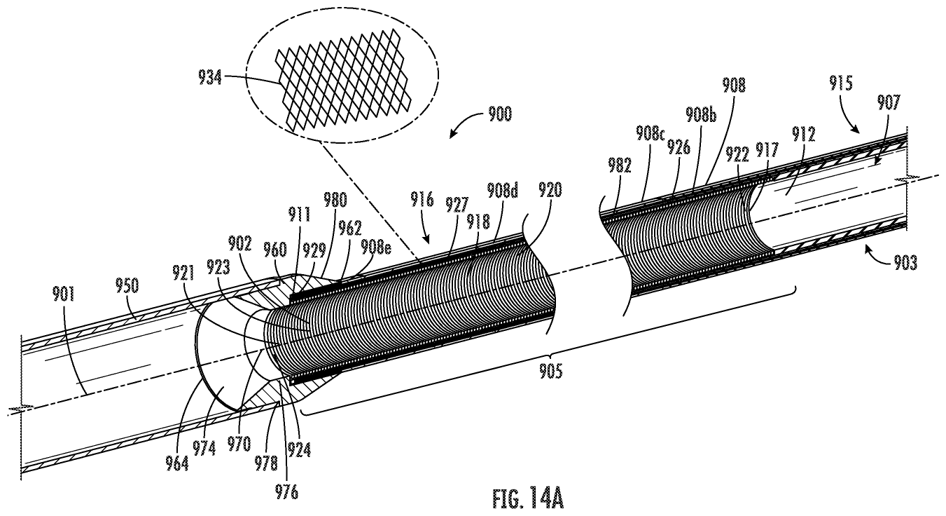

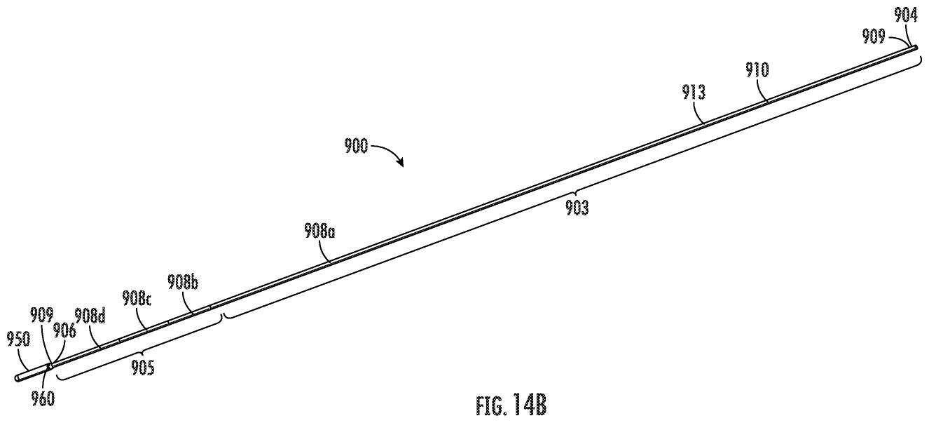

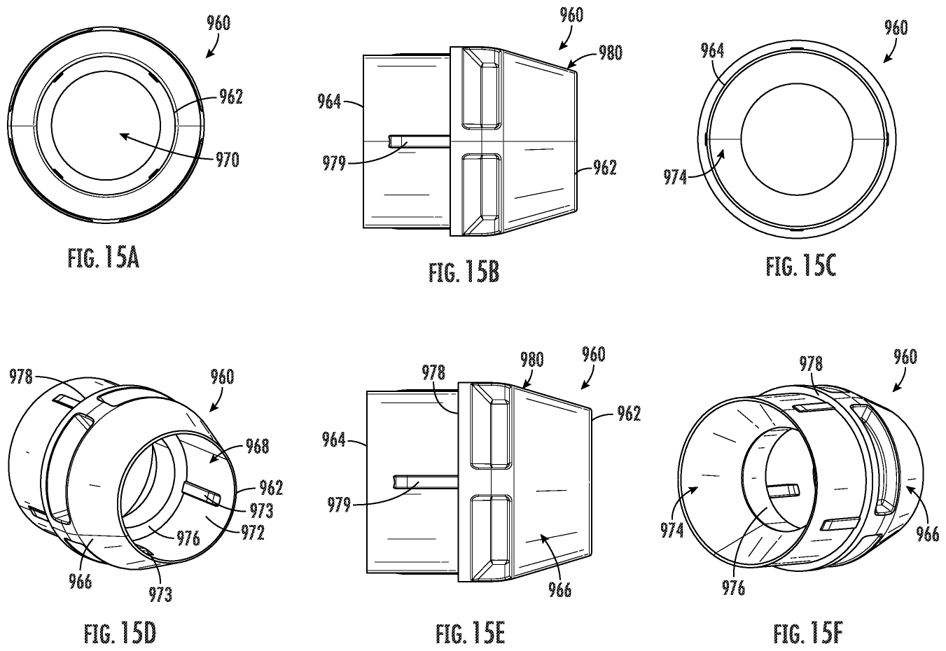

The coupler 960, as shown in FIGS. 14A-15F, joins the delivery catheter 900 to the delivery capsule 950. The coupler 960 has a body with a proximal end 962 and a distal end 964, an outer surface 966, and an inner surface 968. The inner surface 968 of the coupler 960 defines a central lumen 970 that extends longitudinally between the proximal end 962 and the distal end 964 of the coupler 960. The central lumen 970 of the coupler includes a cylindrical portion 972 adjacent the proximal end 962 of the coupler 960, an increasing taper portion 974 adjacent the distal end 964 of the coupler 960. A radially extending protrusion/shoulder 976 is provided between the cylindrical portion 972 and the increasing taper portion 974. As illustrated in FIG. 15D, the diameter of the radially extending protrusion/shoulder 976 is less than the diameter of the cylindrical portion 972. The central lumen of the coupler 960 also includes at least one rib 973 projecting inward from the inner surface of the central lumen 970. The ribs 973 helps improve purchase/engagement between the coupler 960 and the delivery catheter 900. In one example, the ribs 973 form a generally rectangular shape extending along a portion of the cylindrical portion 972. The rib 973 includes a tapered proximal edge to help ease introduction of the delivery catheter 900 into the central lumen of the coupler 960.

The outer surface 966 of the coupler 960 includes a recess 978 that extends around the entire circumference of the outer surface 966 of the coupler 960 and a decreasing taper 980 that starts between the recess 978 and proximal end 962 of the coupler 960 and extends axially and radially inwardly towards the proximal end 962 of the coupler 960. The distal end of the taper 980 has a generally cylindrical shape with a diameter greater than the diameter of the recess 978 (e.g., cylindrical portion). The recess 978 on the outer surface 966 of the coupler 960 is configured to receive the delivery capsule 950. The recess 978 includes a reduced diameter cylindrical portion that abuts a shoulder formed between the recess 978 and the tapered portion. In one example, at least one rib 979 projecting outward from the outer surface of the cylindrical portion of the recess 978. The ribs 979 help improve purchase/engagement between the coupler 960 and the delivery capsule 950. As illustrated in FIGS. 15A-15G, the ribs 979 form a generally rectangular shape that extends along a portion of the recess 978. The ribs 979 include a tapered distal edge to help ease introduction of the delivery capsule onto the recess 978 of the coupler 960.

When assembled, the proximal end 962 of the coupler 960 is disposed adjacent the distal end 911 of the outer jacket 908 such that the radially extending protrusion/shoulder 976 on the inner surface 968 of the coupler 960 abuts the distal end 911 of the outer jacket 908. The radially extending protrusion/shoulder 976 on the inner surface 968 of the coupler 960 and the second portion 916 of the outer jacket 908 form a cavity 982 that the coil layer 918 is disposed within. In some examples, the inner diameter of the shoulder 917 formed in central lumen the outer jacket 908 corresponds to the inner diameter of the central lumen of the coil layer 918. It is further contemplated that the radially extending protrusion/shoulder 917 of the outer jacket 908 can include a taper.

When the delivery catheter 900 is assembled, the distal end 964 of the coupler 960 is disposed adjacent a delivery capsule 950 such that the recess 978 on the outer surface of the coupler 960 receives the delivery capsule 950. The central lumen of the delivery capsule 950 is sized and configured to receive a prosthetic heart valve in a crimped or non-expanded configuration. Example delivery capsules are described, for example, in U.S. Pat. No. 9,867,700, entitled “Prosthetic Heart Valve Delivery Apparatus,” U.S. Pat. No. 10,695,176, entitled “Prosthetic Heart Valve Delivery Apparatus,” U.S. Pat. No. 10,376,364, entitled “Implant Delivery Capsule” the contents of which are incorporated herein by reference in their whole entirety. In the example shown in FIGS. 14A and 14B, the outer diameter of the delivery capsule is 7.9 mm, but in other examples, the outer diameter of the delivery capsule ranges from about 7 mm to about 9 mm. The outer diameter of the delivery capsule 950 corresponds to the outer diameter of the coupler 960 and is greater than the outer diameter of the outer jacket 908.

It is contemplated that the coupler 960 can be coupled to the delivery capsule 950 by at least one of a press fit, an interference fit, a snap fit, a mechanical fastener, a weld, and an adhesive and that the coupler 960 can be coupled to the outer jacket 908 by at least one of a press fit, an interference fit, a snap fit, a mechanical fastener, a weld, and an adhesive.

The coil layer 918 is wound to resist axial compression and tension applied to the delivery catheter 900, while facilitating bending of the delivery catheter 900 in a direction away from the longitudinal axis of the delivery catheter 900. The coil layer 918 includes a coil winding 920 which provides compressive stiffness to the delivery catheter 900 during axial compressive loads, a proximal end 922, and a distal end 924. The coil winding 920 extends helically about a longitudinal axis 921 and defines a central lumen 923 between the proximal end 922 and the distal end 924. In the example shown in FIGS. 14A and 14B, the coil layer 918 has a plurality of tightly wound turns of the coil winding 920. In an example coil layer 918, a gap/spacing is provided between adjacent turns of the coil winding 920, and the maximum gap/spacing between the adjacent turns of the coil winding is 0.25 mm (0.010 inches). The coil layer 918 is tightly wound such that the adjacent turns of the coil winding 920 contact, i.e., an outer surface of the adjacent turns of the coiled winding 920 contact along a circumferential length of the coiled winding 920. This allows for some axial compression in certain applications that require partial axial compression of the delivery catheter 900.

The coil layer 918 is disposed within the cavity 982 formed between the outer jacket 908 and coupler 960 such that the outer surface of the coil layer 918 abuts the inner surface 912 of the second outer jacket portion 916, the proximal end 922 of the coil layer 918 abuts the shoulder 917 between the first outer jacket portion 915 and the second outer jacket portion 916, and the distal end 924 of the coil layer 918 abuts the radially extending protrusion/shoulder 976 on the inner surface 968 of the coupler 960. In some examples, the inner diameter of the coil layer 918 is equal to the first inner diameter of the outer jacket 908 at the location corresponding to the cavity/first outer jacket portion 915, and the outer diameter of the coil layer 918 is less than the second inner diameter of the outer jacket 908 at the location corresponding to the second outer jacket portion 916. In further examples, the outer diameter of the coil layer 918 corresponds to the second inner diameter of the outer jacket 908 and is greater than the first inner diameter of the outer jacket 908. It is also contemplated that the inner diameter of the coil layer 918 is greater than the first inner diameter of the outer jacket 908. The outer diameter of the coil layer 918 ranges between about 4.6 mm to about 5.4 mm.

In the example shown in FIGS. 14A and 14B, the coil winding 920 has a diameter ranging between about 0.003 inches and about 0.010 inches, and between about 0.004 inches and about 0.008 inches. In one example the coil winding 920 has a diameter of about 0.004 inches. In other examples, the coil winding 920 has a diameter of about 0.008 inches.

As described above, the coil windings 920 have a constant pitch along the axial length of the coil layer 918. But in other implementations, the coil windings 920 have a varying pitch along an entire length of the coil winding 920/coil layer 918. For example, a pitch of the coil winding 920 at a proximal end of the distal tip portion 905 can be less than a pitch of the coil winding 920 at the distal end 906 of the distal tip portion 905. In the example shown in FIGS. 14A and 14B the coil layer 918 has about 55 turns per inch, but in other examples, the coil has between about 50 and 120 turns per inch, in implementations having 0.008 inch diameter coil wire.

In the example shown in FIGS. 14A and 14B, the coil layer 918 has a length that is equal to the length of the distal tip portion 905 and equal to the length of the cavity 982. In some examples, the length of the coil layer 918 corresponds to the length of the distal tip portion 903 and/or the length of the cavity 982. But in other examples, a length of the coil layer 918 is less than the length of the distal tip portion 905 and/or cavity 982. In further examples, the length of the coil layer 918 is greater than a length of the distal tip portion 905 and/or cavity such that the coil layer 918 extends into the body portion 903 of the delivery catheter 900.

In the example shown in FIGS. 14A and 14B, the coil layer, which is measured in a direction along the longitudinal axis of the delivery catheter 900 is about 215.9 mm (8.5 inches). In some examples, the coil layer has a length less than 305 mm (12 inches), less than 254 mm (10 inches), between 76 mm (3 inches) and 254 mm (10 inches), or between 178 mm (7 inches) and 254 mm (10 inches).

The coil layer 918 can be composed of at least one of a polymer, a metal such as 300 or 400 series stainless steel, or a composite. For example, the coil layer 918 can be composed of a stainless steel wire. The coil winding 920 has a curvilinear shape in cross section, but the coil winding 920 can have either a rectilinear or curvilinear shape in cross section.

As described above the delivery catheter 900 includes a braided layer 926 which provides structural support for the delivery catheter 900 such that the distal tip portion 903 will not deform when receiving axial load from the coil layer 918. The braided layer 926 has a proximal end 930 and a distal end 932. The braided layer 926 includes a plurality of fibers 934 interwoven into a braided pattern and defines a central lumen 936, an inner surface 938, and an outer surface 940. In the example shown in FIGS. 14A and 14B, the braided layer 926 extends along the entire length of the outer jacket 908 of the delivery catheter 900. In another example, the braided layer 926 extends along an entire length of the distal tip portion 905 and then extends down into the first outer jacket portion 915 towards the central lumen 914 of the outer jacket 908. In yet another example, the braided layer 926 extends along at least a portion of the distal tip portion 905 and/or at least a portion of the elongated body portion 903 such that the braided layer 926 extends over a corresponding length of the coil layer 918 but not the entire length of the outer jacket 908. In the example shown in FIGS. 14A and 14B, the braided layer 926 extends along the entire length of the delivery catheter 900. As such, braided layer 926 has a length that is greater than an overall length of the coil layer 918. But, in some implementations the braided layer 926 and the coil layer 918 are about the same length along the axis of the delivery catheter 900.

As described above, the braided layer 926 is composed of a plurality of interwoven fibers 934. Each of the fibers 934 has a curvilinear cross section, although in other examples, the fibers 934 have a rectilinear cross section. The fibers 934 are composed of a liquid crystal polymer, but in other examples, the fibers 934 are composed of at least one of a polyester, a polymer, a metal (e.g. stainless steel), a composite, Kevlar (e.g., Technora®), or any other material suitable for composing a stiffening braided layer 926 in a delivery catheter 900. Each of the plurality of interwoven fibers 934 has a diameter of about 0.004 inches. In other examples, each of the plurality of interwoven fibers 934 has a diameter ranging between about 0.002 inches and about 0.006 inches or between about 0.003 inches and about 0.005 inches. The interwoven fibers 934 can be oriented in a single strand braid pattern or in a double strand braid pattern where each interweave includes a plurality of parallel fibers 934 for each weave strand. The plurality of interwoven fibers 934 is composed of 16 individual fibers 934. But in other examples the plurality of interwoven fibers 934 is composed of about 10 to about 20 individual fibers 934. The braided layer 926 has a braid density which allows for omnidirectional bending with respect to the central axis of the delivery catheter 900. As the braid density increases, the braided layer 926 becomes more resistant to bending. In the example shown in FIGS. 14A and 14B, the braid density of the braided layer 926 is about 25 picks per inch. But in other examples, the braided layer 926 has a braid density between about 20 to about 30 picks per inch.

As described above, the delivery catheter 900 as shown in FIGS. 14A and 14B includes at least one tensile stiffening fiber 927 having a proximal end 928 and a distal end 929. The tensile stiffening fiber 927 limits tensile deformation along the axial length of the delivery catheter 900 such that the bending of the delivery catheter 900 is limited at a location/along the side corresponding to the circumferential location of the tensile stiffening fiber 927. For example, if the tensile stiffening fiber 927 is located at a 90-degree position (e.g., 3 o'clock) around the circumference of the delivery catheter 900, the delivery catheter 900 will resist bending at the circumferentially 90-degree position (e.g., 3 o'clock). Generally, the location of the tensile stiffening fiber 927 determines the neutral bending axis of the delivery catheter 900. That is, the stiffness and relative location of the tensile stiffening fiber 927 (or location of multiple fibers with respect to each other) defines the line or plane along the delivery catheter 900 at which no extension or compression occurs when the catheter is bent. For example, when the delivery catheter 900 includes a single tensile stiffening fiber 927, the neutral axis of the delivery catheter 900 is defined by the axis of the tensile stiffening fiber 927.

The tensile stiffening fiber 927 extends along at least a portion of the entire length of the body portion 903 of the delivery catheter 900. In the example shown in FIGS. 14A and 14B, the at least one tensile stiffening fiber 927 extends along an entire length the body portion 903 of the delivery catheter 900. But, in other examples, a length of the at least one tensile stiffening fiber 927 corresponds to a length of the distal tip portion 905 or is greater than the length of the distal tip portion 905.

The tensile stiffening fiber 927 is coupled to the delivery catheter 900 by the weaving/intertwining the tensile stiffening fiber 927 within the fibers/braid pattern of the braided layer 926. The tensile stiffening fiber 927 is further secured to the braided layer 926 when the outer jacket 908 is reflowed into the braided layer 926 during manufacturing. As previously discussed, the distal end 906 of the delivery catheter 900 includes materials of varying durometer along the length of the outer jacket 908. A length/segment of the delivery catheter 900 proximate the distal end 906 of the distal tip portion 905 comprises a material having a higher durometer than the next adjacent segment of the delivery catheter 900. Reflow of the tensile stiffening fiber 927 with the higher durometer material at the distal end 906 of the distal tip portion 905 results in higher friction between the tensile stiffening fiber 927 and the higher durometer material of the outer jacket 908, thereby preventing axial slippage/movement of the tensile stiffening fiber 927 at the distal end 906 of the delivery catheter 900. Similarly, the proximal end 904 of the delivery catheter 900 includes a length/segment of material having a higher durometer than the next adjacent segment of the delivery catheter 900. Accordingly, reflow of the tensile stiffening fiber 927 with the higher durometer material at the proximal end 904 of the delivery catheter 900 prevents slippage/movement of the tensile stiffening fiber 927 with respect to the end of the delivery catheter 900. As such, the tensile stiffening fiber 927 is secured both along the length of the fiber and at its proximal and distal ends, i.e., the tensile stiffening fiber 927 is fixedly coupled to the proximal and distal ends of the outer jacket 908/braided layer 926 and along the along the braided layer 926.

As shown in FIGS. 14A and 14B, the tensile stiffening fiber 927 is woven into the braided layer 926 along an entire length of the braided layer 926. But, in other examples, it is contemplated that the tensile stiffening fiber 927 is woven into a portion of the length of the braided layer 926. For example, the at least one tensile stiffening fiber 927 may not be woven into a first portion of the braided layer 926 extending along the distal tip portion 905 of the delivery catheter 900 corresponding to the length of the coil layer but will be woven into a second portion of the braided layer 926 extending along the elongated body portion 903 of the delivery catheter 900 between the distal tip portion 905 and the proximal end 904 of the delivery catheter 900.

The example shown in FIGS. 14A and 14B includes Technora® Kevlar. But other examples, a tensile stiffening fiber 927 is composed of at least one of a polymer, a metal, a composite including a polyimide type polymer, Kevlar, or Liquid Crystal Polymer (LCP).

The example shown in FIGS. 14A and 14B includes four tensile stiffening fibers 927 spaced around the circumference of the delivery catheter 900 by 90 degrees. But other examples include six or less, four or less, two or less, or one individual tensile stiffening fiber 927. In examples having a plurality of tensile stiffening fibers 927, the plurality of individual tensile stiffening fibers 927 can be equally spaced around the circumference of the delivery catheter 900 (e.g., every 60 degrees, 90 degrees, 120 degrees, 180 degrees), or irregularly spaced around the circumference of the delivery catheter 900. In examples having a one individual tensile stiffening fiber 927, the one individual tensile stiffening fiber 927 is located at a neutral axis of the delivery catheter 900.

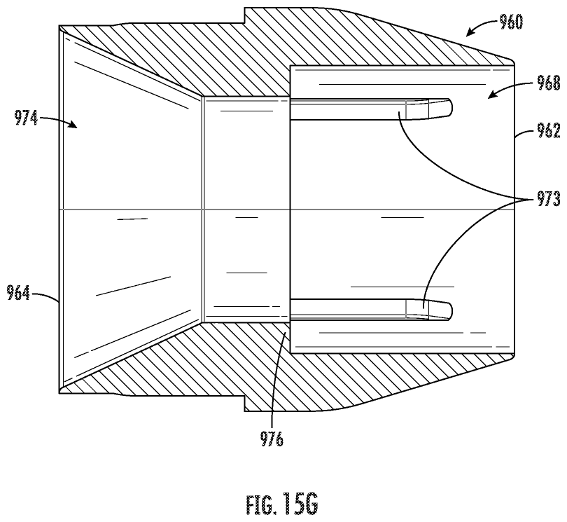

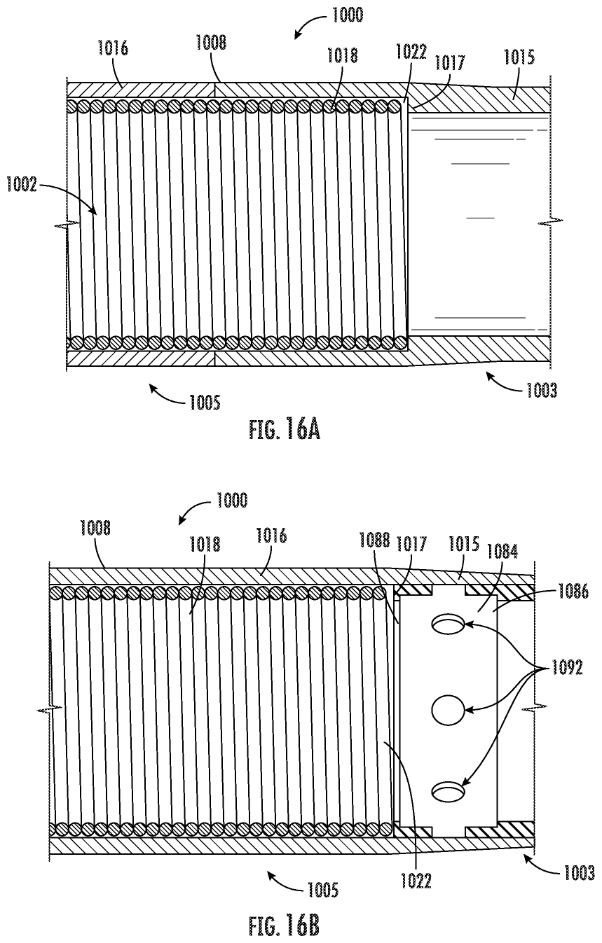

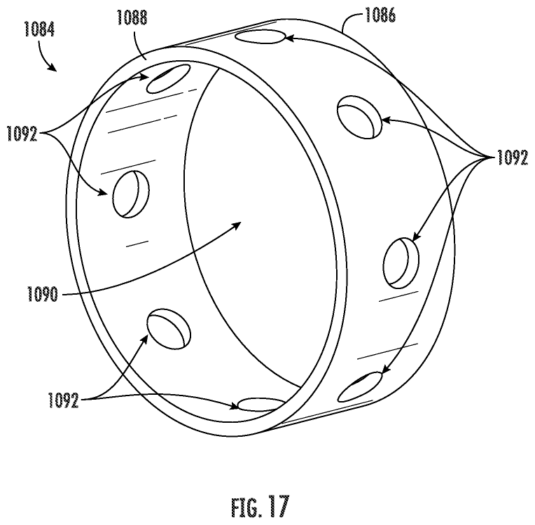

FIGS. 16A and 16B show another implementation of a delivery catheter 1000 that similar to the delivery catheter 900 but further includes a ring 1084 provided at the proximal end 1004 of the coil layer 1018, like element numbers will be used to identify like elements. The ring 1084 provides extra support for the coil layer 1018 and distal tip portion 1005 of the delivery catheter 1000. The ring 1084 as shown in FIG. 17 includes a proximal end 1086 and distal end 1088 with a central lumen 1090 extending longitudinally therethrough. The ring 1084 is embedded in and reflowed with the first outer jacket portion 1015 such that the distal end 1088 of the ring 1084 is adjacent the shoulder 1017 between the first outer jacket portion 1015 and second outer jacket portion 1016 and, thus, the proximal end 1022 of the coil layer 1018. The ring 1084 further includes eight openings 1092 equally spaced around the ring 1084 such that reflowed material can extend through the eight openings 1092 to facilitate the ring 1084 being reflowed and embedded within the outer jacket 1008. In other examples, the ring 1084 includes more or less than eight openings 1092. In further examples, the ring 1084 does not include any openings.

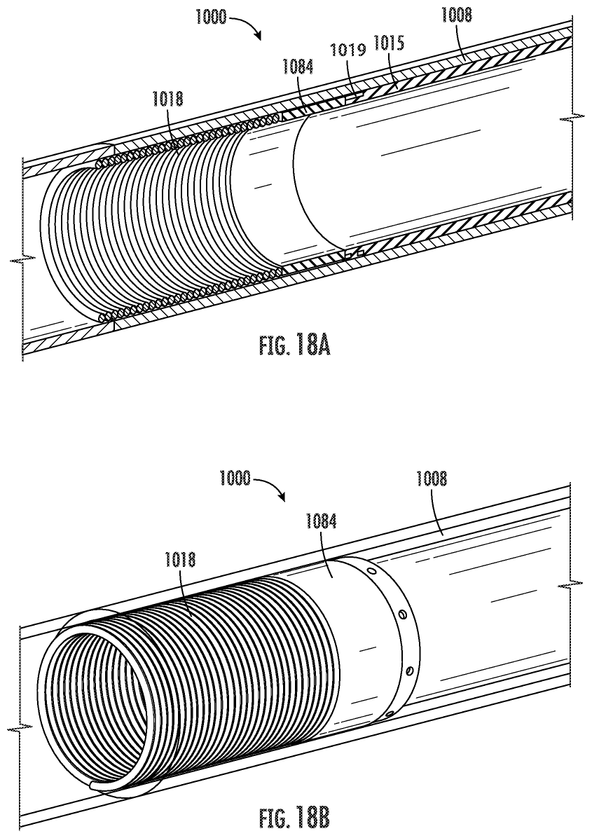

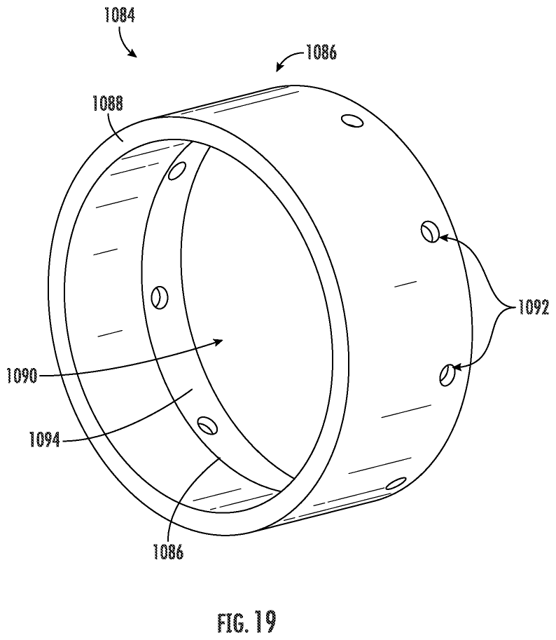

In other examples, the ring 1084 is only partially embedded in the first outer jacket portion 1015 and/or entirely disposed within the cavity of the outer jacket 1008 adjacent the proximal end of the coil layer 1018. For example, as illustrated in FIGS. 18A-18B and 19 showing a cross-section and transparent view of the delivery catheter 1000, respectively, ring 1084 can further include a circumferential extrusion 1094 extending from the proximal end 1086 of the ring 1084. The circumferential extrusion 1094 has a larger inner diameter than inner diameter of the ring 1084. As illustrated in FIG. 18A, the inner diameter of the ring 1084 corresponds to the inner diameter of the outer jacket 1008 such that there is a smooth transition between the ring 1084 and the outer jacket 1008.

As illustrated in FIG. 19, the openings 1092 of the ring 1084 are included on the circumferential extrusion 1094 such that the circumferential extrusion 1094 reflowed with the first outer jacket portion 1015 and only a portion of the ring 1084 is embedded within the outer jacket 1008. In some examples the first outer jacket portion 1015 includes a slot 1019 in the shoulder 1017 for receiving the ring 1084 and/or the circumferential extrusion 1094 of the ring 1084.

The ring shown in FIGS. 16A-19 is made of stainless steel, but in other implementations, the ring is composed of at least one of a polymer, metal, or composite.

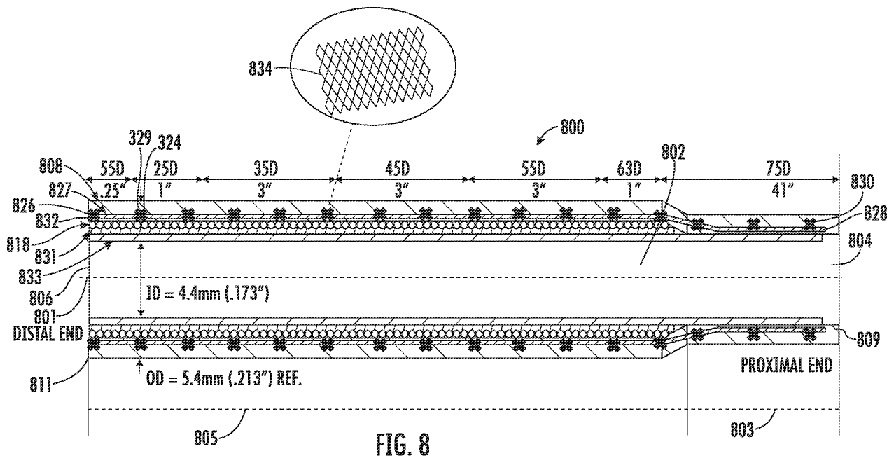

Referring to FIG. 8, FIG. 8 shows a flexible delivery catheter 800 including a hypotube 818 rather than a coiled layer. The delivery catheter 800 includes similar structures and features to delivery catheters 300, 600, 700 described above, the differences will be discussed in more detail below, like element numbers will be used to identify like elements. The delivery catheter 800 includes, an outer jacket 808 having a proximal end 809 and a distal end 811 and a hypotube 818. The hypotube 818 has a proximal end 819, a distal end 820, an inner surface 821, an outer surface 822. The hypotube 818 further includes plurality of cut-outs 823 which extend between the inner surface 821 and the outer surface 822 of the hypotube 818 such that the hypotube 818 can bend omnidirectionally. The hypotube 818 is provided at the distal tip portion 805 of the delivery catheter 800.

The delivery catheter 800, in particular the distal tip portion 805, is omnidirectionally flexible and configured to bend in any direction away from a longitudinal axis 801 of the delivery catheter 800 while retaining compressive and tensile stiffness. As such, the flexible delivery catheter 800 more easily bends to conform to the patient's vascular structure during an operation while also limiting axial deformation that can damage a patient's vascular structure. The delivery catheter 800 defines a central lumen 802 extending longitudinally between the proximal end 804 and the distal end 806. Like delivery catheters 300, 600, 700, the delivery catheter 800 includes an elongated body portion 803 and a distal tip portion 805. The distal tip portion 805 extends along a length (e.g., approx. 11.25 inches) of the delivery catheter 800 from a distal end 806 of the delivery catheter 800 toward the proximal end 804 of the delivery catheter 800, up to the elongated body portion 803 of the hypotube 818. The elongated body portion 803 extends from the distal tip portion 805 to the proximal end 804 end the delivery catheter 800 (e.g., approx. 41.0 inches). As shown in FIG. 8, the outer diameter of the distal tip portion 805 is greater than the outer diameter of the elongated body portion 803. The outer diameter/surface of the distal tip portion 805 adjacent the body portion 803 includes a decreasing taper between the larger outer diameter distal tip portion 805 and the reduced diameter body portion 803.

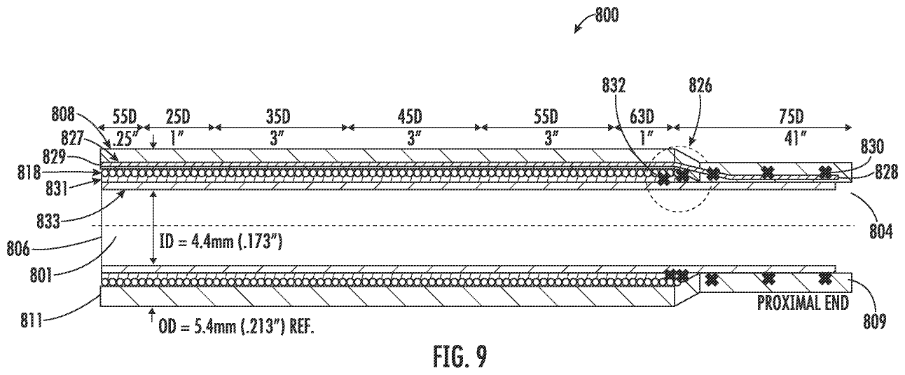

The hypotube 818 is disposed within the distal tip portion 805. As illustrated in FIG. 8, the hypotube 818 is located on the inner lumen of the braided layer 826, where the and the braided layer 826 is disposed between the outer jacket 808 and the hypotube 818. In some examples, as illustrated in FIG. 9, the hypotube 818 is located at the distal tip portion 805 and the braided layer 826 is located along the body portion 803. The braided layer 826 overlaps a with a portion of the proximal end of the hypotube 818. For example, the braided layer overlaps a length of the hypotube 818 by an axial length ranging between about 0.050 inches and about 0.100″ inches. As illustrated in FIG. 9, the overlapping portion of the braided layer 826 is provided on the inner diameter of the hypotube 818. It is also contemplated that the overlapping portion of the braided layer 826 can be provided on the outer diameter of the hypotube 818.

In the example shown in FIG. 8, the outer diameter of the body portion 803 is about 5.4 mm But in other examples the outer diameter of the body portion 803 ranges between about 4.5 mm and about 6.0 mm or between about 5.0 mm and about 6.0 mm. In the example shown in FIG. 8, the outer diameter of the distal tip portion 805 is about 5.59 mm But, in other examples, the outer diameter of the distal tip portion 805 ranges between about 4.5 mm and about 6.0 mm, or between about 5.0 mm and about 6.0 mm.

Like the example delivery catheter 300 shown in FIG. 3, the inner diameter of the central lumen of the delivery catheter 800 is about 4.4 mm But in other examples, the inner diameter of the central lumen of the delivery catheter 800 ranges between 3.5 mm and about 5.0 mm or between 4.0 mm and about 4.5 mm Similar to the catheter examples described herein, the length of the distal tip portion 805, measured in a direction along the longitudinal axis of the delivery catheter 800, is about 285.75 mm (11.25 inches). But in some examples, the distal tip portion 805 has a length less than 508 mm (20 inches), less than 381 mm (15 inches), between 127 mm (5 inches) and 381 mm (15 inches), or between 254 mm (10 inches) and 381 mm (15 inches). In the example shown in FIG. 8, the length of the body portion 803 is about 1041.4 mm (41 inches). But, in some examples, the length of the body portion 803 ranges between about 762 mm (30 inches) to about 1270 mm (50 inches) or between about 889 mm (35 inches) to about 1143 mm (45 inches).

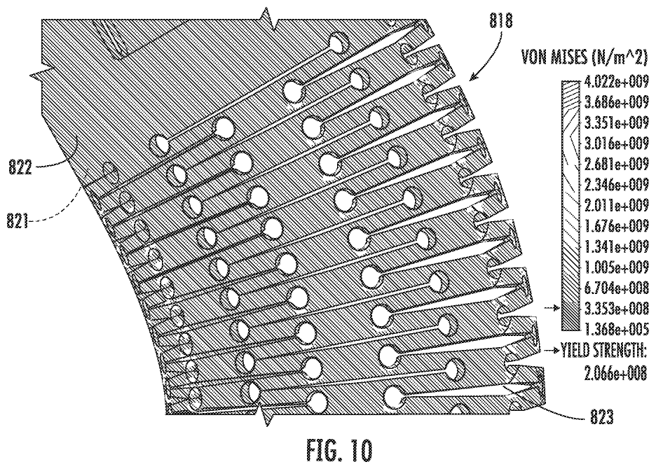

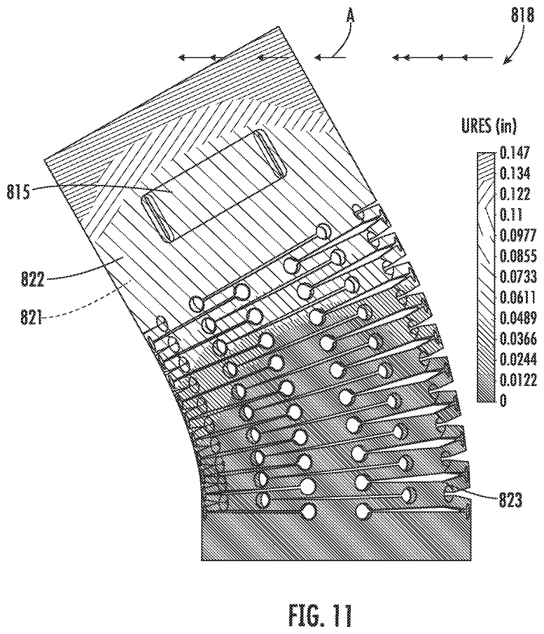

The hypotube 818 as described above provides a layer of the delivery catheter 800 that resists axial compression while allowing omnidirectional movement with respect to the central axis of the delivery catheter 800. The cut-outs 823 provide bending flexibility by collapsing and expanding as the hypotube 818 bends. This expansion and contraction of the cut-outs 823 is illustrated in as part of the delivery catheter 800 as shown in FIG. 10 and FIG. 11. FIG. 10 provides a side view of the hypotube bending in response to a radial force applied at/along the distal tip portion 805. FIG. 11 illustrates a finite element analysis rendering of the hypotube of FIG. 10 in response to a stress applied in the direction of the arrow A. The hypotube 818 further provides resistance to axial compression as the cut-outs are sized and configured having height that provides minimal axial compression of the cutouts when an axial force is applied.

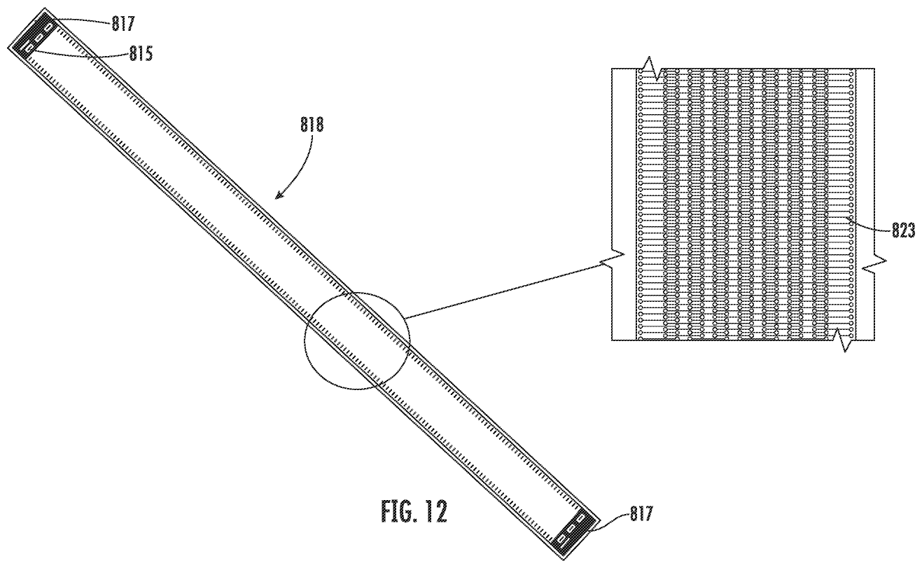

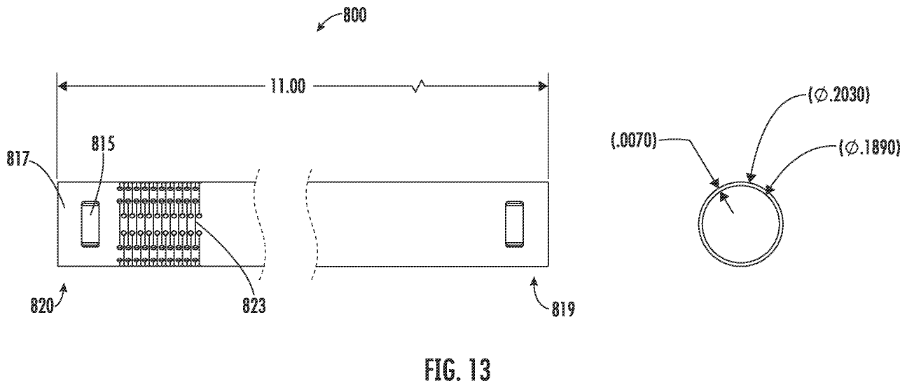

In the examples shown in FIGS. 10-13, each of the plurality of cut-outs 823 are disposed in circumferential rows along the hypotube 818 (e.g., along an axial length of the hypotube 818). FIG. 11 provides a flattened side view of the hypotube 818. As shown in FIG. 11, the hypotube 818 includes rows of cut-outs 823 along the majority of the axial length of the hypotube 818. But in other examples, the hypotube 818 includes rows of cut-outs 823 along only a portion of the axial length of the hypotube 818, shown, for example, in FIG. 13 illustrating a side and end view of an example hypotube 818.

As illustrated in FIG. 11 (and FIG. 13), the hypotube 818 includes end sections 817 and both the proximal end 819 and distal end 820. The cut-outs 823 are offset from the end sections 817 of the hypotube 818 to provide structural rigidity at the proximal end 819 and distal end 820 of the hypotube 818 by providing an offset/spacing between the end of the hypotube 818 and the first (proximal or distal) row of cut-outs 823. The first row of cut-outs 823 is offset from the proximal end 819 and/or distal end 820 of the hypotube 818 by about 3.81 mm (0.150 inches). The end sections 817 also include anchoring windows 815. The anchoring windows 815 provide cut-outs/openings in the end sections 817 that receive reflow material (e.g., reflowed outer jacket 808 material) during manufacturing. The reflow material embeds within the anchoring windows 815, and after cooling/hardening forms a mechanical interlock or connection between the hypotube 818 and the outer jacket 808. The hypotube 818 has a first distal most row of cut-outs.

In some examples, each row of cutouts includes at least two cut-outs or at least four cut-outs 823. As illustrated in the enlarged portion of FIG. 11, the example hypotube 818 includes four cut-outs 823 per row. The rows of cut-outs can be distributed about the circumference of the hypotube 818 in a way that fits the needs of the application. For example, some applications require even omnidirectional movement, while other applications require the delivery catheter 800 to have greater resistance to bending in a first direction than in a second direction. As such, in some examples, such as the examples shown in FIGS. 10-13 each of the plurality of cut-outs 823 in the same row are distributed symmetrically about the circumference of the hypotube 818. But, in other examples, each of the plurality of cut-outs 823 of in the same row are distributed asymmetrically about the circumference of the hypotube 818. As illustrated in FIG. 11, the plurality of cut-outs 823 in adjacent row are circumferentially offset from the plurality of cut-outs 823 in an adjacent row. This promotes even distribution of compressive resistance along the axial length of the hypotube 818.

In some examples, such as the examples shown in FIGS. 10-13 each of the plurality of cut-outs 823 includes a circumferential slit and at least one semicircular hole provided at the end of the slit. The circumferential hole provides stress relief during bending and prevents unwanted damage to the hypotube 818 (e.g., by the slit breaking and/or deforming circumferentially and/or axially in the body of the hypotube 818). As illustrated in FIGS. 10-13, each of the plurality of cut-outs 823 includes a circumferential slit and a semicircular hole provided at each circumferential end of the slit.

The semicircular holes in the examples shown in FIGS. 10-13, have a radius of about 0.1524 mm (0.006 inches). In other examples, the radius of the semicircular hole ranges between about 0.1016 mm (0.004 inches) and about 0.762 mm (0.030 inches). Each of the slits has a circumferential length of about 3.3 mm (0.130 inches). But in other examples, each of the slits have a circumferential length ranging between about 0.010 mm (0.0004 inches) and about 0.025 mm (0.0010 inches). Each of the slits has an axial width/height of about 0.018 mm (0.0007 inches). But in other examples, each of the slits has an axial width/height ranging between about 0.127 mm (0.0050 inches) and about 0.254 mm (0.0100 inches). The hypotube 818 has a thickness of about 0.0070 inches. But in other examples, the hypotube 818 has a thickness ranging between about 0.0050 inches and about 0.0100 inches.

The hypotube 818 has an outer diameter of about 0.2030 inches. But in some examples, the hypotube 818 has an outer diameter ranging between about 0.3000 inches and about 0.2000 inches. The hypotube 818 has an inner diameter of about 0.1890 inches. But in some examples, the hypotube 818 has an inner diameter ranging between about 0.1000 and about 0.2000 inches. In some examples, such as the example shown in FIG. 8 and FIG. 9 an overall length of the hypotube 818 is less than an overall length of the distal tip portion 805 of the delivery catheter 800. The overall length of the hypotube 818 ranges is about 11.0 inches. But, in some examples, an overall length of the hypotube 818 ranges between 10.5 inches and about 11.5 inches. The hypotube 818 in the example shown in FIGS. 8-13 is composed of stainless steel. But in some examples, the hypotube 818 is composed of at least one of a polymer, a metal, a composite.

As described above, the delivery catheter 800 includes a braided layer 826 which provides structural support for the delivery catheter 800. The braided layer 826 has a proximal end 830 and a distal end 832. The braided layer 826 includes a plurality of fibers 834 (e.g., wires) disposed in a braided pattern and defines a central lumen, an inner surface 838, and an outer surface 840. In the example shown in FIG. 8, the braided layer 826 extends along the entire length delivery catheter 800, i.e., along the body portion 803 and the distal tip portion 805 of the delivery catheter 800. But in other examples the braided layer 826 along only a portion of the catheter. In some examples, the braided layer 826 extends along all or a portion of the entire length of the body portion 803 of the delivery catheter 800. As illustrated in FIG. 9, the braided layer 826 extends along the entire length of the body portion 803 and partially into the distal tip portion 805 of the catheter.

Like the delivery catheters 300, 600, 700, the braided layer 826 is composed of a plurality of interwoven fibers 834. Each of the fibers 834 has a curvilinear cross section, although in other examples, the fibers 834 have a rectilinear cross section. The fibers 834 are composed of stainless steel, but in other examples, the fibers 834 are composed of polyester or at least one of a polymer, a metal, or a composite or any other material suitable for composing a stiffening braided layer 826 in a delivery catheter 800. Each of the plurality of interwoven fibers 834 has a diameter of about 0.004 inches. In other examples, each of the plurality of interwoven fibers 834 has a diameter ranging between about 0.002 inches and about 0.006 inches or between about 0.003 inches and about 0.005 inches. The interwoven fibers 834 can be oriented in a single strand braid pattern or in a double strand braid pattern where each interweave includes a plurality of parallel fibers 834 for each weave strand. The plurality of interwoven fibers 834 is composed of 16 individual fibers 834. But in other examples the plurality of interwoven fibers 834 is composed of about 10 to about 20 individual fibers 834. The braided layer 826 has a braid density which allows for omnidirectional bending with respect to the central axis of the delivery catheter 800. As the braid density increases, the braided layer 826 becomes more resistant to bending. In the example shown in FIG. 8, the braid density of the braided layer 826 is about 25 picks per inch. But in other examples, the braided layer 826 has a braid density between about 20 to about 30 picks per inch.

Similar to the catheter example described above, the delivery catheter 800 shown in FIG. 8 and FIG. 9 include at least one tensile stiffening fiber 827 having a proximal end 828 and a distal end 829. As described above, the tensile stiffening fiber 827 limits tensile deformation along the axial length of the delivery catheter 800 and limits bending of the delivery catheter 800 in a direction opposite the circumferential location of the at least one tensile stiffening fiber 827. For example, if the tensile stiffening fiber is located at a 90-degree position (e.g., 3 o'clock) around the circumference of the delivery catheter 800, the delivery catheter 800 will resist bending at the circumferentially 90-degree position (e.g., 3 o'clock).

The tensile stiffening fiber 827 extends along at least a portion of the entire length of the body portion 803 of the delivery catheter 800. In the example shown in FIG. 8 and FIG. 9 the tensile stiffening fiber 827 extends along an entire length the body portion 803 of the delivery catheter 800. But, in other examples, a length of the at least one tensile stiffening fiber 827 corresponds to a length of the distal tip portion 805 or is greater than the length of the distal tip portion 805. As described above with respect to delivery catheter 300, the tensile stiffening fiber 827 is coupled to the other components of the delivery catheter 800 by the weaving/intertwining the tensile stiffening fiber 827 into the braid pattern of the braided layer 826. The tensile stiffening fiber 827 is further secured to the braided layer 826 when the outer jacket 808 is reflowed into the braided layer 826. Like delivery catheter 300, the tensile stiffening fiber 627 is fixedly coupled to the proximal and distal ends of the delivery catheter 800. In the example shown in FIG. 8 and FIG. 9, the tensile stiffening fiber 867 is woven into the braided layer 826 along an entire length of the braided layer 826. But, in other examples, it is contemplated that the tensile stiffening fiber 827 is woven into a portion of the length of the braided layer 826. Similar to tensile stiffening fiber 327, The example shown in FIG. 8 includes Technora® Kevlar. But other examples include a tensile stiffening fiber 827 composed of other Kevlar, Liquid Crystal Polymer (LCP), a polyimide type polymer at least one of a polymer, a metal, a composite.

The examples shown in FIG. 8 and FIG. 9 include four tensile stiffening fibers 827 spaced around the circumference of the delivery catheter 800 by 90 degrees. But other examples include six or less, four or less, or one individual tensile stiffening fiber 827. In examples having a plurality of tensile stiffening fibers 827, the plurality of individual tensile stiffening fibers 827 can be equally spaced around the circumference of the delivery catheter 800 (e.g., every 60 degrees, 90 degrees, 120 degrees, 180 degrees), or irregularly spaced out. In examples having a one individual tensile stiffening fiber 827, the one individual tensile stiffening fiber 827 defines the neutral axis of the delivery catheter 800.

As described above, the delivery catheter 800 includes an outer jacket 808 which forms the outermost layer of the delivery catheter 800. The outer jacket 808 is composed of an elastic material Like delivery catheter 300, the elasticity of the outer jacket 808 varies (longitudinally) along a length of the outer jacket 808. In the examples shown in FIG. 8 and FIG. 9, the elasticity of the outer jacket 808 increases between the proximal end 809 and distal end 811 of the outer jacket 808 such that the distal end 806 (e.g., along the distal tip portion 805) of the delivery catheter 800 more easily bends to conform to the aortic geometry of a patient Like delivery catheter 300, the durometer of the outer jacket 808 decreases between the proximal end 804 and the distal end 806 of the delivery catheter 800. However, as described above, the distal most and proximal most segments of the delivery catheter 800 is constructed from a material having a higher durometer (and lower elasticity) than the next adjacent segment to facilitate coupling of the tensile stiffening element 827 to the proximal and distal ends 804, 806 of the delivery catheter 800.

Like delivery catheter 300, the delivery catheter 800 in the examples shown in FIG. 8 and FIG. 9 includes a tie layer 831. The tie layer 831 is provided on an inner surface of the hypotube 818 and creates a surface for improved adhesion between the hypotube 818 and the liner 833, described below. The tie layer 831 bonds the liner 833 to the outer jacket 808. The tie layer 831 is reflowed and/or bonded with the outer jacket 808. The tie layer does not bond to the hypotube 818, instead it encapsulates the hypotube 818 preventing material from entering between cut-outs.

As illustrated in FIG. 8 and FIG. 9, the tie layer 331 extends along the entire axial length of the inner surface of the hypotube 818. In other examples, the tie layer 831 extends along a portion of the axial length of the hypotube 818. The tie layer 831 has a thickness of about 0.003 inches, but in other examples the tie layer 331 has a thickness ranging between about 0.002 inches and about 0.005 inches. The tie layer 331 is composed of a polyether block amide material such as Pebax, and/or a polyamide (e.g., Vestamid®), polyamide 12 (e.g., Rilsamid®), or any other suitable material.

As described above, the delivery catheter 800 includes a liner 833 provided on an inner surface of the tie layer 831. The inner surface of the liner 833 defines an inner lumen of the delivery catheter 800. The liner 833 extends along an entire length of the inner surface of the tie layer 831 or the inner lumen of the delivery catheter 800. In some examples, the liner 833 has a length that is greater than a length of the distal tip portion 805. In some examples, the liner 833 extends along at a portion of the entire length of the body portion 803 of the delivery catheter 800 or the entire length of the body portion 803 of the delivery catheter 800. The liner 833 in the example shown in FIG. 8 has a thickness of about 0.002 inches. But in other examples, the liner 833 has a thickness ranging between 0.001 inches and about 0.004 inches or between 0.0015 inches and about 0.003 inches. The liner 833 is composed of polytetrafluoroethylene (PTFE), PA12 materials, polyether block amide (PEBA), fluorinated ethylene propylene (FEP), or any other polymer suitable for forming an inner most layer of a flexible delivery catheter 800.

Also disclosed herein is a method of making a flexible delivery catheter 300. Similar method is contemplated for the construction of flexible delivery catheters 600, 700, 800. The method includes forming an outer jacket 308, a coil layer 318 and a braided layer 326 as described above and shown in FIG. 3 and FIG. 4. These components can be formed by molding, extrusion, or any other method suitable to form insertable medical catheter components. The coil layer 318 is disposed within the central lumen of the braid layer. The braided layer is disposed between the outer jacket 308, and the coil layer 318 and the outer jacket 308 is disposed on the outside of the coil layer 318 and the braided layer. Each of these components are coupled together and reflowed together to form a single catheter. In some examples, the coil layer 318 includes a gap/spacing between adjacent turns of the coil winding 320. In such examples, a PET layer as described above can be placed on either side of the coil layer 318 to prevent debris from lodging within the gap/spacing during the reflow process.

In examples wherein the outer jacket 308 includes a plurality of longitudinal segments as described above, each of the plurality of longitudinal segments are reflowed together with at least one an adjacent longitudinal segment having a differing elastic property. In examples that include tensile stiffening fibers 327, the tensile stiffening fibers 327 are coupled to the braided layer by weaving the tensile stiffening fiber 327 at least partially into the braided layer. The tensile stiffening fiber 327 is inserted into the distal tip of the outer jacket 308 and securing it thereto (e.g., via reflow and/or heat processing).

Also disclosed herein is a method of making a flexible delivery catheter 800 including a hypotube 818. The method includes forming an outer jacket 808, the hypotube 818 and a braided layer 826 as described above and shown in FIGS. 8-13. These components can be formed by molding, extrusion, or any other method suitable to form insertable medical catheter components. The hypotube 818 is disposed within the inner lumen of the braided layer 826. The braided layer 826 is disposed between the outer jacket 808 and the hypotube 818 such that the outer jacket 808 is disposed on the outside of the hypotube 818 and the braided layer 826. In another example, the hypotube is disposed within the outer jacket 808 along the distal tip portion 805 if of delivery catheter 300 and the braided layer 826 is disposed within the outer jacket along the body portion 803 of the catheter. The distal end of the braided layer can be provided either within the central lumen of the hypotube 818 and/or on the outer surface of the hypotube 818. Each of these components are coupled together and reflowed together to form a single delivery catheter 800.

In examples wherein the outer jacket 808 comprises a plurality of longitudinal segments as described above of varying durometer, each of the plurality of longitudinal segments are reflowed together with at least one an adjacent longitudinal segment having a differing elastic property. In examples that include tensile stiffening fibers 827, the tensile stiffening fibers 827 are coupled to the braided layer 826 by weaving the tensile stiffening fiber 827 at least partially into the braided layer 826. In other examples, the tensile stiffening fibers 827 are coupled to the hypotube 818 by weaving the tensile stiffening fiber 827 at least partially into the cut-outs 823 and/or the anchoring windows 815. In another example, the tensile stiffening fiber 827 is inserted into the distal tip of the outer jacket 808 and secured thereto.

FIGS. 20A and 20B show another example of a flexible delivery catheter 1100 with a hypotube 1118 rather than a coil layer. The delivery catheter 1100 includes similar structures and features to delivery catheter 900 as described above, the differences will be discussed in more detail below, like element numbers will be used to identify like elements. Similar to delivery catheter 900, flexible delivery catheter 1100 includes an outer jacket 1108 having various elasticity characteristics along its axial length, a hypotube 1118 rather than a coil layer, a braided layer 1126 which provides structural rigidity, a tensile stiffening fiber 1127 which provides tensile stiffness, a delivery capsule 1150 which houses a medical device, and a coupler 1160 which joins the delivery capsule 1150 and the delivery catheter 1100 keeping the hypotube 1118 disposed within the outer jacket 1108. The delivery catheter 1100 has a proximal end 1104 and a distal end 1106, an inner surface 1107, and an outer surface 1110. The delivery catheter 1100 defines an elongated tubular structure with a central lumen 1102 extending longitudinally between the proximal end 1104 and the distal end 1106. The central lumen 1102 is configured for passage of medical devices such as prosthetic heart valves therethrough. The delivery catheter 1100 includes an elongated body portion 1103 and a distal tip portion 1105. The elongated body portion 1103 extends from the distal tip portion 1105 (at the distal end 1106 of the delivery catheter 1100) to the proximal end 1104 of the delivery catheter 1100. The elongated body portion 1103 provides a section of the delivery catheter 1100 that is rigid enough for effective manipulation by a physician (e.g., advancement through the patient's vasculature). The delivery catheter 1100 is omnidirectionally flexible and configured to bend in any direction away from a longitudinal axis 1101 of the delivery catheter 1100 while retaining compressive and tensile stiffness. As such, the flexible delivery catheter 1100 conforms to a patient's vascular structure and during placement and delivery of a medical device (e.g., prosthetic heart valve), while limiting axial deformation of the delivery catheter 1100 that can damage a patient's vascular structure.

The delivery catheter 1100 includes an elongated body portion 1103 and a distal tip portion 1105. The elongated body portion 1103 extends from the distal tip portion 1105 to the proximal end 1104 end of the delivery catheter 1100. The distal tip portion 1105 extends along a length of the delivery catheter 1100 from the distal end 1106 of the delivery catheter 1100 to the elongated body portion 1103. In the example shown in FIGS. 20A and 20B, as discussed further below, the distal tip portion 1105 corresponds to the axial location of the cavity/second outer jacket portion 1016, and the elongated body portion 1103 corresponds to the first outer jacket portion 1115. Also, in some examples, the outer diameter of the distal tip portion 1105 corresponds with the outer diameter of the elongated body portion 1103, such that a smooth transition is formed along the outer surface of the delivery catheter 1100 between the elongated body portion 1103 and distal tip portion 1105. In other examples, the outer diameter of the distal tip portion 1105 is greater than the outer diameter of the elongated body portion 1103, such that the outer diameter of the delivery catheter 1100 increases from the elongated body portion 1103 to the distal tip portion 1105 to the delivery capsule 1150. In this example, the outer diameter of the elongated body portion 1103 may or may not taper radially outwardly to the outer diameter of the distal tip portion 1103. In further alternative examples, the outer diameter of the distal tip portion 1105 is less than the outer diameter of the elongated body portion 1103.

In the example shown in FIGS. 20A and 20B, the outer diameter of the distal tip portion 1105 and elongated body portion 1103 is about 5.4 mm But in other examples, the outer diameter of the body portion 1103 ranges between about 4.5 mm and about 6.0 mm or between about 5.0 mm and about 6.0 mm. In other examples, the outer diameter of the distal tip portion 1105 ranges between about 4.5 mm and about 6.0 mm, or between about 5.0 mm and about 6.0 mm.

As described above, the delivery catheter 1100 includes an outer jacket 1108, which forms the outermost layer of the delivery catheter 1100. The outer jacket 1108 has a proximal end 1109, a distal end 1111, an inner surface 1112, and an outer surface 1113. The outer jacket 1108 defines a central lumen 1114 extending longitudinally between the proximal end 1109 and the distal end 1111 and is composed of an elastic material. The outer jacket 1108 further includes a first outer jacket portion 1115 and a second outer jacket portion 1116. The first outer jacket portion 1115 is adjacent the proximal end 1109 of the outer jacket 1108, extends along at least a portion of the entire length of the body portion 1103, and has a first inner diameter within the central lumen 1114 of the outer jacket 1108. The second outer jacket portion 1116 is adjacent the distal end 1111 of the outer jacket 1108, extends along at least a portion of the distal tip portion 1105. The second outer jacket portion 1116 has a second inner diameter within the central lumen 1114 of the outer jacket 1108. In the example illustrated in FIG. 20A, the first inner diameter is less than the second inner diameter such that the change in inner diameter between the first inner diameter and the second inner diameter creates a shoulder 1117 between the first outer jacket portion 1115 and the second outer jacket portion 1116. The shoulder 1117 of the outer jacket 1108 further includes at least one axially extending recess 1119 that is corresponds to an at least one axially extending protrusion 1121 that extends from the proximal end of the hypotube 1118 as discussed further below. In the example shown in FIGS. 20A and 20B, the shoulder 1117 of the outer jacket 1108 includes two axially extending recesses 1119, but, in other examples, the shoulder 1117 of the outer jacket 1108 includes more than two axially extending recesses, one axially extending recess, or no axially extending recesses.

Also, in the example shown in FIGS. 20A and 20B, the first inner diameter of the first outer jacket portion 1115 is about 4.8 mm. In other examples, the first inner diameter of the first outer jacket portion 1115 ranges between about 3.5 mm and about 5 mm or between 4.0 mm and about 4.5 mm. The second inner diameter of the second outer jacket portion 1116 shown in FIGS. 20A and 20B is 5.16 mm. In further examples, the second inner diameter of the second outer jacket portion 1116 ranges between about 4.6 mm to about 5.4 mm. Also, in the example shown in FIGS. 20A and 20B, the axial location of the second outer jacket portion 1116 corresponds, at least in part, to location of the distal tip portion 1105 of the delivery catheter 1100.

The length of the second outer jacket portion 1116 is less than the length of the first outer jacket portion 1115 of the delivery catheter 1100. In the example shown in FIGS. 18A and 18B, the overall length of the outer jacket 1108 is 1320.8 mm (52 inches), but in other examples, the overall length of the outer jacket 1108 ranges between about 762 mm (30 inches) to about 1178 mm (70 inches) or between 889 mm (35 inches) to 1397 mm (55 inches).

Like delivery catheter 900 and other catheters described herein, the elasticity of the outer jacket 1108 varies (longitudinally) along a length of the outer jacket 1108. In the example of FIGS. 20A and 20B, the elasticity of the outer jacket 1108 varies between the proximal end 1109 and distal end 1111 of the outer jacket 1108 such that the distal end 1106 (e.g., along distal tip portion 1105) of the delivery catheter 1100 more easily bends to conform to the aortic geometry of a patient Like delivery catheter 900, the durometer of the outer jacket 1108 decreases between the proximal end 1104 and the distal end 1106 of the delivery catheter 1100. The outer jacket includes various longitudinal segments (e.g., segments 1108a-1108d) which are constructed from materials having varying durometer. The materials of the various longitudinal segments 1108a-1108d (with varying durometers) are reflowed together to form a single outer jacket 1108.

In the example shown in FIGS. 20A and 20B, the outer jacket 1108 is composed of Pebax. But, in other examples, at least a portion of the outer jacket 1108 comprises another polyether block amide, another elastomer, a polyimide (e.g., Vestamid®), or any other material suitable for forming the outermost layer of a flexible catheter.

The coupler 1160 as shown in FIGS. 22A-22F joins the delivery catheter 1100 to the delivery capsule 1150. The coupler 1160 has a body with a proximal end 1162 and a distal end 1164, an outer surface 1166, and an inner surface 1168. The inner surface 1168 of the coupler 1160 defines a central lumen 1170 that extends longitudinally between the proximal end 1162 and the distal end 1164 of the coupler 1160. The central lumen 1170 of the coupler 1160 includes a cylindrical portion 1172 adjacent the proximal end 1162 of the coupler 1160, an increasing taper portion 1174 adjacent the distal end 1164 of the coupler 1160. A radially extending protrusion/shoulder 1176 is provided between the cylindrical portion 1172 and the increasing taper portion 1174. As illustrated in FIGS. 22D, 22F and 22G, the diameter of the radially extending protrusion/shoulder 1176 is less than the diameter of the cylindrical portion 1172. Furthermore, the radially extending protrusion/shoulder 1176 includes two axially extending recesses 1177 that correspond in size and shape to the two axially extending protrusions 1125 on the distal end 1124 of the hypotube. In other examples, the radially extending protrusion/shoulder 1176 of the coupler 1160 includes more than two axially extending recesses, one axially extending recess, or no axially extending recesses. It is further contemplated that the radially extending protrusion/shoulder 1176 of the coupler 1160 can include a tapered edge.

As illustrated in FIGS. 22D and 22F, the central lumen of the coupler 1160 also includes at least one rib 1173 projecting inward from the inner surface of the cylindrical portion 1172. The ribs 973 helps improve purchase/engagement between the coupler 960 and the delivery catheter 900. In one example, the ribs 1173 form a generally rectangular shape extending along a portion of the cylindrical portion 1172. The rib 1173 includes a tapered proximal edge to help ease introduction of the delivery catheter 1100 into the central lumen of the coupler 1160.

The outer surface 1166 of the coupler 1160 includes a recess 1178 that extends around the entire circumference of the outer surface 1166 of the coupler 1160 and a decreasing taper 1180 that starts between the recess 1178 and proximal end 1162 of the coupler 1160 and extends axially and radially inwardly towards the proximal end 1162 of the coupler 1160. The distal end of the taper 1180 has a generally cylindrical shape and a diameter greater than the diameter of the recess 1178 (e.g., cylindrical portion). The recess 1178 on the outer surface 1166 of the coupler 1160 is configured to receive the delivery capsule 1150 as discussed below. The recess 1178 includes a reduced diameter cylindrical portion that abuts a shoulder formed between the recess 1178 and the tapered portion. In one example, at least one rib 1179 projecting outward from the outer surface of the cylindrical portion of the recess 1178. The ribs 1179 help improve purchase/engagement between the coupler 1160 and the delivery capsule 1150. As illustrated in FIGS. 22B-22F, the ribs 1179 form a generally rectangular shape that extends along a portion of the recess 1178. The ribs 1179 include a tapered distal edge to help ease introduction of the delivery capsule onto the recess 1178 of the coupler 1160.

When assembled, the proximal end 1162 of the coupler 1160 is disposed adjacent the distal end 1111 of the outer jacket 1108 such that the radially extending protrusion/shoulder 1176 on the inner surface 1168 of the coupler 1160 abuts the distal end 1111 of the outer jacket 1108. The radially extending protrusion/shoulder 1176 with the two axially extending recesses 1177 on the inner surface 1168 of the coupler 1160 and the second outer jacket portion 1116 form a cavity 1182 that the hypotube 1118 is disposed within. In some examples, the inner diameter of the radially extending protrusion/shoulder 1176 formed in the central lumen of the outer jacket 1108 corresponds to the inner diameter of the central lumen 1170 of the hypotube 1118.

When the delivery catheter 1100 is assembled, the distal end 1164 of the coupler 1160 is disposed adjacent a delivery capsule 1150 such that the recess 1178 on the outer surface 1166 of the coupler 1160 receives the delivery capsule 1150. The central lumen 1170 of the delivery capsule 1150 is sized and configured to receive a prosthetic heart valve in a crimped or non-expanded configuration. In the example shown in FIGS. 18A and 18B, the outer diameter of the delivery capsule 1150 is 7.9 mm, but in other examples, the outer diameter of the delivery capsule 1150 ranges from about 7 mm to about 9 mm. The outer diameter of the delivery capsule 1150 corresponds to the outer diameter of the coupler 1160 and is greater than the outer diameter of the outer jacket 1108.

It is contemplated that the coupler 1160 can be coupled to the delivery capsule 1150 by at least one of a press fit, an interference fit, a snap fit, a mechanical fastener, a weld, and an adhesive and that the coupler 1160 can be coupled to the outer jacket 1108 by at least one of a press fit, an interference fit, a snap fit, a mechanical fastener, a weld, and an adhesive.

The hypotube 1118 resists axial compression and tension applied to the delivery catheter 1100, while facilitating bending of the delivery catheter 1100 in a direction away from the longitudinal axis of the delivery catheter 1100. The hypotube 1118 is substantially similar to the hypotube 818 as described above and has a proximal end 1122, a distal end 1124, an inner surface, and an outer surface. The hypotube 1118 further includes two axially extending proximal end protrusions 1121 and two axially extending distal end protrusions 1125. Both the proximal end protrusions 1121 and the distal end protrusions 1125 are equally spaced from each other, but in other examples, the proximal and distal end protrusions are unequally spaced from each other. In other examples, the hypotube 1118 includes multiple, one, or no axially extending proximal end protrusions and/or axially extending distal end protrusions. The hypotube 1118 provides compressive stiffness to the delivery catheter 1100 during axial compressive loads.

The hypotube 1118 is disposed within the cavity 1182 formed between the outer jacket 1108 and the coupler 1160 such that the outer surface of the hypotube 1118 abuts the inner surface 1112 of the second outer jacket portion 1116, the two axially extending proximal end protrusions 1121 of the hypotube 1118 are disposed in the two axially extending recesses 1119 of the shoulder 1117 of the outer jacket 1108, and the two axially extending distal end protrusions 1125 of the hypotube 1118 are disposed in the two axially extending recesses 1177 of the coupler 1160. In some examples, the inner diameter of the hypotube 1118 is equal to the first inner diameter of the outer jacket 1108 at the location corresponding to the first outer jacket portion 1115, and the outer diameter of the hypotube 1118 is less than the second inner diameter of the outer jacket 1108 at the location corresponding to the second outer jacket portion 1116. In other examples, the inner diameter of the hypotube 1118 is greater than the first inner diameter of the first outer jacket portion 1115. The outer diameter of the hypotube 1118 corresponds with the second inner diameter of the second outer jacket portion 1116 and is greater than the first inner diameter of the first outer jacket portion 1115. It is also contemplated that the inner diameter of the hypotube 1118 is greater than the first inner diameter of the first outer jacket portion 1115. In the example shown in FIGS. 20A and 20B, the inner diameter of the hypotube 1118 is about 0.1890 inches and the outer diameter of the hypotube 1118 is about 0.2030 inches. In other examples, the hypotube 1118 has an inner diameter ranging from about 0.1000 inches to about 0.2000 inches. Also, in other examples, the hypotube 1118 has an outer diameter ranging from about 0.2000 inches to about 0.3000 inches.

In the example shown in FIGS. 20A and 20B, the hypotube 1118 has a length that is equal to the length of the distal tip portion 1105 and equal to the length of the cavity 1182 (not including the two axially extending proximal end protrusions 1121 and the two axially extending distal end protrusions 1125). In some examples, the length of the hypotube 1118 corresponds to the length of the distal tip portion 1105 and/or the length of the cavity 1182. But in other examples, a length of the hypotube 1118 is less than the length of the distal tip portion 1105 and/or cavity 1182. In further examples, the length of the hypotube 1118 is greater than a length of the distal tip portion 1105 and/or cavity 1182 such that the hypotube 1118 extends into the body portion 1103 of the delivery catheter 1100.

Also, in the example shown in FIGS. 18A and 18B, the hypotube 1118, which is measured in a direction along the longitudinal axis 1101 of the delivery catheter 1100, is about 215.9 mm (8.5 inches). In some examples, the hypotube 1118 has a length less than 305 mm (12 inches), less than 254 mm (10 inches), between 76 mm (3 inches) and 254 mm (10 inches), or between 178 mm (7 inches) and 254 mm (10 inches).

Similar to the other example hypotubes described herein, the outer surface of the hypotube 1118 further includes cut-outs 1123 to provide bending flexibility by collapsing and expanding as the hypotube 1118 bends similar to the cutouts 823 shown in FIGS. 10 and 11. The hypotube 1118 further provides resistance to axial compression as the cut-outs 1123 are sized and configured to have a height that provides minimal axial compression of the cut-outs 1123 when an axial force is applied.

In the example shown in FIGS. 20A and 20B, each of the plurality of cut-outs 1123 are disposed in circumferential rows along the hypotube 1118 (e.g., along an axial length of the hypotube 1118). The hypotube 1118 includes rows of cut-outs 1123 along the majority of the axial length of the hypotube 1118. But in other examples, the hypotube 1118 includes rows of cut-outs 1123 along only a portion of the axial length of the hypotube 1118.

The cut-outs 1123 are offset from the proximal end 1122 and distal end 1124 of the hypotube 1118 to provide structural rigidity at the proximal end 1122 and distal end 1124 of the hypotube 1118 by providing an offset/spacing between the ends of the hypotube 1118 and the first (proximal or distal) row of cut-outs 1123. The first row of cut-outs 1123 is offset from the proximal and/or distal end 122, 124 of the hypotube 1118 by about 3.81 mm (0.150 inches). In other examples, the first row of cut-outs is offset from the proximal and/or distal ends of the hypotube by about 0.05 inches to 0.25 inches.

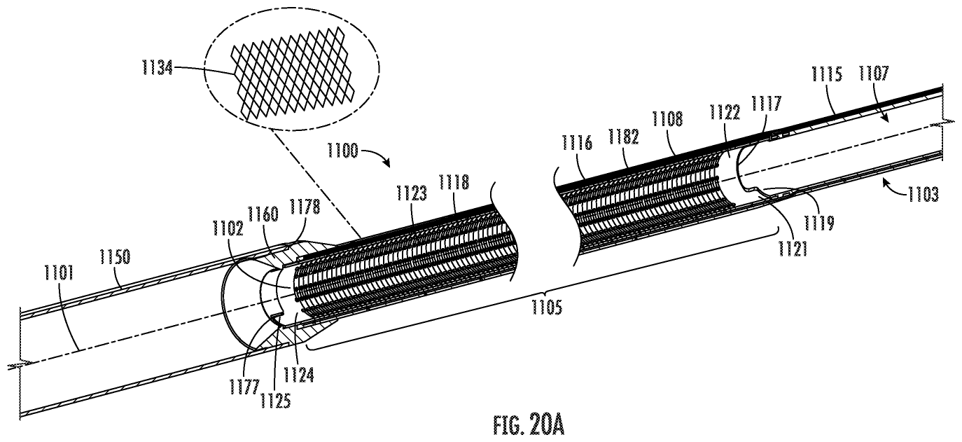

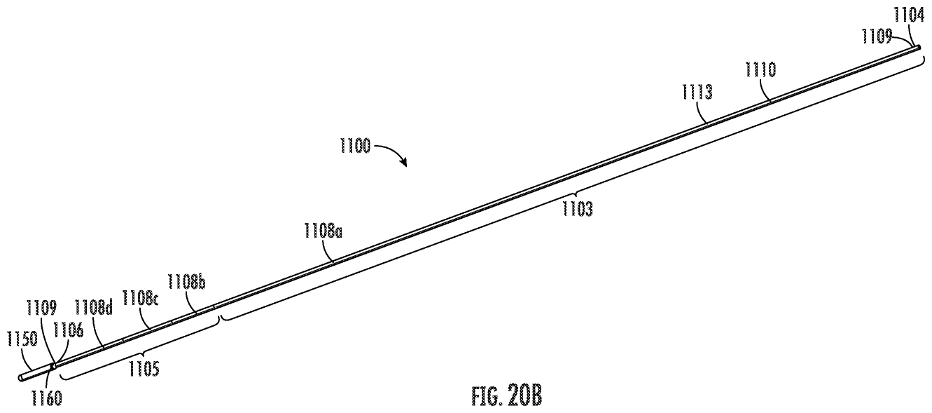

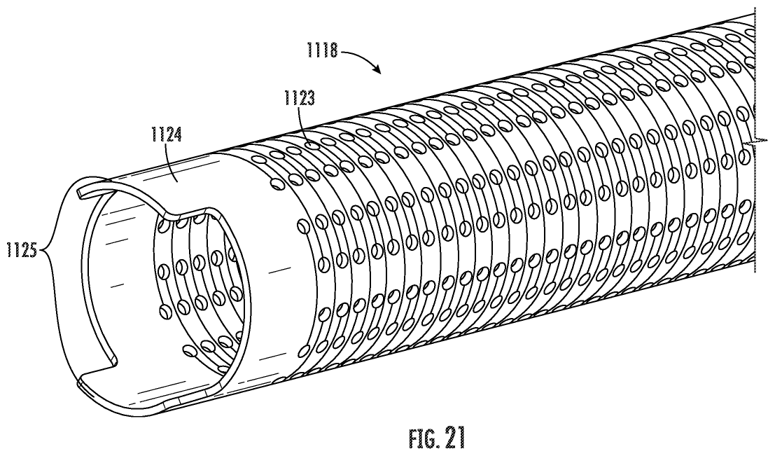

In some examples, each row of cutouts includes at least two cut-outs or at least four cut-outs. As illustrated in the enlarged portion of FIG. 21, the example hypotube 1118 includes four cut-outs 1123 per row. The rows of cut-outs 1123 can be distributed about the circumference of the hypotube 1118 in a way that fits the needs of the application. For example, some applications require even omnidirectional movement, while other applications require the catheter to have greater resistance to bending in a first direction than in a second direction. As such, in some examples, such as the examples shown in FIGS. 20A-21, 23A and 23B each of the plurality of cut-outs 1123 in the same row are distributed symmetrically about the circumference of the hypotube 1118. But, in other examples, each of the plurality of cut-outs 1123 of in the same row are distributed asymmetrically about the circumference of the hypotube 1118. As illustrated in FIG. 21, the plurality of cut-outs 1123 in adjacent row are circumferentially offset from the plurality of cut-outs 1123 in an adjacent row. This promotes even distribution of compressive resistance along the axial length of the hypotube 1118.

In some examples, such as the examples shown in 20A-21, 23A and 23B each of the plurality of cut-outs 1123 includes a circumferential slit and at least one semicircular hole provided at the end of the slit. The circumferential hole provides stress relief during bending and prevents unwanted damage to the hypotube 1118 (e.g., by the slit breaking and/or deforming circumferentially and/or axially in the body of the hypotube 1118). Each of the plurality of cut-outs 1123 includes a circumferential slit and a semicircular hole provided at each circumferential end of the slit.

The semicircular holes in the examples shown in FIGS. 20A-21, 23A and 23B, have a radius of about 0.1524 mm (0.006 inches). In other examples, the radius of the semicircular hole ranges between about 0.1016 mm (0.004 inches) and about 0.762 mm (0.030 inches). Each of the slits has a circumferential length of about 3.3 mm (0.130 inches). But in other examples, each of the slits have a circumferential length ranging between about 0.010 mm (0.0004 inches) and about 0.025 mm (0.0010 inches). Each of the slits has an axial width/height of about 0.018 mm (0.0007 inches). But in other examples, each of the slits has an axial width/height ranging between about 0.127 mm (0.0050 inches) and about 0.254 mm (0.0100 inches). The hypotube 1118 has a thickness of about 0.0070 inches. But in other examples, the hypotube 1118 has a thickness ranging between about 0.0030 inches and about 0.0150 inches.

The hypotube 1118 in the example shown in FIGS. 20A-21, 23A and 23B is composed of stainless steel. But in some examples, the hypotube 1118 is composed of at least one of a polymer, a metal, a composite.

As described above the delivery catheter 1100 includes a braided layer 1126 which provides structural support for the delivery catheter 1100 such that the distal tip portion 1105 will not deform when receiving axial load from the hypotube 1118. The braided layer 1126 has a proximal end 1130 and a distal end 1132. The braided layer 1126 includes a plurality of fibers 1134 interwoven into a braided pattern and defines a central lumen, an inner surface 1138, and an outer surface 1140. In the example shown in FIGS. 20A and 20B, the braided layer 1126 extends along the entire length of the outer jacket of the delivery catheter 1100. In another example, the braided layer extends along an entire length of the distal tip portion and then extends down into the first outer jacket portion towards the central lumen of the outer jacket. In yet another example, the braided layer extends along at least a portion of the distal tip portion and/or at least a portion of the elongated body such that the braided layer extends over a corresponding length of the coil layer but not the entire length of the outer jacket. In the example shown in FIGS. 20A and 20B, the braided layer 1126 extends along the entire length of the delivery catheter 900. As such, braided layer 926 has a length that is greater than an overall length of the hypotube 1118. But, in some implementations the braided layer 1126 and the hypotube 1118 are about the same length as measured along the axis 1101 of the delivery catheter 1100.

As described above, the braided layer 1126 is composed of a plurality of interwoven fibers 1134. Each of the fibers 1134 has a curvilinear cross section, although in other examples, the fibers 1134 have a rectilinear cross section. The fibers 1134 are composed of a liquid crystal polymer, but in other examples, the fibers 1134 are composed of at least one of a polyester, a polymer, a metal (e.g., stainless steel), a composite, Kevlar (e.g., Technora®), or any other material suitable for composing a stiffening braided layer 1126 in a delivery catheter 1100. Each of the plurality of interwoven fibers 1134 has a diameter of about 0.004 inches. In other examples, each of the plurality of interwoven fibers 1134 has a diameter ranging between about 0.002 inches and about 0.006 inches or between about 0.003 inches and about 0.005 inches. The interwoven fibers 1134 can be oriented in a single strand braid pattern or in a double strand braid pattern where each interweave includes a plurality of parallel fibers 1134 for each weave strand. The plurality of interwoven fibers 1134 is composed of 16 individual fibers 1134. But in other examples the plurality of interwoven fibers 1134 is composed of about 10 to about 20 individual fibers 1134. The braided layer 1126 has a braid density which allows for omnidirectional bending with respect to the central axis of the delivery catheter 1100. As the braid density increases, the braided layer 1126 becomes more resistant to bending. In the example shown in FIGS. 20A and 20B, the braid density of the braided layer 1126 is about 25 picks per inch. But in other examples, the braided layer 1126 has a braid density between about 20 to about 30 picks per inch.

As described above, the delivery catheter 1100 as shown in FIGS. 20A and 20B includes at least one tensile stiffening fiber 1127 having a proximal end 1128 and a distal end 1129. The tensile stiffening fiber 1127 limits tensile deformation along the axial length of the delivery catheter 1100 such that the bending of the delivery catheter 1100 is limited at a location/along the side corresponding to the circumferential location of the tensile stiffening fiber 1127. For example, if the tensile stiffening fiber is located at a 90-degree position (e.g., 3 o'clock) around the circumference of the delivery catheter 1100, the delivery catheter 1100 will resist bending at the circumferentially 90-degree position (e.g., 3 o'clock). Generally, the location of the tensile stiffening fiber 1127 determines the neutral bending axis of the delivery catheter 1100. That is, the stiffness and relative location of the tensile stiffening fiber 1127 (or location of multiple fibers with respect to each other) defines the line or plane along the delivery catheter 1100 at which no extension or compression occurs when the catheter is bent. For example, when the delivery catheter 1100 includes a single tensile stiffening fiber 1127, the neutral axis of the delivery catheter is defined by the axis of the tensile stiffening fiber 1127.

The tensile stiffening fiber 1127 extends along at least a portion of the entire length of the body portion 1103 of the delivery catheter 1100. In the example shown in FIGS. 20A and 20B, the least one tensile stiffening fiber 1127 extends along an entire length the body portion 1103 of the delivery catheter 1100. But, in other examples, a length of the at least one tensile stiffening fiber 1127 corresponds to a length of the distal tip portion 1105 or is greater than the length of the distal tip portion 1105.

The tensile stiffening fiber 1127 is coupled to the delivery catheter 1100 by the weaving/intertwining the tensile stiffening fiber 1127 within the fibers/braid pattern of the braided layer 1126. The tensile stiffening fiber 1127 is further secured to the braided layer 1126 when the outer jacket 1108 is reflowed into the braided layer 1126 during manufacturing. As previously discusses, the distal end of the delivery catheter 1100 includes materials of varying durometer along the length of the outer jacket 1108. A length/segment of the delivery catheter 1100 proximate the distal end 1106 of the distal tip portion 1105 comprises a material having a higher durometer than the next adjacent segment of the delivery catheter 1100. Reflow of the tensile stiffening fiber 1127 with the higher durometer material at the distal end 1106 of the distal tip portion 1105, results in higher friction between the tensile stiffening fiber 1127 and the higher durometer material of the outer jacket 1108, thereby preventing axial slippage/movement of the tensile stiffening fiber 1127 at the distal end of the catheter. Similarly, the proximal end of the delivery catheter 1100 includes a length/segment of material having a higher durometer than the next adjacent segment of the delivery catheter 1100. Accordingly, reflow of the tensile stiffening fiber 1127 with the higher durometer material at the proximal end 1104 of the delivery catheter 1100 prevents slippage/movement of the tensile stiffening fiber 1127 with respect to the end of the delivery catheter 1100. As such, the tensile stiffening fiber 1127 is secured both along the length of the fiber and at its proximal and distal ends, i.e., the tensile stiffening fiber 1127 is fixedly coupled to the proximal and distal ends of the outer jacket 1108/braided layer 1126 and along the along the braided layer 1126.

As shown in FIGS. 20A and 20B, the tensile stiffening fiber 1127 is woven into the braided layer 1126 along an entire length of the braided layer 1126. But, in other examples, it is contemplated that the tensile stiffening fiber 1127 is woven into a portion of the length of the braided layer 1126. For example, the at least one tensile stiffening fiber 1127 may not be woven into a first portion of the braided layer 1126 extending along the distal tip portion 1105 of the delivery catheter 1100 corresponding to the length of the hypotube but will be woven into a second portion of the braided layer 1126 extending along the elongated body portion 1103 of the delivery catheter 1100 between the distal tip portion 1105 and the proximal end 1104 of the delivery catheter 1100.

The example shown in FIGS. 20A and 20B includes Technora® Kevlar. But other examples, a tensile stiffening fiber 1127 is composed of at least one of a polymer, a metal, a composite including a polyimide type polymer, Kevlar, or Liquid Crystal Polymer (LCP).

The example shown in FIGS. 20A and 20B includes four tensile stiffening fibers 1127 spaced around the circumference of the delivery catheter 1100 by 90 degrees. But other examples include six or less, four or less, two or less, or one individual tensile stiffening fiber 1127. In examples having a plurality of tensile stiffening fibers 1127, the plurality of individual tensile stiffening fibers 1127 can be equally spaced around the circumference of the delivery catheter 900 (e.g., every 60 degrees, 90 degrees, 120 degrees, 180 degrees), or irregularly spaced around the circumference of the delivery catheter 1100. In examples having a one individual tensile stiffening fiber 1127, the one individual tensile stiffening fiber 1127 is located at a neutral axis of the delivery catheter 1100.

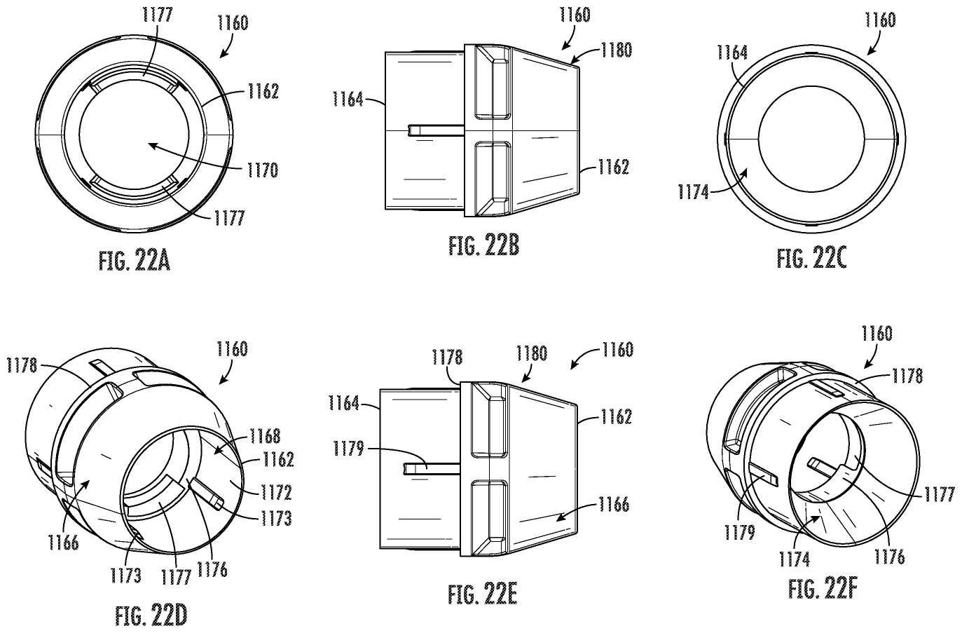

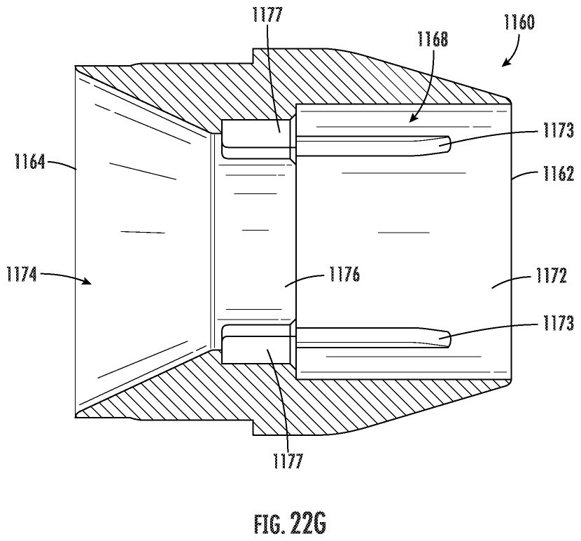

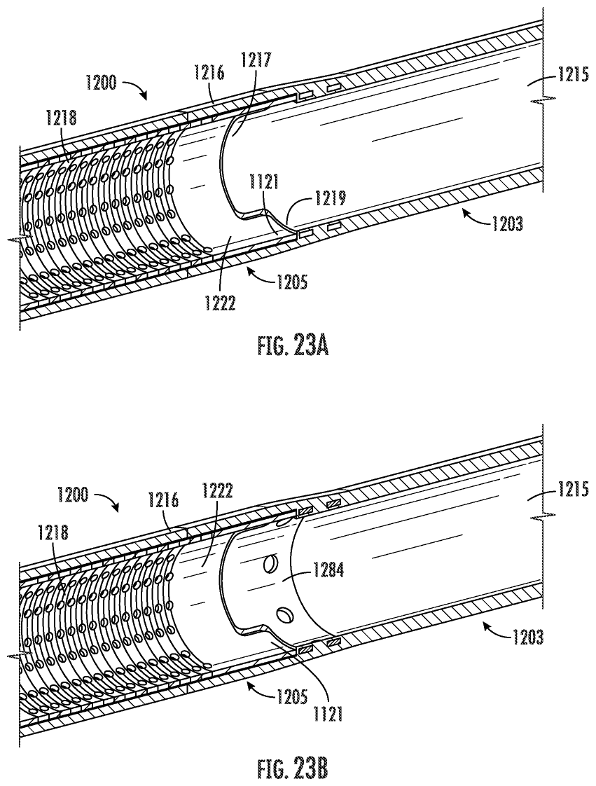

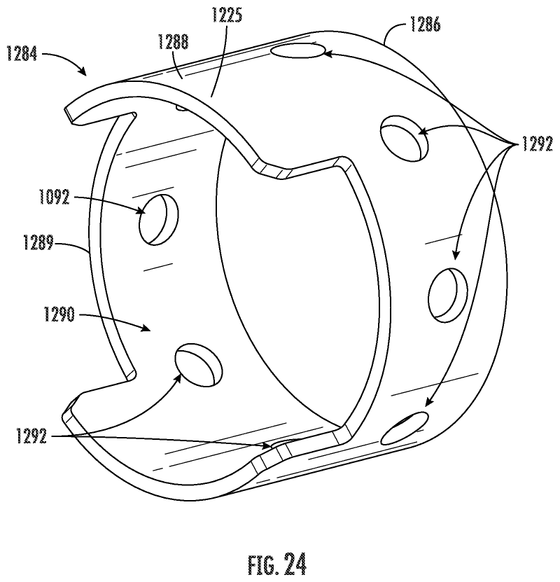

FIGS. 23A and 23B show another implementation of a delivery catheter 1200 that similar to the delivery catheter 1100 but further includes a ring 1284 provided at the proximal end 1222 of the hypotube 1218, like element numbers will be used to identify like elements. The ring 1284 provides extra support for the hypotube 1218 and the distal tip portion 1205 of the delivery catheter 1200. The ring 1284 as shown in FIG. 24 includes a proximal end 1286 and a distal end 1288 with a central lumen 1290 extending longitudinally therethrough. The distal end 1288 of the ring 1284 further includes two axially extending recesses 1289 and two axially extending protrusions 1225, equally spaced around the distal end 1288 of the ring 1284. As illustrated in FIGS. 23A and 23B, the axially extending recesses 1289 of the ring 1284 align with the correspondingly shaped axially extending recesses 1219 of the shoulder 1217 of the outer jacket 1208. Similarly, the axially extending protrusions 1225 of the ring 1284 align with the correspondingly shaped projection of the shoulder 1217 of the outer jacket 1208. When assembled, the axially extending protrusions 1225 of the ring 1284 align with the correspondingly shaped axially extending recess of the hypotube 1218, and the axially extending recesses 1289 of the ring 1284 aligns with the corresponding shaped protrusion 1121 of the hypotube 1218.

In other examples, the distal end of the ring 1284 includes one axially extending recess, no axially extending recesses, or more than two axially extending recesses. Also, in other examples the axially extending recesses are unequally spaced around the distal end of the ring 1284.

In the example shown in FIGS. 23A and 23B, the ring 1284 is fully embedded in and reflowed with the first outer jacket portion 1215 such that the axially extending recesses 1219 of the shoulder 1217 of the outer jacket 1208 and the axially extending recesses 1289 of the ring 1284 are aligned. As described above the and illustrated in FIGS. 23A and 23B, when embedded, distal end 1288 of the ring 1284 is adjacent the shoulder 1217 between the first outer jacket portion 1215 and second outer jacket portion 1216 and, thus, the proximal end 1222 of the hypotube 1218. The ring 1284 further includes eight openings 1292 equally spaced around the ring 1284 such that reflowed material can extend through the eight openings 1292 to facilitate the ring 1284 being reflowed and embedded within the outer jacket 1208. In other examples, the ring 1284 includes more or less than eight openings 1292. In further examples, the ring 1284 does not include any openings.

In other examples, the ring 1284 is only partially embedded in the first outer jacket portion 1215 and/or entirely disposed within the cavity of the outer jacket 1208 adjacent the proximal end of the hypotube 1218. For example, similar to the ring 1084 depicted in FIG. 19, the ring 1284 includes a circumferential extrusion extending from the proximal end of the ring 1284. The circumferential extrusion has a larger inner diameter than the inner diameter of the ring 1284. The modified ring 1284 is at least partially disposed or fully disposed within the first outer jacket portion. In other examples, the first outer jacket portion includes a slot for receiving the ring and/or the circumferential extrusion of the ring.

The ring 1284 shown in FIGS. 23A, 23B and 24 is made of stainless steel, but in other implementations, the ring is composed of at least one of a polymer, metal, or composite material.

Any of the aforementioned examples can further include a pullwire. The pullwire can be coupled to the distal end of the outer jacket such that the delivery catheter is activated when a proximal force is applied to the pullwire. As described in more detail below, a pullwire can be used to provide for active, controlled, flexing of the delivery catheter. For example, it may be desirable to have specific portions of the delivery catheter bend/flex to held reach or accommodate patient anatomy. Additionally, a pull wire can be used to increase the tensile load capacity of the delivery catheter. For example, it may be desirable to increase tensile load on the sheath during prosthetic device delivery, e.g., during valve deployment/removal of the valve from a delivery capsule coupled to the distal end of the sheath. By increasing the tensile load the stability of the sheath is improved, preventing undesirable movement of the sheath and damage to the patient tissue.

By not engaging the pull wire (i.e., providing tensile load to the pullwire) until needed, the catheter can bend omnidirectionally allowing tracking in the patient's anatomy on all planes. Omnidirectional bending is helpful in positioning the prosthetic device at the desired location within the patient. For example, when positioning a prosthetic device such as a prosthetic heart valve, an omnidirectional catheter and heart valve can be rotated into position, ensuring the commissures of the native valve line up with the replacement valve.

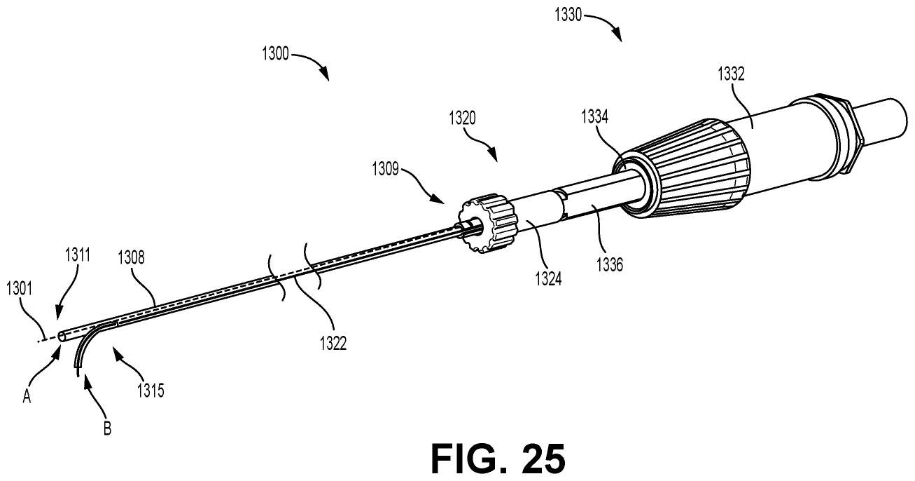

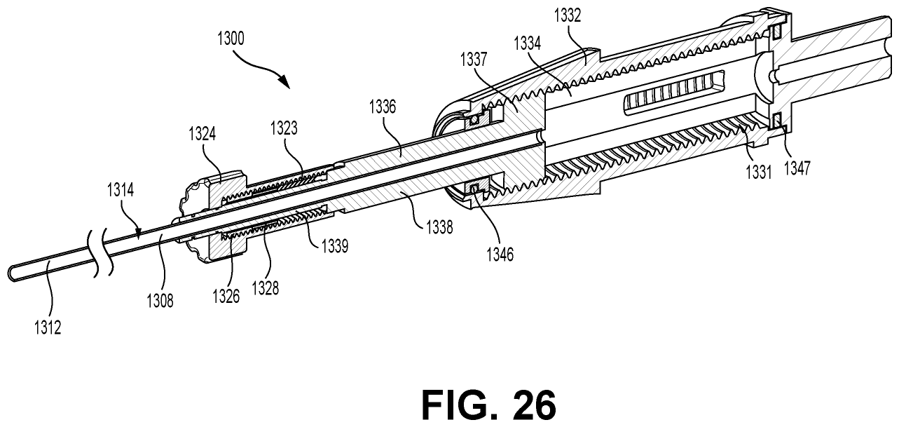

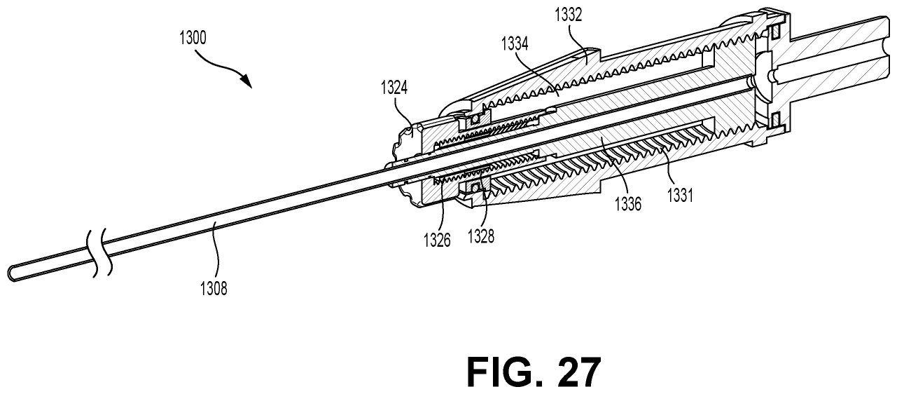

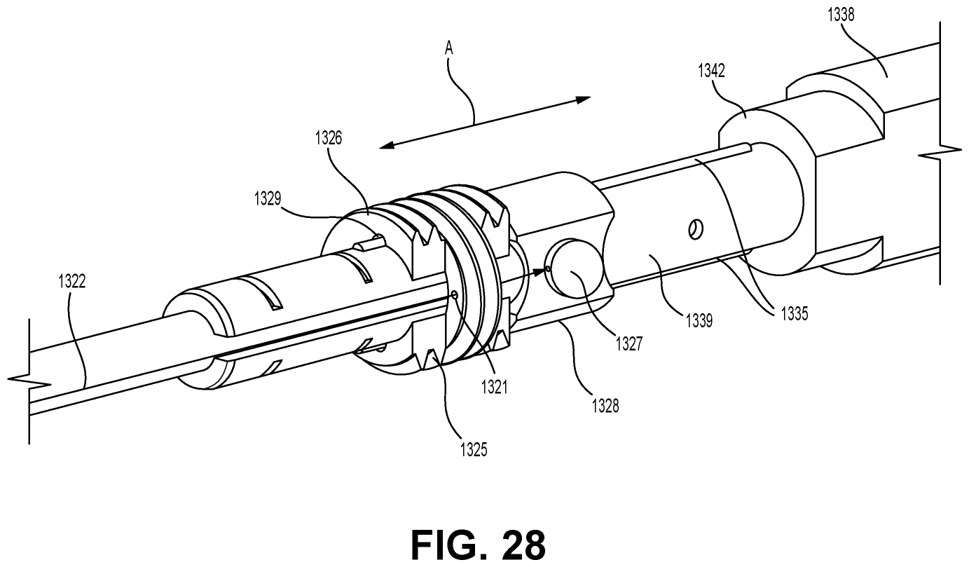

FIG. 25 provides a perspective view of an example flexible delivery catheter 1300 including a valve deployment assembly 1330 and pullwire mechanism 1320. FIG. 26 provides a perspective cross section view of the example flexible delivery catheter with the valve deployment assembly and the pullwire mechanism is a distal position, conversely FIG. 27 provides a perspective cross section view of the example flexible delivery catheter with the valve deployment assembly and the pullwire mechanism is a proximal position. FIG. 28 provides a partial perspective view of the delivery catheter with the knob removed.

The delivery catheter 1300 is similar to the other example delivery catheters described herein and includes similar structure. Differences will be discussed in more detail below, like element numbers will be used to identify like elements.

The delivery catheter 1300 includes an outer jacket 1308, a pullwire mechanism 1320 and a valve deployment assembly 1330. The pullwire mechanism 1320 is coupled to the outer jacket 1308 and directs bending motion of flexible portion(s) of the outer jacket 1308. The valve deployment assembly 1330 is coupled to the outer jacket 1308 and directs proximal and distal movement of the outer jacket 1308. As will be described in more detail below, when a delivery capsule is coupled to the distal end of the delivery catheter 1300, activation of the valve deployment assembly 1330 removes the delivery capsule from the prosthetic device allowing for delivery and implantation into the patient anatomy.

Similar to outer jackets described above, outer jacket 1308 includes a proximal end 1309, a distal end 1311, an inner surface 1312 and an outer surface 1313, and defining a central lumen 1314 extending longitudinally between the proximal end 1309 and the distal end 1311. The outer jacket 1308 includes a flexible portion 1315 extending along a length of the outer jacket 1308 proximal the distal end 1311. FIG. 25 illustrates the outer jacket 1308 in both a straight/unbent configuration (A) and in a curved/bent configuration (B) (without the delivery capsule attached). As described above, the delivery catheter 1300 can include a coil layer (e.g., coil winding extends helically about a longitudinal axis) and/or a hypotube (e.g., tubular structure including plurality of cut-outs) configured to bend omnidirectionally. The coil layer and/or hypotube resist axial compression and tension applied to the delivery catheter 1300, while also facilitating bending of the delivery catheter 1300 in a direction away from the longitudinal axis 1301. As provided in the example delivery catheters described herein, the coil layer and/or a hypotube are positioned adjacent the distal end of the delivery catheter to provide flexibility/bending at the distal end of the delivery catheter to accommodate patient anatomy. The flexible portion 1315 of the outer jacket 1308 is positioned at a location corresponding to the location of the coil layer and/or hypotube, depending on which support structure is within the central lumen 1314 of the outer jacket 1308.

The pullwire mechanism 1320 includes a pullwire 1322 (e.g., a stainless-steel wire) that extends along the outer jacket 1308 and is coupled to the outer jacket 1308 at a location proximate the distal end 1311 of the outer jacket 1308. It is contemplated that the pullwire 1322 can be coupled to other layers structure of the delivery catheter 1300 proximate the distal end, or other appropriate coupling point. The pullwire 1322 can be disposed within or reflowed with the outer jacket 1308. FIG. 25 provides a cutaway portion of the outer jacket 1308 illustrating the pullwire 1322 extending along the length of the outer jacket 1308. For example, the pullwire 1322 can extend along all or a portion of the entire length of the outer jacket 1308/delivery catheter 1300.

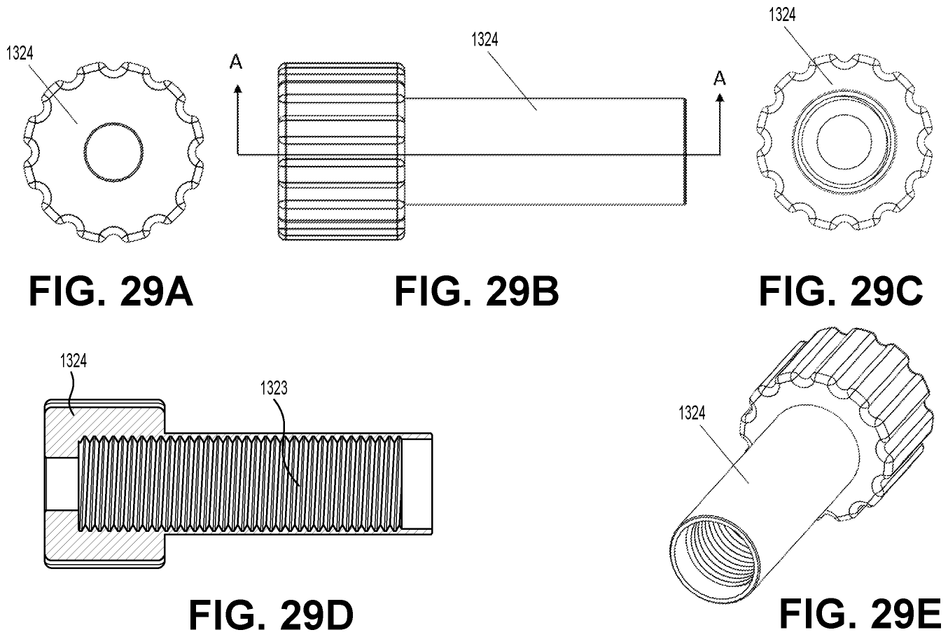

The pullwire mechanism 1320 further includes a knob 1324 coupled to the pullwire 1322. Various views of the example knob of FIG. 25 are illustrated in FIGS. 29A-29E. As described herein, rotational movement of the knob 1324 engages the pullwire 1322 increasing and decreasing the tensile load on the pullwire 1233, and causing the pullwire 1322 to move in a proximal or distal direction. For example, movement of the pullwire 1322 is a proximal direction results in a corresponding bending motion of the flexible portion 1315 of the outer jacket 1308, i.e., movement of the outer jacket 1308 from a straight/unbent configuration (A) to a curved/bent configuration (B). As illustrated in FIG. 25, the bending motion of the distal end of the outer jacket 1308 is in a direction transverse to the longitudinal axis 1301 of the delivery catheter 1300. That is, movement of the pullwire 1322 in the proximal direction, causes the distal end 1311 (flexible portion 1315) of the outer jacket 1308 to curve in a direction transverse to the longitudinal axis 1301 of the delivery catheter 1300. Likewise, movement of the pullwire 1322 in the distal direction (and/or release of the tensile load applied to the pullwire 1322), causes the outer jacket 1308 (e.g., flexible portion 1315) to uncurve/straighten or otherwise bend back toward the longitudinal axis 1301 of the delivery catheter 1300. As described above, activation of the pullwire 1322 (e.g., movement of the pullwire 1322 in the proximal direction), increases a tensile load on the pullwire 1322 and a corresponding increase in tensile load on the outer jacket 1308. When the pullwire 1322 is not engaged (i.e., there is no tension is provided on the pullwire 1322), the flexible portion 1315 of the outer jacket 1308 can bend omnidirectionally any direction away from the longitudinal axis 1301 of the delivery catheter 1300. In some examples, the pullwire 1322 is not engaged during advancement and positioning of the delivery catheter 1300 within the patient. However, the pullwire 1322 is engaged when increased tensile on the delivery catheter 1300 load is needed, for example, during valve deployment/removal of the valve from the delivery capsule 1350.

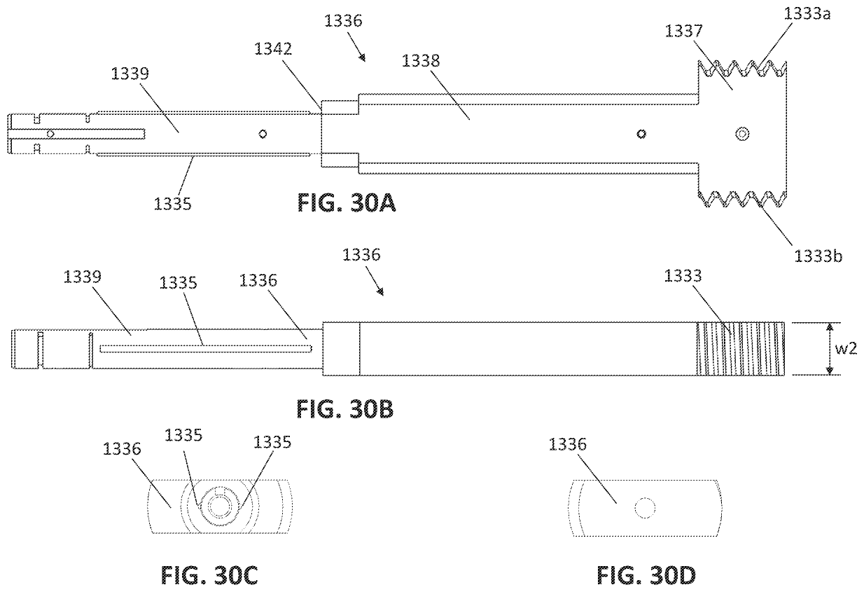

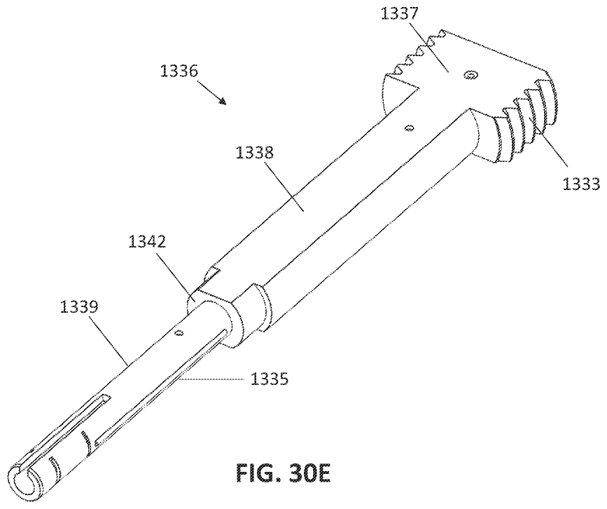

As will be described in more detail below, the valve deployment assembly 1330 includes a coupler 1336 for coupling the pullwire mechanism 1320 to the valve deployment assembly 1330. As illustrated in FIGS. 25 and 26, the knob 1324 is coupled to the coupler 1336 of the valve deployment assembly 1330. The knob 1324 is rotatably coupled to the coupler 1336 such that the knob 1324 rotates with respect to the coupler 1336. As provided in the cross-section view in FIG. 26 and the various views of the example coupler 1336 provided in FIGS. 30A-30E, the coupler 1336 includes the proximal end portion 1337 received within the housing 1332, a main body portion 1338 extending through and beyond a distal opening in the inner member 1334 and a distal opening in the housing 1332, and a distal end portion 1339 extending distally from the main body portion 1338.

The distal end portion 1339 of the coupler 1336 extends within the central lumen of the knob 1324. As provided in FIG. 26, the knob 1324 is rotatably coupled to the coupler 1336 at the distal end portion 1339 such that the proximal end of the knob 1324 is mounted on/coupled to a reduced diameter portion of the distal end portion 1339 of the coupler 1336. A shoulder provided on the inner surface on the knob 1324 abuts a corresponding shoulder 1342 provided on the coupler 1336 such that axial movement of the knob 1324 with respect to the coupler 1336 is prevented (i.e., in the direction identified by arrow A, generally parallel to the longitudinal axis 1301 of the delivery catheter 1300).

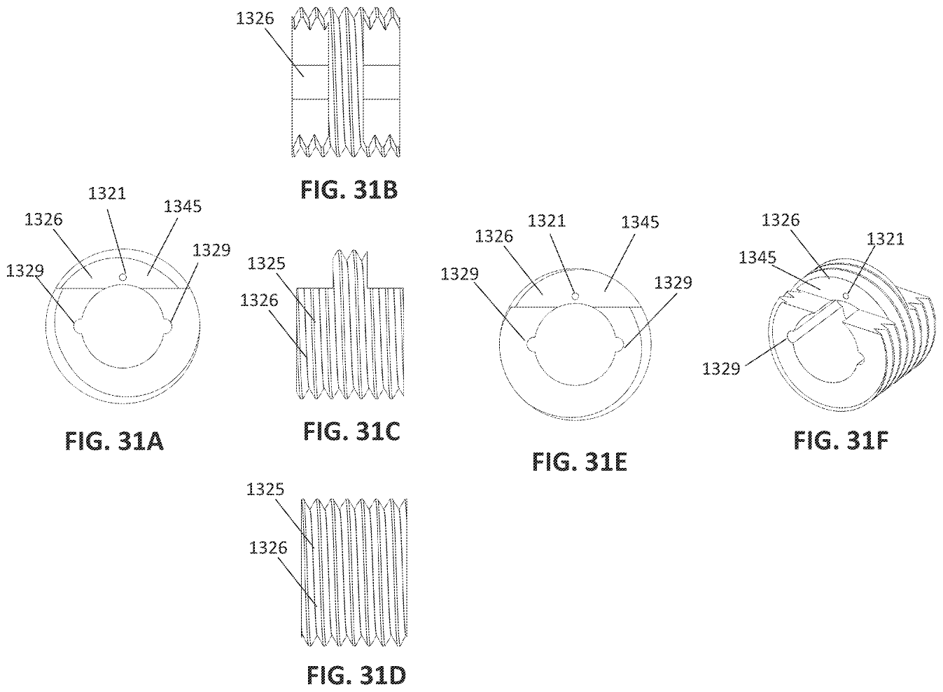

The pullwire mechanism 1320 further includes a threaded sleeve 1326 operatively coupling the knob 1324 and the coupler 1336. Illustrated in FIG. 28 and the various views of the example threaded sleeve 1326 provided in FIGS. 31A-31F, the threaded sleeve 1326 includes a central lumen extending longitudinally therethrough and a threaded outer surface 1325. The threaded sleeve 1326 is positioned on the distal end portion 1339 of the coupler 1336 such that at least a portion of the distal end portion 1339 extends through the central lumen of the threaded sleeve 1326. The threaded sleeve 1326 is axially movably along the coupler 1336, e.g., in the direction along arrow A. The threaded outer surface 1325 of the threaded sleeve 1326 engages a threaded inner surface 1323 of the knob 1324 such that rotational movement of the knob 1324 causes the threaded outer surface 1325 to engage the threaded inner surface 1323 of the knob 1324, resulting in a corresponding axial movement of the threaded sleeve 1326 along the coupler 1336.

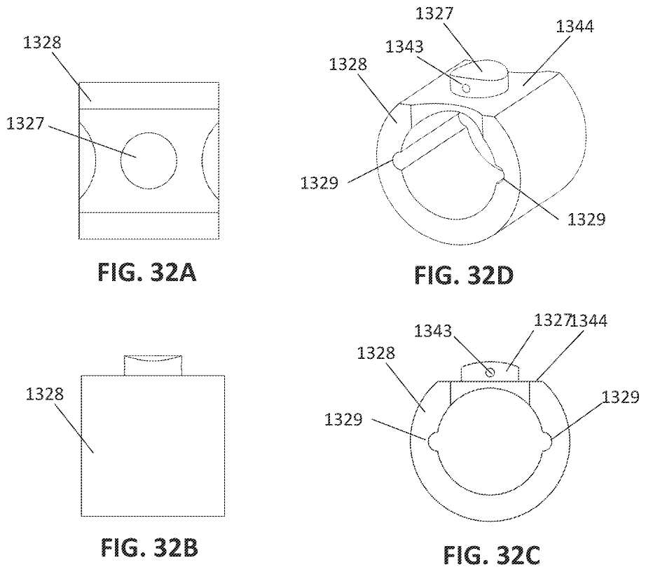

In an example delivery catheter 1300, the pullwire 1322 coupled (e.g., fixedly or removably coupled) to an anchor sleeve 1328. As illustrated in FIG. 28 and the various views of the example anchor sleeve 1328 provided in FIGS. 32A-32D, the pullwire mechanism 1320 includes an anchor sleeve 1328 positioned on and axially movable along the distal end portion 1339 of the coupler 1336 between the threaded sleeve 1326 and the main body portion 1338 of the coupler 1336. As such, rotational movement of the knob 1324 causes the threaded sleeve 1326 to engage the threaded inner surface 1323 of the knob 1324 resulting in a corresponding axial movement of the threaded sleeve 1326 and the anchor sleeve 1328 along the coupler 1336. This results in a corresponding movement axial movement of the pullwire 1322 in the proximal or distal direction (e.g., in the direction identified by arrow A).

The anchor sleeve 1328 includes a central lumen extending therethrough sized and configured to receive the distal end portion 1339 of the coupler 1336. The pullwire 1322 is fixedly coupled to the anchor sleeve 1328 by a mechanical fastener, a chemical fastener, or combinations thereof. For example, the pullwire 1322 is coupled to the anchor sleeve 1328, e.g., within an opening 1343 provided in the anchor sleeve 1328, by a press fit, an interference fit, a snap fit, a mechanical fastener, a weld, and an adhesive, or a combination thereof. As illustrated in FIGS. 28 and 32A-32D, the anchor sleeve 1328 includes an anchor post 1327 extending from an outer surface of the anchor sleeve 1328, and the pullwire 1322 is fixedly coupled to the anchor post 1327 (e.g., mechanical and/or a chemical fastener). The anchor post 1327 is provided on a flat surface 1344 extending parallel to the longitudinal axis 1301 of the catheter.

Coupled to the anchor sleeve 1328, the pullwire 1322 extends distally through/along the threaded sleeve 1326 to the distal end of the outer jacket 1308. As provided in FIGS. 28 and 31A-31F, the pullwire 1322 extends through a longitudinally extending opening 1321 in the threaded sleeve 1326 to the anchor sleeve 1328. The opening 1321 is provided on a flat surface 1345 extending transverse and generally perpendicular to the longitudinal axis 1301 of the catheter. As provided in in FIGS. 31A-31F, the threaded sleeve 1326 includes two flat surfaces 1345, a proximal facing flat surface and a distal facing flat surface, the opening 1321 extending longitudinally therebetween. The opening 1321 extends generally parallel to the longitudinal axis 1301 of the catheter.

In an example delivery catheter 1300, the coupler 1336 and the threaded sleeve 1326 and/or anchor sleeve 1328 include alignment features for fixing the circumferential position of the threaded sleeve 1326 and/or anchor sleeve 1328 with respect to the coupler 1336. For example, as provided in FIGS. 28 and 30A-30E, the coupler 1336 includes an alignment feature 1335 extending longitudinally along an outer surface of the distal end portion 1339. The alignment feature 1335 engages a corresponding alignment feature 1329 provided on an inner surface of at least one of the threaded sleeve 1326 and the anchor sleeve 138. Engagement between the coupler alignment feature 1335 and at least one of the threaded sleeve alignment feature 1329 and the anchor sleeve alignment feature 1329 fixes the circumferential position of the threaded sleeve 1326 and/or anchor sleeve 1328 with respect to the coupler 1336. As provided in FIGS. 28 and 30A-32D, the coupler alignment feature 1335 includes a longitudinally extending ridge projecting from the outer surface of the coupler 1336 and the threaded sleeve alignment feature 1329 and/or the anchor sleeve alignment feature 1329 includes a correspondingly shaped recess provided on an inner surface of the threaded sleeve and/or anchor sleeve. In a further example, the coupler alignment feature 1335 includes a longitudinally extending groove recessed in the outer surface of the coupler 1336 and the threaded sleeve alignment feature 1329 and/or the anchor sleeve alignment feature 1329 includes a correspondingly shaped ridge projecting from an inner surface of the threaded sleeve 1326 and/or anchor sleeve 1328.

As described above, the valve deployment assembly 1330 is coupled to the outer jacket 1308 and directs proximal and distal movement of the outer jacket 1308. By moving the outer jacket 1308 proximally, a delivery capsule (storing a prosthetic device) coupled to the distal end of the delivery catheter 1300/outer jacket 1308 is withdrawn from the prosthetic device allowing delivery and implantation into the patient anatomy.

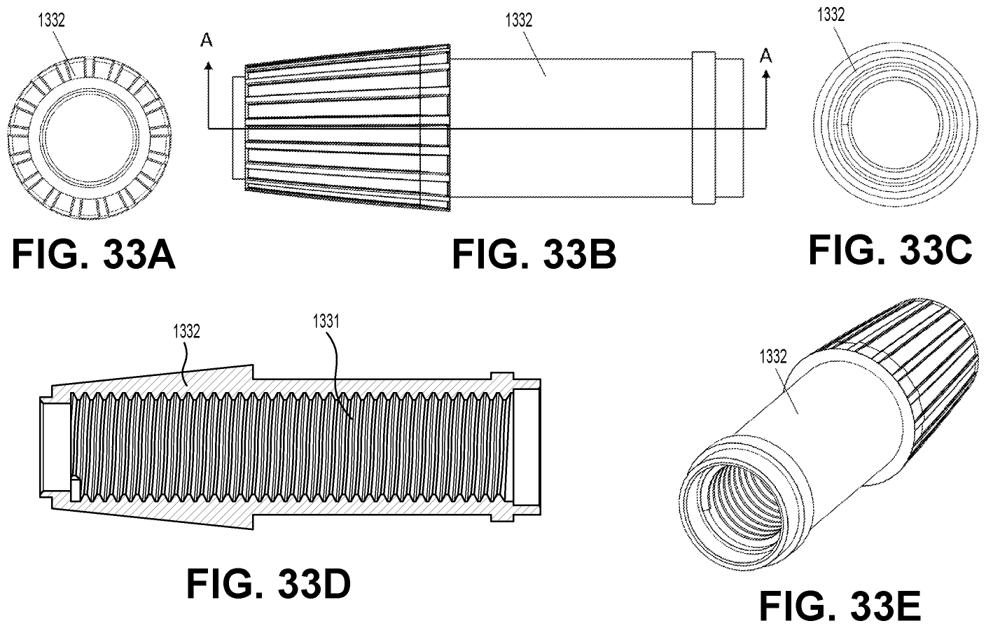

The valve deployment assembly 1330 includes a housing 1332, an inner member 1334, and the coupler 1336 (described above). The housing 1332, as illustrated in the various views of the example housing 1332 provided in FIGS. 33A-33E, includes a central lumen extending therethrough and a textured outer surface configured to for griping by the user. The central lumen of the housing 1332 includes a threaded inner surface 1331. As illustrated in FIG. 26, the inner member 1334 extends within the central lumen of the housing 1332 and is rotatably coupled to the housing 1332.

The coupler 1336 is fixedly coupled to the outer jacket 1308 such that rotational and axial movement of the coupler 1336 results in a corresponding rotational and axial movement of the outer jacket 1308. The coupler 1336 is rotatably coupled to the housing 1332. As illustrated in FIG. 26, a portion of the coupler 1336 (e.g., proximal end portion 1337) extends within the central lumen of the housing 1332 and within the central lumen of the inner member 1334. The proximal end portion 1337 of the coupler 1336 includes a threaded outer surface 1333 that threadingly engages the threaded inner surface 1331 of the housing 1332 such that rotational movement of the housing 1332 causes the threaded inner surface 1331 of the housing 1332 to engage the threaded outer surface 1333 of the coupler 1336, resulting in a corresponding axial movement of the coupler 1336 within the housing 1332, and further resulting in a corresponding axial movement of the outer jacket 1308 in the proximal or distal direction. As described above, the coupler 1336 and outer jacket 1308 are movable with respect to the housing 1332 between an undeployed configuration as shown in FIG. 26, where the coupler 1336 is provided in a distal position with regard to the housing 1332, to a deployed configuration as shown in FIG. 27, where the coupler 1336 and the outer jacket 1308 are located in a proximal position adjacent the proximal end of the inner member 1334/housing 1332. Because the pullwire mechanism 1320 is operably coupled to the valve deployment assembly 1330, movement of the valve deployment assembly 1330/coupler 1336 in the proximal and distal direction results in a corresponding movement of the pullwire mechanism 1320 in the proximal and distal direction.

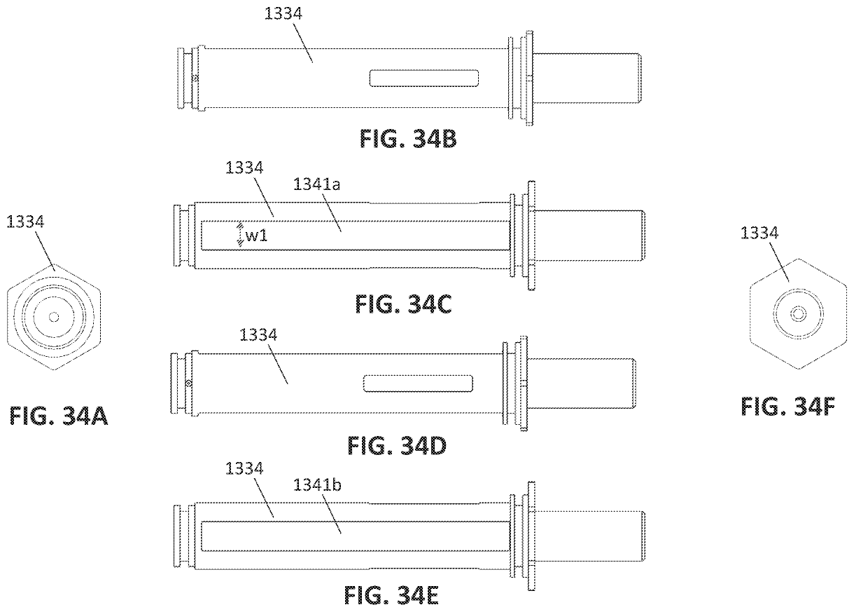

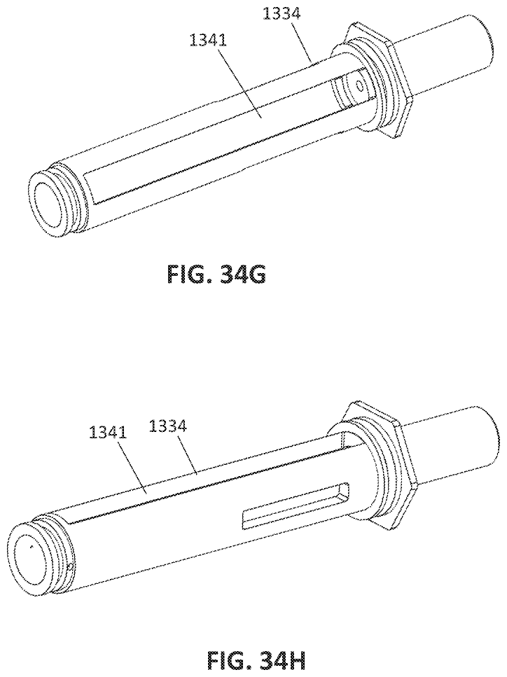

The valve deployment assembly 1330 further includes the inner member 1334. The inner member 1334, as illustrated in the various views of the inner member 1334 provided in FIGS. 34A-34H, includes a longitudinally extending window 1341 extending through a sidewall of the inner member 1334. As illustrated in FIGS. 26 and 27, the proximal end portion 1337 of the coupler 1336 including the threaded outer surface 1333 extends through the window 1341. In some examples, the inner member 1334 includes more than one longitudinally extending window 1341 extending through the sidewall of the inner member 1334. As illustrated in FIGS. 34A-34H, the inner member 1334 includes two larger longitudinally extending windows 1341 extending through the cylindrically-shaped sidewall of the inner member 1334 and two smaller longitudinally extending windows spaced circumferentially between the windows 1341. As provided in FIGS. 26 and 27, the proximal end portion 1337 of the proximal end of the coupler 1336 defines a generally dogbone or T-shape including the two opposing threaded outer surfaces 1333a, 1333b. When assembled, and as illustrated in FIGS. 26-27, each of the opposing threaded outer surfaces 1333a, 1333b extend through a corresponding window 1341a, 1341b of the inner member 1334. The opposing threaded outer surfaces 1333a, 1333b each engage the threaded inner surface 1331 of the housing 1332 and drive rotational movement between the coupler 1336 and the housing 1332.

As provided in FIGS. 34C and 34E, the window(s) 1341 extends along a majority of a total length of the inner member 1334. As illustrated in FIGS. 30B and FIGS. 34C and 34E, the circumferential width (w1) of the window 1341 is greater than the (circumferential) width (w2) of the threaded outer surface 1333 of the coupler 1336. It is contemplated that the side wall/edge surface of the proximal end portion 1337/1333 of the coupler 1336 extending into and/or through the window 1341, slidingly engages a corresponding side wall surface of the window 1341.

As illustrated in FIGS. 26 and 27, the housing 1332 is mounted on/coupled to the inner member 1334. For example, the proximal end and the distal end of the housing 1332 is coupled to the inner member 1334. For example, a distal end of the inner member 1334 includes a housing coupling portion including a shoulder for engaging a corresponding shoulder provided on an inner surface of the housing 1332. Engagement between the housing shoulder and the inner member shoulder limits axial movement of the inner member in a distal direction. Similarly, the proximal end of the inner member 1334 includes a proximal housing coupling portion including a shoulder for engaging at least one of a shoulder and a distal end surface of the housing 1332, wherein engagement between the inner member shoulder and at least one of the housing shoulder and the distal end surface of the housing 1332 limits axial movement of the inner member 1334 in a proximal direction.

As illustrated in FIGS. 26 and 27, the inner member 1334 includes an o-ring 1346 provided between the inner member 1334 and the housing 1332 proximate a distal end of the housing 1332. The o-ring 1346 providing a sealing engagement between the housing 1332 and the inner member 1334. Additionally/alternatively, the o-ring 1346 provides a bearing surface between the housing 1332 and inner member 1334 to facilitate rotational movement therebetween. The o-ring 1346 is provided in a recess extending circumferentially around an outer surface of the inner member 1334 proximate a distal end of the inner member 1334. The inner member further includes a second o-ring 1347 provided between the inner member 1334 and the housing 1332 proximate a proximal end of the housing 1332. The second o-ring 1347 is provided in a recess extending circumferentially around an outer surface of the inner member 1334 proximate a proximal end of the inner member 1334.

As provided in FIGS. 36 and 27, the proximal end of the inner member 1334 extends proximally through and beyond a proximal opening in the housing 1332. For example, the cylindrical portion of the inner member 1334 extending proximally from the housing 1332 providing a handle or gripping surface for the user. Additionally/alternatively, the proximal end portion can be used to couple the delivery catheter 1300 to other medical equipment/tools.

While FIGS. 26 and 27 illustrate the pullwire mechanism 1320 distal to the valve deployment assembly 1330, it is contemplated that components can be reoriented along the delivery catheter 1300 such that the valve deployment assembly 1330 is distal to the pullwire mechanism 1320.

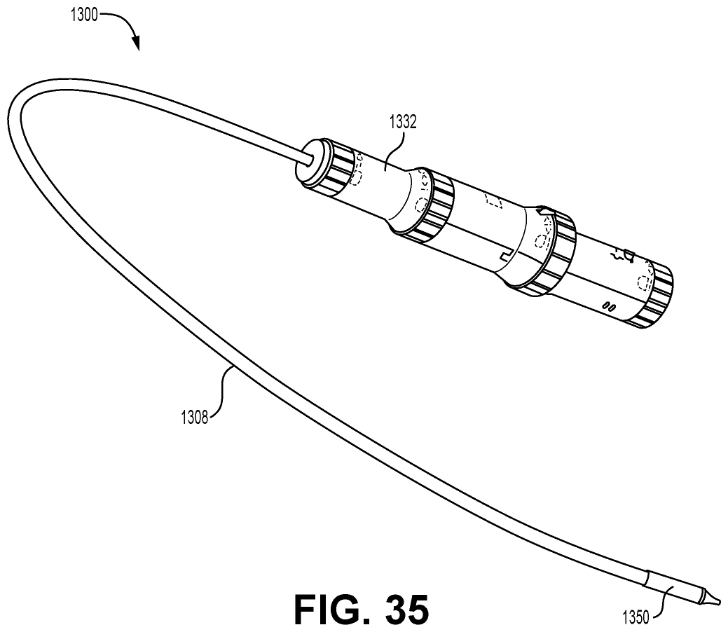

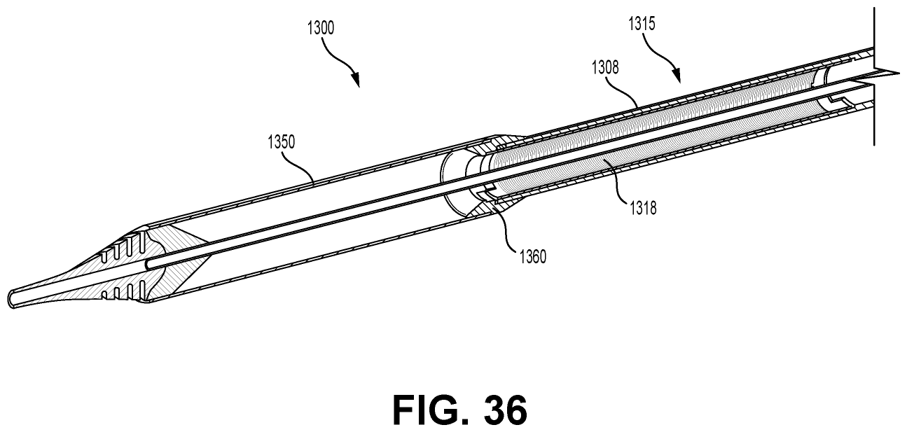

As described above, the delivery catheter 1300 can include a delivery capsule 1350 coupled to the distal end of the outer jacket 1308. FIG. 35 provides a perspective view of an example delivery catheter 1300 including a delivery capsule 1350 coupled to the distal end of the outer jacket 1308. FIG. 36 provides a partial cross section view of the distal end of the delivery catheter and the delivery capsule 1350 of FIG. 35 (including distal nose cone). The central lumen of the delivery capsule 1350 is sized and configured to receive a prosthetic device 1355 (e.g., prosthetic heart valve) in a crimped or non-expanded configuration therein.

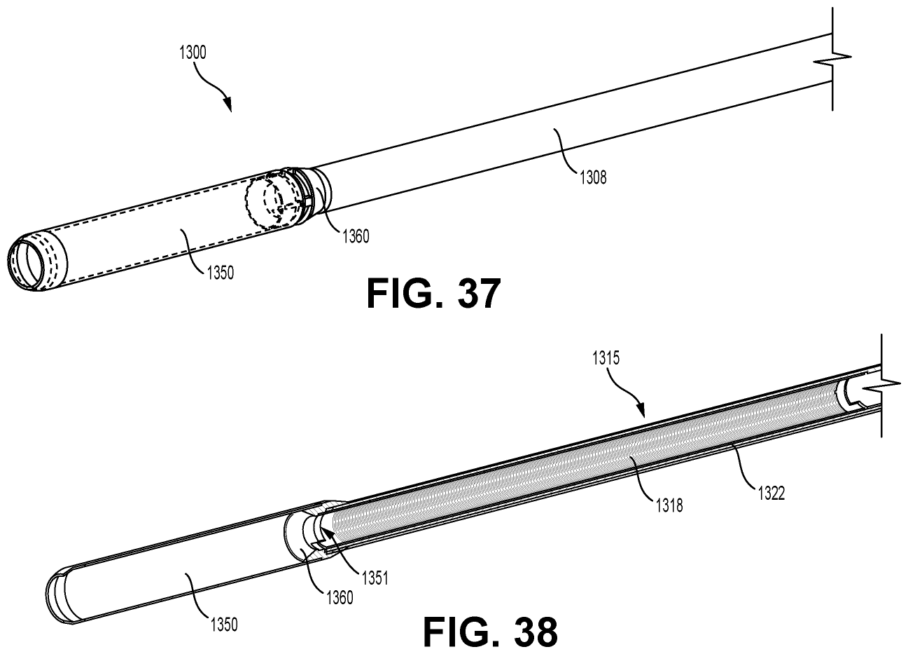

As described above with regard to example delivery catheters, the delivery capsule 1350 is coupled to the outer jacket 1308 via a delivery capsule coupler 1360 similar to those described herein. FIG. 37 provides a perspective view of the delivery catheter 1300, delivery capsule coupler 1360, and delivery capsule 1350 of FIG. 36 (without the nosecone extending through the distal opening of the delivery capsule 1350). FIG. 38 provides a partial perspective cross section view of the catheter of FIG. 37.

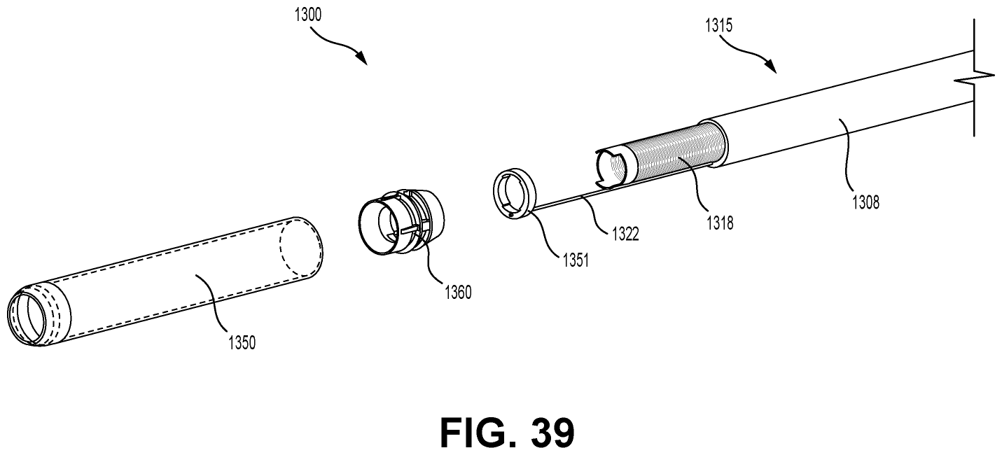

As illustrated in FIG. 38 and the exploded view provided in FIG. 39, the example delivery catheter includes a hypotube 1318. It is contemplated that the delivery catheter can alternatively include a coiled layer as described above. As provided in FIGS. 38 and 39, the flexible portion 1315 of the outer jacket 1308 is provided at an axial location corresponding to the axial location of the hypotube 1318 (and/or coil layer). The example catheter, the pullwire 1322 extends through/along the outer jacket 1308 and is coupled to the distal end of the outer jacket 1308 at a mounting ring 1351 provided between the distal end of the outer jacket 1308 and the delivery capsule 1350. For example, the mounting ring 1351 is provided between the distal end of the outer jacket 1308 and the delivery capsule coupler 1360. In some examples the pullwire 1322 is fixedly coupled to the mounting ring 1351. Alternatively, the pullwire 1322 is removably coupled to the mounting ring 1351.

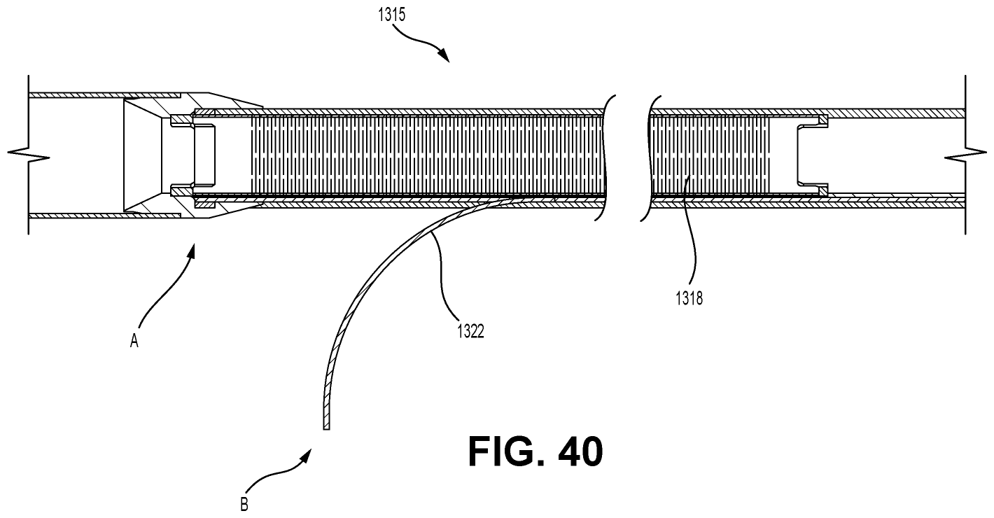

FIG. 40 illustrates bending/curving of the catheter in response to tension applied to the pullwire 1322. As provided in FIG. 40, the outer jacket 1308 bends/curves along the flexible portion 1315 corresponding to the location of the hypotube 1318. For example, movement of the pullwire 1322 is a proximal direction results in a corresponding bending motion of the flexible portion 1315 of the outer jacket 1308, i.e., movement of the outer jacket 1308 from a straight/unbent configuration along line A to a curved/bent configuration along line B. Likewise, movement of the pullwire 1322 in the distal direction or otherwise releasing the tension applied to the pullwire results in the outer jacket 1308/hypotube 1318 returning towards the straight/unbent configuration along line A.

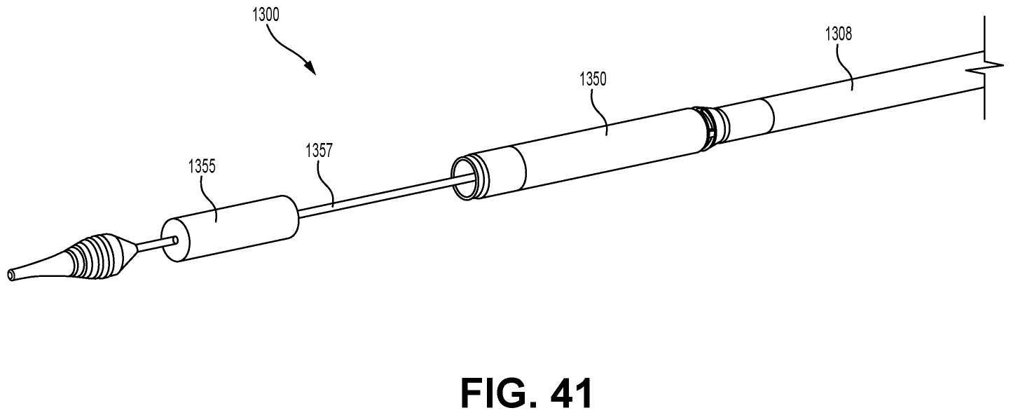

As described above, once the distal end of the delivery catheter 1300 is at the desired position within the patient anatomy, the valve deployment assembly 1330 can be activated to move the outer jacket 1308 (and coupler 1336) in a proximal direction, thereby withdrawing the delivery capsule 1350, coupled to the distal end of the outer jacket, from the prosthetic device. Specifically, rotational movement of the housing 1332 causes the threaded inner surface 1331 of the housing 1332 to engage the threaded outer surface 1333 of the coupler 1336. This results in a corresponding axial movement of the coupler 1336 within the housing 1332. Axial movement of the coupler 1336 in the proximal direction results in a corresponding axial movement of the outer jacket 1308 and delivery capsule 1350 in the proximal direction and removes/withdraws the delivery capsule 1350 from a prosthetic device 1355 stored within the delivery capsule 1350, as illustrated in FIG. 41. FIG. 41 provides a schematic representation of a prosthetic device 1355 coupled to a device catheter 1357 extending through a central lumen of the outer jacket 1308. The device catheter 1357 is movable within the central lumen of the outer jacket 1308 to position the prosthetic device 1355 at a desired position with the patient. During axial movement of the outer jacket 1308 in the proximal direction, the device catheter 1357 maintains the axial location of the prosthetic device 1355 such that the delivery capsule 1350 moves in a proximal direction, removing/withdrawing the delivery capsule 1350 from the prosthetic device 1355. To assist in removing the delivery capsule 1350 from prosthetic device 1355, the pullwire mechanism 1320 is engaged and the pullwire 1322 is moved in a proximal direction during axial movement of the outer jacket 1308 in the proximal direction. That is, the pullwire mechanism 13020 is engaged when tensile loads are needed such as during prosthetic device deployment, this increases the tensile load on the outer jacket 1308 providing stability to the outer jacket 1308.

### Methods

Also disclosed herein is a method of making the flexible delivery catheter 700 including a braided layer 726 with a variable braid pattern. The method includes forming an outer jacket 708 and a braided layer 726 as described above and shown in FIG. 7. The braided layer is disposed within the inner lumen of the outer jacket 708. The braided layer 726 and the outer jacket 708 are coupled together and reflowed together to form a single catheter. The braided layer 726 is weaved together to form a pattern of varying density and pitch along the axial length of the hypotube.

In examples wherein the outer jacket 708 comprises a plurality of longitudinal segments as described above, each of the plurality of longitudinal segments are reflowed together with at least one an adjacent longitudinal segment having a differing elastic property. In examples that include tensile stiffening fibers 827, the tensile stiffening fibers 827 are coupled to the braided layer 826 by weaving the tensile stiffening fiber 827 at least partially into braided pattern of the braided layer 826. The tensile stiffening fiber 827 is inserted into the distal tip of the outer jacket 808 and securing it thereto (e.g., via reflow process).

Also disclosed herein is a method of making a flexible delivery catheter 800. The method includes forming an outer jacket 808 at least one tensile stiffening fiber 827. The at least one tensile stiffening fiber 827 is embedded into the outer layer and coupled thereto.

In examples wherein the outer jacket 808 comprises a plurality of longitudinal segments as described above, each of the plurality of longitudinal segments are reflowed together with at least one an adjacent longitudinal segment having a differing elastic property. The at least one tensile stiffening fiber 827 is inserted into the distal tip of the outer jacket 808 and securing it thereto.

Also disclosed herein is a method of making a flexible delivery catheter 900, 1100. The method includes forming an outer jacket that includes a first portion with a first inner diameter and a second portion with a second inner diameter, forming a coil layer or a hypotube, providing a coupler, disposing the coil layer or the hypotube within the central lumen of the outer jacket such that the hypotube abuts the inner surface of the second portion of the outer jacket, and coupling the coupler to the distal end of the outer jacket such that the hypotube is secured within the second portion of the outer jacket.

A method of deploying a prosthetic into a patient is also disclosed herein. The method includes using a delivery system. The delivery system includes a flexible delivery catheter as described above 300, 600, 700, 800, 900, 1000, 1100, 1200, or 1300. The catheter is advanced into a vessel of a patient to be utilized during a medical procedure such as insertion of a prosthetic heart valve. The prosthetic is then advanced into the patient through the catheter and the catheter is removed.

It will be appreciated that examples of the heart valve delivery system provide improved devices and methods for advancing a prosthetic heart valve through a patient's vasculature. In one example, the cooperation of components described herein allows a prosthetic valve to be advanced through the patient's vasculature and around the aortic arch in a safe manner. Accordingly, the delivery system enables advancement of a prosthetic valve around the aortic arch without requiring the introduction of an outer sheath into the aortic arch. This is an advantageous feature because the use of a sheath would increase the diameter of the delivery system, thereby complicating the delivery of the valve. In addition to providing an improved steering mechanism for navigating the aortic arch without damaging the inner wall of the aorta, it will be appreciated by those skilled in the art that the delivery system provides excellent pushability such that the physician has excellent control over the movement and location of the prosthetic valve during advancement into the native valve. This feature is particularly advantageous when traversing stenotic valve leaflets. Accordingly, examples of the present invention provide an improved delivery system for advancing a prosthetic valve to the site of a native aortic valve using a steerable assembly that eliminates the need for an outer sheath in the aorta, while providing sufficiently pushability to pass through narrow vasculature and/or stenotic valve leaflets. As a result, examples of the present invention provide improved devices and methods for percutaneously advancing a balloon-expandable prosthetic valve to the site of a stenotic aortic valve using a retrograde approach.

### EXAMPLE ASPECTS

In view of the described processes and compositions, hereinbelow are described certain more particularly described aspects of the disclosures. These particularly recited aspects should not, however, be interpreted to have any limiting effect on any different claims containing different or more general teachings described herein, or that the “particular” aspects are somehow limited in some way other than the inherent meanings of the language and formulas literally used therein.

Example 1 A flexible delivery catheter comprising: an outer jacket, having a proximal end, a distal end, an inner surface and an outer surface, and defining a central lumen extending longitudinally between the proximal end and the distal end; a coil layer disposed at least partially within the central lumen of the outer jacket, the coil layer comprising a coil winding and having a proximal end, a distal end, an inner surface, and an outer surface, wherein the coil winding extends helically about a longitudinal axis and defines a central lumen between the proximal end and the distal end; and a coupler provided at a distal end of the outer jacket, the coupler having a proximal end, a distal end, and outer surface and defining a central lumen extending longitudinally between the proximal end and the distal end, wherein the outer jacket includes a first portion with a first inner diameter and a second portion with a second inner diameter, the first portion adjacent the proximal end of the outer jacket, the second portion adjacent the distal end of the outer jacket, the second inner diameter being greater than the first inner diameter, wherein the outer jacket further includes a shoulder disposed between the first and second portions, and wherein the coupler and the second portion of the outer jacket form a cavity, and the coil layer is disposed within the cavity such that the coil layer abuts the inner surface of the second portion of outer jacket.

Example 2: The delivery catheter according to any example herein, particularly example 1, wherein the delivery catheter is omnidirectionally flexible and configured to bend in any direction away from the longitudinal axis of the delivery catheter.

Example 3: The delivery catheter according to any example herein, particularly examples 1-2, wherein the delivery catheter includes a distal tip portion extending from the distal end of the delivery catheter along a length of the delivery catheter and an elongated body portion extending from the proximal end of the delivery catheter to the distal tip portion of the delivery catheter.

Example 4: The delivery catheter according to any example herein, particularly example 3, wherein the distal tip portion corresponds to the axial location of the cavity.

Example 5: The delivery catheter according to any example herein, particularly examples 3-4, wherein an outer diameter of the distal tip portion corresponds with an outer diameter of the elongated body portion.

Example 6: The delivery catheter according to any example herein, particularly examples 3-4, wherein an outer diameter of the distal tip portion is greater than an outer diameter of the elongated body portion.

Example 7: The delivery catheter according to any example herein, particularly examples 5 or 6, wherein the outer diameter of the elongated body portion tapers radially outwardly to the outer diameter of the distal tip portion.

Example 8: The delivery catheter according to any example herein, particularly examples 3-7, wherein an outer diameter of the outer jacket, including the distal tip portion and the elongated body portion, ranges between about 4.5 mm and about 6 mm.

Example 9: The delivery catheter according to any example herein, particularly examples 3-8, wherein an outer diameter of the outer jacket, including the distal tip portion and the elongated body portion ranges between about 5 mm and about 6 mm.

Example 10: The delivery catheter according to any example herein, particularly examples 3-9, wherein an outer diameter of the outer jacket, including the distal tip portion and the elongated body portion is about 5.4 mm (for example, 0.213 inches).

Example 11: The delivery catheter according to any example herein, particularly examples 1-10, wherein the first inner diameter of the outer jacket and/or the coil layer ranges between about 3.5 mm and about 5 mm.

Example 12: The delivery catheter according to any example herein, particularly examples 1-11, wherein the first inner diameter of the outer jacket and/or the coil layer ranges between about 4.0 mm and about 4.5 mm.

Example 13: The delivery catheter according to any example herein, particularly examples 1-12, wherein the first inner diameter of the outer jacket and/or the coil layer is about 4.4 mm.

Example 14: The delivery catheter according to any example herein, particularly examples 1-13, wherein the second inner diameter of the outer jacket ranges between about 4.6 mm and about 5.4 mm.

Example 15: The delivery catheter according to any example herein, particularly examples 1-14, wherein an outer diameter of the coil layer is less than the second inner diameter of the outer jacket.

Example 16: The delivery catheter according to any example herein, particularly examples 1-15, wherein an outer diameter of the coil layer corresponds with the second inner diameter of the outer jacket.

Example 17: The delivery catheter according to any example herein, particularly examples 1-16, wherein an outer diameter of the coil layer is greater than the first inner diameter of the outer jacket.

Example 18: The delivery catheter according to any example herein, particularly examples 1-17, wherein an outer diameter of the coil layer ranges between about 4.6 mm and about 5.4 mm.

Example 19: The delivery catheter according to any example herein, particularly examples 1-18, wherein a length of the coil layer corresponds to a length of the cavity, wherein the length of the coil layer is measured in a direction along the longitudinal axis of the delivery catheter.

Example 20: The delivery catheter according to any example herein, particularly examples 1-19, wherein a length of the coil layer is less than a length of the cavity.

Example 21: The delivery catheter according to any example herein, particularly examples 1-20, wherein a length of the coil layer corresponds to a length of the distal tip portion.

Example 22: The delivery catheter according to any example herein, particularly examples 1-21, wherein the coil layer has a length less than 305 mm (12 inches), wherein the length of the distal tip portion is measured in a direction along the longitudinal axis of the delivery catheter.

Example 23: The delivery catheter according to any example herein, particularly examples 1-22, wherein the coil layer has a length less than 254 mm (10 inches).

Example 24: The delivery catheter according to any example herein, particularly examples 1-23, wherein the length of the coil layer ranges between 76 mm (3 inches) and 254 mm (10 inches).

Example 25: The delivery catheter according to any example herein, particularly examples 1-24, wherein the length of the coil layer ranges between 178 mm (7 inches) and 254 mm (10 inches).

Example 26. The delivery catheter according to any example herein, particularly examples 1-25, wherein the length of the coil layer is about 215.9 mm (8.5 inches).

Example 27: The delivery catheter according to any example herein, particularly examples 1-26, wherein an overall length of the outer jacket ranges between about 762 mm (30 inches) to about 1178 mm (70 inches).

Example 28: The delivery catheter according to any example herein, particularly examples 1-27, wherein an overall length of the outer jacket ranges between about 889 mm (35 inches) to about 1397 mm (55 inches).

Example 29: The delivery catheter according to any example herein, particularly examples 1-28, wherein an overall length of the outer jacket is about 1320.8 mm (52 inches).

Example 30: The delivery catheter according to any example herein, particularly examples 1-29, further including a delivery capsule with a central lumen, wherein the coupler couples the delivery capsule to the outer jacket.

Example 31: The delivery catheter according to any example herein, particularly example 30, wherein the central lumen of the delivery capsule is sized and configured to receive a prosthetic heart valve in a crimped or non-expanded configuration therein.

Example 32: The delivery catheter according to any example herein, particularly examples 30 or 31, further including a prosthetic heart valve in a crimped or non-expanded configuration within the central lumen of the delivery capsule.

Example 33: The delivery catheter according to any example herein, particularly examples 30-32, wherein an outer diameter of the delivery catheter at the delivery capsule is greater than an outer diameter of the outer jacket.

Example 34: The delivery catheter according to any example herein, particularly examples 30-33, wherein the outer diameter of the delivery capsule ranges between about 7 mm and about 9 mm.

Example 35: The delivery catheter according to any example herein, particularly examples 30-34, wherein the coupler is fixedly coupled to at least one of the outer jacket and/or the delivery capsule.

Example 36: The delivery catheter according to any example herein, particularly examples 30-35, wherein the coupler is coupled to the delivery capsule by at least one of a press fit, an interference fit, a snap fit, a mechanical fastener, a weld, and an adhesive, wherein the coupler is coupled to the outer jacket by at least one of a press fit, an interference fit, a snap fit, a mechanical fastener, a weld, and an adhesive. Example 37: The delivery catheter according to any example herein, particularly examples 30-36, wherein the outer surface of the coupler includes a recess for receiving the delivery capsule such that an outer diameter of the delivery capsule corresponds with an outer diameter of the coupler.

Example 38: The delivery catheter according to any example herein, particularly examples 30-37, wherein the outer surface of the coupler adjacent the outer jacket includes a decreasing taper between a larger outer diameter delivery capsule and a reduced diameter outer jacket.

Example 39: The delivery catheter according to any example herein, particularly examples 30-38, where the inner diameter (for example, central lumen) of the coupler includes a shoulder, wherein the distal end of the coil layer abuts the shoulder of the coupler securing the coil layer between the shoulder of the coupler and the shoulder of the outer jacket.

Example 40: The delivery catheter according to any example herein, particularly example 39, wherein the shoulder of the coupler comprises a radially extending protrusion extending from the central lumen of the coupler.

Example 41: The delivery catheter according to any example herein, particularly examples 39-40, wherein a diameter of the radially extending protrusion corresponds to a diameter of the central lumen of the coil layer.

Example 42: The delivery catheter according to any example herein, particularly examples 30-41, wherein the central lumen of the coupler adjacent the delivery capsule includes an increasing taper between smaller diameter central lumen of the coupler and a larger outer diameter central lumen of the delivery capsule.

Example 43: The delivery catheter according to any example herein, particularly examples 1-42, wherein the central lumen of the coupler includes a cylindrical portion adjacent the proximal end, an increasing taper portion adjacent the distal end, and a radially extending protrusion between the cylindrical portion and the increasing taper portion, wherein a diameter of the radially extending protrusion is less than a diameter of the cylindrical portion.

Example 44: The delivery catheter according to any example herein, particularly examples 1-43, wherein the coil layer is wound to resist axial compression and tension applied to the delivery catheter, and the coil layer facilitates bending of the delivery catheter in a direction away from the longitudinal axis of the delivery catheter.

Example 45: The delivery catheter according to any example herein, particularly examples 1-44, wherein the coil layer is disposed in the cavity such that the proximal end of the coil layer abuts the shoulder of the outer jacket, and the distal end of the coil layer abuts a radially extending protrusion of the coupler.

Example 46: The delivery catheter according to any example herein, particularly examples 1-45, wherein the coil layer includes a maximum gap/spacing of 0.25 mm (0.010 inches) between adjacent turns of the coil winding.

Example 47: The delivery catheter according to any example herein, particularly example 46, wherein an outer surface of adjacent turns of the coiled winding contacts along a circumferential length of the coiled winding.

Example 48: The delivery catheter according to any example herein, particularly examples 1-47, wherein the coil winding has a diameter ranging between about 0.003 inches and about 0.010 inches.

Example 49: The delivery catheter according to any example herein, particularly examples 1-48, wherein the coil winding has a diameter ranging between about 0.004 inches and about 0.008 inches.

Example 50: The delivery catheter according to any example herein, particularly example 49, wherein the coil winding has a diameter of about 0.004 inches.

Example 51: The delivery catheter according to any example herein, particularly example 49, wherein the coil winding has a diameter of about 0.008 inches.

Example 52: The delivery catheter according to any example herein, particularly examples 1-51, wherein the coil winding has a constant pitch along an entire length of the coil winding.

Example 53: The delivery catheter according to any example herein, particularly examples 1-51, wherein the coil winding has a varying pitch along an entire length of the coil winding.

Example 54: The delivery catheter according to any example herein, particularly example 53, wherein a pitch of the coil winding at a proximal end of the distal tip portion is less than a pitch of the coil winding at the distal end of the distal tip portion.

Example 55: The delivery catheter according to any example herein, particularly examples 1-54, wherein the coil winding has between about 55 and 120 turns per inch.

Example 56: The delivery catheter according to any example herein, particularly examples 1-55, wherein the coil winding is composed of at least one of a polymer, a metal, a composite.

Example 57: The delivery catheter according to any example herein, particularly example 56, wherein the coil winding comprises a stainless-steel wire.

Example 58: The delivery catheter according to any example herein, particularly example 57, wherein the coil winding comprises 300 series stainless steel.

Example 59: The delivery catheter according to any example herein, particularly examples 57-58, wherein the coil winding comprises 400 series stainless steel.

Example 60: The delivery catheter according to any example herein, particularly examples 1-59, wherein the coil winding has a rectilinear or curvilinear shape in cross section.

Example 61: The delivery catheter according to any example herein, particularly examples 1-60, further including a braided layer having a proximal end and a distal end and comprising a plurality of fibers disposed in a braided pattern and defining a central lumen having an inner surface and an outer surface.

Example 62: The delivery catheter according to any example herein, particularly example 61, wherein the braided layer is at least partially disposed within the outer jacket.

Example 63: The delivery catheter according to any example herein, particularly examples 61-62, wherein the braided layer extends along an entire length of the outer jacket.

Example 64: The delivery catheter according to any example herein, particularly examples 61-62, wherein the braided layer extends along a portion of an entire length of the outer jacket.

Example 65: The delivery catheter according to any example herein, particularly example 64, wherein the delivery catheter includes a distal tip portion extending from the proximal end of the delivery capsule along a length of the delivery catheter and an elongated body portion extending from the proximal end of the delivery capsule to the distal tip portion of the delivery catheter, wherein the braided layer extends along the elongated body portion.

Example 66: The delivery catheter according to any example herein, particularly example 65, wherein the braided layer extends along at least a portion of the distal tip portion that corresponds to a proximal end of the coil layer.

Example 67: The delivery catheter according to any example herein, particularly examples 65-66, wherein the braided layer extends along at least a portion of the distal tip portion and a corresponding length of the coil layer.

Example 68: The delivery catheter according to any example herein, particularly examples 61-67, wherein the braided layer extends into the first portion of the outer jacket.

Example 69: The delivery catheter according to any example herein, particularly examples 61-68, wherein the braided layer comprises of a plurality of interwoven fibers.

Example 70: The delivery catheter according to any example herein, particularly example 69, wherein each of the plurality of interwoven fibers has a diameter ranging between about 0.002 inches and about 0.006 inches.

Example 71: The delivery catheter according to any example herein, particularly example 70, wherein each of the plurality of interwoven fibers has a diameter ranging between about 0.003 inches and about 0.005 inches.

Example 72: The delivery catheter according to any example herein, particularly example 71, wherein each of the plurality of interwoven fibers has a diameter of about 0.004 inches.

Example 73: The delivery catheter according to any example herein, particularly examples 61-72, wherein the plurality of interwoven fibers comprises about 10 to about 20 individual fibers.

Example 74: The delivery catheter according to any example herein, particularly example 73, wherein the plurality of interwoven fibers comprises 16 individual fibers.

Example 75: The delivery catheter according to any example herein, particularly examples 61-74, wherein the braided layer has a braid density between about 20 to about 30 picks per inch.

Example 76: The delivery catheter according to any example herein, particularly examples 61-75, wherein the braid density varies along the axial length of the catheter.

Example 77: The delivery catheter according to any example herein, particularly example 61-76, wherein the braid density of the braided layer is about 25 picks per inch.

Example 78: The delivery catheter according to any example herein, particularly examples 61-77, wherein the braided layer comprises of a plurality of interwoven fibers, wherein each of the plurality of interwoven fibers have a rectilinear or curvilinear shape in cross section.

Example 79: The delivery catheter according to any example herein, particularly examples 61-78, wherein the braided layer comprises of a plurality of interwoven fibers, wherein the plurality of interwoven fibers are composed of at least one of a polymer, a metal, a composite.

Example 80: The delivery catheter according to any example herein, particularly example 79, wherein the plurality of interwoven fibers includes stainless steel wire.

Example 81: The delivery catheter according to any example herein, particularly example 79, wherein the plurality of interwoven fibers is composed of a liquid crystal polymer.

Example 82: The delivery catheter according to any example herein, particularly example 79, wherein the plurality of interwoven fibers is composed of a Kevlar (e.g., Technora®).

Example 83: The delivery catheter according to any example herein, particularly examples 1-82, further comprising at least one tensile stiffening fiber for limiting bending of the delivery catheter in a direction opposite the circumferential location of the at least one tensile stiffening fiber.

Example 84: The delivery catheter according to any example herein, particularly example 83, wherein the least one tensile stiffening fiber extends along a portion of an entire length of the outer jacket.

Example 85: The delivery catheter according to any example herein, particularly examples 83-84, wherein the least one tensile stiffening fiber extends along an entire length of the outer jacket.

Example 86: The delivery catheter according to any example herein, particularly examples 83-85, further including a braided layer disposed within (and/or along) at least a portion of the outer jacket, wherein the at least one tensile stiffening fiber is provided along the braided layer.

Example 87: The delivery catheter according to any example herein, particularly example 86, wherein the at least one tensile stiffening fiber is woven into a portion of an entire length of the braided layer.

Example 88: The delivery catheter according to any example herein, particularly example 86, wherein the at least one tensile stiffening fiber is woven into the braided layer along an entire length of the braided layer.

Example 89: The delivery catheter according to any example herein, particularly example 86, wherein the at least one tensile stiffening fiber is not woven into a first portion of the braided layer, wherein the at least one fiber is woven into a second portion of the braided layer.

Example 90: The delivery catheter according to any example herein, particularly example 89, wherein the second portion of the braided layer (for example, woven tensile stiffening fiber) is provided at a distal tip portion of the delivery catheter such that the tensile stiffening fiber is not woven into the braided layer at a location corresponding to the coil layer.

Example 91: The delivery catheter according to any example herein, particularly examples 83-90, wherein a distal end of the at least one tensile stiffening fiber is fixedly coupled to the distal end of the delivery catheter.

Example 92: The delivery catheter according to any example herein, particularly examples 83-90, wherein a distal end of the at least one tensile stiffening fiber is fixedly coupled to a braided layer at a distal end of the braided layer.

Example 93: The delivery catheter according to any example herein, particularly examples 83-90, wherein a distal end of the at least one tensile stiffening fiber is fixedly coupled to the outer jacket at the distal end of the outer jacket.

Example 94: The delivery catheter according to any example herein, particularly examples 83-93, wherein the at least one tensile stiffening fiber is composed of at least one of a polymer, a metal, a composite.

Example 95: The delivery catheter according to any example herein, particularly example 94, wherein the at least one tensile stiffening fiber comprises a polyimide type polymer.

Example 96: The delivery catheter according to any example herein, particularly example 94, wherein the at least one tensile stiffening fiber comprises Kevlar (for example, Technora®).

Example 97: The delivery catheter according to any example herein, particularly example 94, wherein the at least one tensile stiffening fiber comprises liquid crystal polymer.

Example 98: The delivery catheter according to any example herein, particularly examples 83-97, wherein the at least one tensile stiffening fiber comprises a plurality of individual tensile stiffening fibers.

Example 99: The delivery catheter according to any example herein, particularly example 98, wherein the plurality of individual tensile stiffening fibers are equally spaced around the circumference of the delivery catheter (for example, every 60 degrees, 90 degrees, 120 degrees, 180 degrees).

Example 100: The delivery catheter according to any example herein, particularly example 98, wherein the plurality of individual tensile stiffening fibers are irregularly spaced around the circumference of the delivery catheter.

Example 101: The delivery catheter according to any example herein, particularly examples 83-100, wherein the at least one tensile stiffening fiber comprises six or less individual tensile stiffening fibers.

Example 102: The delivery catheter according to any example herein, particularly example 101, wherein the at least one tensile stiffening fiber comprises four or less individual tensile stiffening fibers.

Example 103: The delivery catheter according to any example herein, particularly example 102, wherein the at least one tensile stiffening fiber comprises one individual tensile stiffening fibers.

Example 104: The delivery catheter according to any example herein, particularly example 103, wherein the one individual tensile stiffening fiber is located at a neutral axis of the delivery catheter.

Example 105: The delivery catheter according to any example herein, particularly examples 1-104, wherein the elasticity of the outer jacket varies (for example, longitudinally) along a length of the outer jacket.

Example 106: The delivery catheter according to any example herein, particularly example 105, wherein the elasticity of the outer jacket increases between the proximal and distal end of the outer jacket such that the distal end of the delivery catheter is configured to conform to the aortic geometry of a patient.

Example 107: The delivery catheter according to any example herein, particularly examples 105-106, wherein the durometer of the outer jacket decreases between the proximal end and the distal end of the outer jacket.

Example 108: The delivery catheter according to any example herein, particularly examples 105-107, wherein various longitudinal segments of the outer jacket (for example, circumferential bands of material) are constructed from materials having varying durometer.

Example 109: The delivery catheter according to any example herein, particularly example 108, wherein the materials of the various longitudinal segments are reflowed together.

Example 110: The delivery catheter according to any example herein, particularly examples 1-109, wherein at least a portion of the outer jacket comprises an elastomer.

Example 111: The delivery catheter according to any example herein, particularly examples 1-110, wherein at least a portion of the outer jacket comprises a polyether block amide.

Example 112: The delivery catheter according to any example herein, particularly example 111, wherein the polyether block amide comprises Pebax.

Example 113: The delivery catheter according to any example herein, particularly examples 1-112, wherein at least a portion of the outer jacket comprises a polyimide (for example, Vestamid®).

Example 114: The delivery catheter according to any example herein, particularly examples 1-113, wherein a length of the second portion of the outer jacket is less than a length the first portion of the outer jacket.

Example 115: The delivery catheter according to any example herein, particularly examples 1-116, wherein the first portion of the outer jacket extends along at least a portion of the entire length of the body portion of the delivery catheter.

Example 116: The delivery catheter according to any example herein, particularly examples 1-115, wherein the first inner diameter of the first portion of the outer jacket is equal to an inner diameter of the central lumen of the coil layer.

Example 117: The delivery catheter according to any example herein, particularly examples 1-116, wherein the first inner diameter of the first portion of the outer jacket is less than an inner diameter of the central lumen of the coil layer.

Example 118: The delivery catheter according to any example herein, particularly examples 1-117, wherein the shoulder of the outer jacket further includes a taper.

Example 119: The delivery catheter according to any example herein, particularly examples 1-118, further including a ring having a proximal end and a distal end and defining a central lumen, wherein the ring is disposed within the cavity such that the proximal end of the coil layer is adjacent the distal end of the ring.

Example 120: The delivery catheter according to any example herein, particularly example 119, wherein the ring is partially disposed within the first portion of the outer jacket.

Example 121: The delivery catheter according to any example herein, particularly example 119, wherein the ring is fully disposed within the first portion of the outer jacket.

Example 122: The delivery catheter according to any example herein, particularly examples 119-121, wherein the ring includes a circumferential extrusion extending from the proximal end of the ring, wherein a thickness of the circumferential extrusion is less than a thickness of the ring.

Example 123: The delivery catheter according to any example herein, particularly example 122, wherein an inner diameter of the circumferential extrusion is greater than an inner diameter of the ring.

Example 124: The delivery catheter according to any example herein, particularly examples 122-123, wherein the circumferential extrusion is at least partially disposed within the first portion of the outer jacket.

Example 125: The delivery catheter according to any example herein, particularly examples 122-124, wherein the circumferential extrusion is fully disposed within the first portion of the outer jacket.

Example 126: The delivery catheter according to any example herein, particularly examples 122-125, wherein the circumferential extrusion is received in a corresponding shaped slot (for example, a circumferential slot) provided in the first portion of the outer jacket.

Example 127: The delivery catheter according to any example herein, particularly examples 119-126, wherein the ring further includes at least one opening.

Example 128: The delivery catheter according to any example herein, particularly example 127, wherein the at least one opening is provided on the circumferential extrusion.

Example 129: The delivery catheter according to any example herein, particularly examples 127-128, wherein the opening includes a plurality of openings equally spaced around the ring.

Example 130: The delivery catheter according to any example herein, particularly examples 120-129, wherein the ring is reflowed with the first portion of the outer jacket.

Example 131: The delivery catheter according to any example herein, particularly example 130, wherein the reflowed material extends through the at least one opening provided in the ring.

Example 132: The delivery catheter according to any example herein, particularly examples 120-131, wherein at least a portion of the ring is embedded in the first portion of the outer jacket.

Example 133: The delivery catheter according to any example herein, particularly examples 120-132, wherein the ring is composed of at least one of a polymer, a metal, or a composite.

Example 134: The delivery catheter according to any example herein, particularly example 133, wherein the ring is composed of stainless steel.

Example 135: The delivery catheter according to any example herein, particularly examples 1-134, further comprising a pull wire, the pull wire being coupled to the outer jacket.

Example 136: The delivery catheter according to any example herein, particularly example 135, wherein the pull wire is coupled to the distal end of the outer jacket.

Example 137: The delivery catheter according to any example herein, particularly example 135, wherein the pull wire is disposed within the outer jacket.

Example 138: A flexible delivery catheter comprising: an outer jacket, having a proximal end, a distal end, an inner surface and an outer surface, and defining a central lumen extending longitudinally between the proximal end and the distal end; a hypotube disposed at least partially within the central lumen of the outer jacket, the hypotube having a proximal end, a distal end, an inner surface, an outer surface, and a plurality of cut-outs which extend between the inner surface and the outer surface of the hypotube such that the hypotube can bend omnidirectionally, and a coupler provided at a distal end of the outer jacket, the coupler having a proximal end, a distal end, an outer surface, and defining a central lumen extending longitudinally between the proximal end and the distal end, wherein the outer jacket includes a first portion with a first inner diameter and a second portion with a second inner diameter, first portion adjacent the proximal end of the outer jacket, the second portion adjacent the distal end of the outer jacket, and the second inner diameter being greater than the first inner diameter, wherein the outer jacket further includes a shoulder disposed between the first and second portions, wherein the coupler and the inner surface of the outer jacket form a cavity (and the distal end of the inner liner when included), the hypotube is disposed within the cavity such that the hypotube abuts the inner surface of the second portion of the outer jacket.

Example 139: The delivery catheter according to any example herein, particularly example 138, wherein the delivery catheter is omnidirectionally flexible and configured to bend in any direction away from the longitudinal axis of the delivery catheter.

Example 140: The delivery catheter according to any example herein, particularly examples 138 or 139, wherein the delivery catheter includes a distal tip portion extending from the distal end of the delivery catheter along a length of the delivery catheter and an elongated body portion extending from the proximal end of the delivery catheter to the distal tip portion of the delivery catheter.

Example 141: The delivery catheter according to any example herein, particularly example 140, wherein the distal tip portion corresponds to the axial location of the cavity.

Example 142: The delivery catheter according to any example herein, particularly examples 138-141, wherein an outer diameter of the distal tip portion corresponds with an outer diameter of the elongated body portion.

Example 143: The delivery catheter according to any example herein, particularly examples 140-142, wherein an outer diameter of the distal tip portion is greater than an outer diameter of the elongated body portion.

Example 144: The delivery catheter according to any example herein, particularly examples 140-142, wherein the outer diameter of the elongated body portion tapers radially outwardly to the outer diameter of the distal tip portion.

Example 145: The delivery catheter according to any example herein, particularly examples 140-144, wherein an outer diameter of the outer jacket, including the distal tip portion and the elongated body portion, ranges between about 5 mm and about 6.0 mm.

Example 146: The delivery catheter according to any example herein, particularly examples 140-145, wherein an outer diameter of the outer jacket, including the distal tip portion and the elongated body portion ranges between about 4.5 mm and about 6 mm.

Example 147: The delivery catheter according to any example herein, particularly examples 140-146, wherein an outer diameter of the outer jacket, including the distal tip portion and the elongated body portion is about 5.4 mm (0.213 inches).

Example 148: The delivery catheter according to any example herein, particularly examples 138-147, wherein the first inner diameter of the outer jacket and/or the hypotube ranges between about 3.5 mm and about 5 mm.

Example 149: The delivery catheter according to any example herein, particularly examples 138-148, wherein the first inner diameter of the outer jacket and/or the hypotube ranges between about 4.0 mm and about 4.5 mm.

Example 150: The delivery catheter according to any example herein, particularly examples 138-149, wherein the first inner diameter of the outer jacket and/or the hypotube is about 4.8 mm.

Example 151: The delivery catheter according to any example herein, particularly examples 138-150, wherein the second inner diameter of the outer jacket ranges between about 4.6 mm and about 5.4 mm.

Example 152: The delivery catheter according to any example herein, particularly examples 138-151, wherein an outer diameter of the hypotube is less than the second inner diameter of the outer jacket.

Example 153: The delivery catheter according to any example herein, particularly examples 138-152, wherein an outer diameter of the hypotube corresponds with the second inner diameter of the outer jacket.

Example 154: The delivery catheter according to any example herein, particularly examples 138-153, wherein an outer diameter of the hypotube is less than the second inner diameter of the outer jacket.

Example 155: The delivery catheter according to any example herein, particularly examples 138-154, wherein an outer diameter of the hypotube ranges between about 4.6 mm and about 5.4 mm.

Example 156: The delivery catheter according to any example herein, particularly examples 138-155, wherein a length of the hypotube corresponds to a length of the second portion of the outer jacket, wherein the length of the hypotube is measured in a direction along the longitudinal axis of the delivery catheter.

Example 157: The delivery catheter according to any example herein, particularly examples 138-156, wherein a length of the hypotube is less than a length of the cavity.

Example 158: The delivery catheter according to any example herein, particularly examples 138-157, wherein a length of the hypotube corresponds to a length of the distal tip portion.

Example 159: The delivery catheter according to any example herein, particularly examples 138-158, wherein the hypotube has a length less than 305 mm (12 inches), wherein the length of the distal tip portion is measured in a direction along the longitudinal axis of the delivery catheter.

Example 160: The delivery catheter according to any example herein, particularly examples 138-159, wherein the hypotube has a length less than 254 mm (10 inches).

Example 161: The delivery catheter according to any example herein, particularly examples 138-160, wherein the length of the hypotube ranges between 76 mm (3 inches) and 254 mm (10 inches).

Example 162: The delivery catheter according to any example herein, particularly examples 138-161, wherein the length of the hypotube ranges between 178 mm (7 inches) and 254 mm (10 inches).

Example 163: The delivery catheter according to any example herein, particularly examples 138-162, wherein the length of the hypotube is about 215.9 mm (8.5 inches).

Example 164: The delivery catheter according to any example herein, particularly examples 138-163, wherein an overall length of the outer jacket ranges between about 762 mm (30 inches) to about 1178 mm (70 inches).

Example 165: The delivery catheter according to any example herein, particularly examples 138-164, wherein an overall length of the outer jacket ranges between about 889 mm (35 inches) to about 1397 mm (55 inches).

Example 166: The delivery catheter according to any example herein, particularly examples 138-165, wherein an overall length of the outer jacket is about 1320.8 mm (52 inches).

Example 167: The delivery catheter according to any example herein, particularly examples 138-166, wherein the hypotube resists axial compression and tension applied to the delivery catheter, and the hypotube facilitates bending of the delivery catheter in a direction away from the longitudinal axis of the delivery catheter.

Example 168: The delivery catheter according to any example herein, particularly examples 138-167, wherein the hypotube is disposed in the cavity such that the proximal end of the hypotube abuts the shoulder of the outer jacket, and the distal end of the hypotube abuts a radially extending protrusion of the coupler.

Example 169: The delivery catheter according to any example herein, particularly examples 138-168, wherein the hypotube includes cut-outs along a majority of its axial length.

Example 170: The delivery catheter according to any example herein, particularly examples 138-169, wherein each of the plurality of cut-outs are disposed in circumferential rows along an axial length of the hypotube.

Example 171: The delivery catheter according to any example herein, particularly example 170, wherein a first distal most row of the rows of cut-outs is offset from the distal end of the hypotube by 3.81 mm (0.150 inches).

Example 172: The delivery catheter according to any example herein, particularly examples 170-171, wherein each row comprises at least two cut-outs.

Example 173: The delivery catheter according to any example herein, particularly examples 170-172, wherein each row comprises at least four cut-outs.

Example 174: The delivery catheter according to any example herein, particularly examples 170-173, each of the plurality of cut-outs of in the same row are distributed symmetrically about the circumference of the hypotube.

Example 175: The delivery catheter according to any example herein, particularly examples 170-174, wherein each of the plurality of cut-outs of in the same row are distributed asymmetrically about the circumference of the hypotube.

Example 176: The delivery catheter according to any example herein, particularly examples 170-175, wherein the plurality of cut-outs in a row are circumferentially offset from the plurality of cut-outs in an adjacent row.

Example 177: The delivery catheter according to any example herein, particularly examples 138-176, wherein each of the plurality of cut-outs comprises a circumferential slit and a semicircular hole (for example, for relieving stressing during bending of the hypotube).

Example 178: The delivery catheter according to any example herein, particularly examples 138-177, wherein each of the plurality of cut-outs comprises a circumferential slit and a semicircular hole provided at a circumferential end of the slit.

Example 179: The delivery catheter according to any example herein, particularly examples 138-178, wherein each of the plurality of cut-outs comprises a circumferential slit and semicircular holes provided at each circumferential end of the slit.

Example 180: The delivery catheter according to any example herein, particularly examples 178-179, wherein a radius of the semicircular hole ranges between about 0.1016 mm (0.004 inches) and about 0.762 mm (0.030 inches).

Example 181: The delivery catheter according to any example herein, particularly examples 175-180, wherein a radius of the semicircular hole is about 0.1524 mm (0.006 inches).

Example 182: The delivery catheter according to any example herein, particularly examples 178-181, wherein each of the slits has a circumferential length ranging between about 3.0 mm and about 4.0 mm.

Example 183: The delivery catheter according to any example herein, particularly examples 178-182, wherein each of the slits has a circumferential length of about 3.3 mm (0.130 inches).

Example 184: The delivery catheter according to any example herein, particularly examples 178-183, wherein each of the slits has an axial width ranging between about 0.010 mm (0.0004 inches) and about 0.025 mm (0.0010 inches).

Example 185: The delivery catheter according to any example herein, particularly examples 178-184, wherein each of the slits has an axial width of about 0.018 mm (0.0007 inches).

Example 186: The delivery catheter according to any example herein, particularly examples 138-185, wherein the hypotube has a thickness ranging between about 0.0762 mm (0.0030 inches) and about 0.381 mm (0.0150 inches).

Example 187: The delivery catheter according to any example herein, particularly examples 138-186, wherein the hypotube has a thickness of about 0.0070 inches.

Example 188: The delivery catheter according to any example herein, particularly examples 138-187, wherein the hypotube has an outer diameter ranging between about 0.2000 inches and about 0.3000 inches.

Example 189: The delivery catheter according to any example herein, particularly examples 138-188, wherein the hypotube has an outer diameter of about 0.2030 inches.

Example 190: The delivery catheter according to any example herein, particularly examples 138-189, wherein the hypotube has an inner diameter ranging between about 0.1000 and about 0.2000 inches.

Example 191: The delivery catheter according to any example herein, particularly examples 138-190, wherein the hypotube has an inner diameter of about 0.1890 inches.

Example 192: The delivery catheter according to any example herein, particularly examples 138-191, wherein an overall length of the hypotube corresponds to an overall length of the cavity of the delivery catheter.

Example 193: The delivery catheter according to any example herein, particularly examples 138-192, wherein an overall length of the hypotube is less than an overall length of the cavity of the delivery catheter.

Example 194: The delivery catheter according to any example herein, particularly examples 138-193, wherein the hypotube is composed of at least one of a polymer, a metal, a composite.

Example 195: The delivery catheter according to any example herein, particularly example 194, wherein the hypotube is composed of stainless steel.

Example 196: The delivery catheter of anyone according to any example herein, particularly examples 135-195, further including a delivery capsule with a central lumen, where the coupler couples the delivery capsule to the outer jacket.

Example 197: The delivery catheter according to any example herein, particularly example 196, wherein the central lumen of the delivery capsule is sized and configured to receive a prosthetic heart valve in a crimped or non-expanded configuration therein.

Example 198: The delivery catheter according to any example herein, particularly examples 138-197, further including a prosthetic heart valve in a crimped or non-expanded configuration within the central lumen of the delivery capsule.

Example 199: The delivery catheter according to any example herein, particularly examples 138-198, wherein an outer diameter of the delivery catheter at the delivery capsule is greater than an outer diameter of the outer jacket.

Example 200: The delivery catheter according to any example herein, particularly examples 138-199, wherein the outer diameter of the delivery capsule ranges between about 7 mm and about 9 mm.

Example 201: The delivery catheter according to any example herein, particularly examples 196-200, wherein the coupler is fixedly coupled to at least one of the outer jacket and/or the delivery capsule.

Example 202: The delivery catheter according to any example herein, particularly examples 196-201, wherein the coupler is coupled to the outer jacket by at least one of a press fit, an interference fit, a snap fit, a mechanical fastener, a weld and an adhesive, wherein the coupler is coupled to the delivery capsule by at least one of a press fit, an interference fit, a snap fit, a mechanical fastener, a weld and an adhesive.

Example 203: The delivery catheter according to any example herein, particularly examples 196-202, wherein the outer surface of coupler includes a recess for receiving the delivery capsule such that an outer diameter of the delivery capsule corresponds with an outer diameter of the coupling segment.

Example 204: The delivery catheter according to any example herein, particularly examples 196-203, wherein the outer surface of the coupler adjacent the outer jacket includes a decreasing taper between a larger outer diameter delivery capsule and a reduced diameter outer jacket.

Example 205: The delivery catheter according to any example herein, particularly examples 138-204, where the inner diameter (for example, central lumen) of the coupler includes a shoulder, wherein the distal end of the hypotube abuts the shoulder securing the hypotube between the shoulder of the coupler and the shoulder of the outer jacket.

Example 206: The delivery catheter according to any example herein, particularly example 205, wherein the shoulder of the coupler comprises a radially extending protrusion extending from the central lumen of the coupler.

Example 207: The delivery catheter according to any example herein, particularly examples 205-206, wherein a diameter of the radially extending protrusion corresponds to a diameter of the central lumen of the hypotube.

Example 208: The delivery catheter according to any example herein, particularly examples 196-207, wherein the central lumen of the coupler adjacent the delivery capsule includes an increasing taper between smaller diameter central lumen of the coupling segment and a larger outer diameter central lumen of the delivery capsule.

Example 209: The delivery catheter according to any example herein, particularly examples 138-208, wherein the central lumen of the coupler includes a cylindrical portion adjacent the proximal end, an increasing taper portion adjacent the distal end, and a radially extending protrusion between the cylindrical portion and the increasing taper portion, wherein a diameter of the radially extending protrusion is less than a diameter of the cylindrical portion.

Example 210: The delivery catheter according to any example herein, particularly examples 138-209, wherein the central lumen of the coupler includes at least one axially extending recess, wherein the distal end of the hypotube further includes at least one axially extending protrusion such that the at least one axially extending protrusion can be disposed within the at least on axially extending recess.

Example 211: The delivery catheter according to any example herein, particularly example 210, where the inner diameter (for example, central lumen) of the coupler includes a shoulder and wherein the axial extending recess is provided in the shoulder such that when the axially extending protrusion is disposed within the axially extending recess, the hypotube is fixedly coupled to the coupler.

Example 212: The delivery catheter according to any example herein, particularly examples 210-211, wherein the at least one axially extending recess includes a plurality of axially extending recesses, and the at least one axially extending protrusion includes a plurality of axially extending protrusions.

Example 213: The delivery catheter according to any example herein, particularly example 212, wherein the at least one axially extending recess includes two axially extending recesses, and the at least one axially extending protrusion includes two axially extending protrusions.

Example 214: The delivery catheter according to any example herein, particularly examples 212-213, wherein the plurality of axially extending recesses and the plurality of axially extending protrusions are spaced evenly around the circumference of the coupler and hypotube, respectively.

Example 215: The delivery catheter according to any example herein, particularly examples 212-213, wherein the plurality of axially extending recesses and the plurality of axially extending protrusions are spaced unevenly around the circumference of the coupler and hypotube, respectively.

Example 216: The delivery catheter according to any example herein, particularly examples 138-215, further including a braided layer having a proximal end and a distal end and comprising a plurality of fibers disposed in a braided pattern and defining a central lumen having an inner surface and an outer surface.

Example 217: The delivery catheter according to any example herein, particularly example 216, wherein the braided layer is at least partially disposed within the outer jacket.

Example 218: The delivery catheter according to any example herein, particularly examples 216-217, wherein the braided layer extends along an entire length of the outer jacket.

Example 219: The delivery catheter according to any example herein, particularly examples 216-218, wherein the braided layer extends along a portion of an entire length of the outer jacket.

Example 220: The delivery catheter according to any example herein, particularly example 219, wherein the delivery catheter includes a distal tip portion extending from the proximal end of the delivery capsule along a length of the delivery catheter and an elongated body portion extending from the proximal end of the delivery capsule to the distal tip portion of the delivery catheter, wherein the braided layer extends along the elongated body portion.

Example 221: The delivery catheter according to any example herein, particularly example 220, wherein the braided layer extends along at least a portion of the distal tip portion that corresponds to a proximal end of the hypotube.

Example 222: The delivery catheter according to any example herein, particularly examples 220-221, wherein the braided layer extends along at least a portion of the distal tip portion and a corresponding length of the hypotube.

Example 223: The delivery catheter according to any example herein, particularly examples 216-222, wherein the braided layer extends into the first portion of the outer jacket.

Example 224: The delivery catheter according to any example herein, particularly examples 216-223, wherein the braided layer comprises of a plurality of interwoven fibers.

Example 225: The delivery catheter according to any example herein, particularly example 224, wherein each of the plurality of interwoven fibers has a diameter ranging between about 0.002 inches and about 0.006 inches.

Example 226: The delivery catheter according to any example herein, particularly example 225, wherein each of the plurality of interwoven fibers has a diameter ranging between about 0.003 inches and about 0.005 inches.

Example 227: The delivery catheter according to any example herein, particularly example 226, wherein each of the plurality of interwoven fibers has a diameter of about 0.004 inches.

Example 228: The delivery catheter according to any example herein, particularly examples 216-227, wherein the plurality of interwoven fibers comprises about 10 to about 20 individual fibers.

Example 229: The delivery catheter according to any example herein, particularly example 228, wherein the plurality of interwoven fibers comprises 16 individual fibers.

Example 230: The delivery catheter according to any example herein, particularly examples 216-229, wherein the braided layer has a braid density between about 20 to about 30 picks per inch.

Example 231: The delivery catheter according to any example herein, particularly examples 216-230, wherein the braid density varies along the axial length of the catheter.

Example 232: The delivery catheter according to any example herein, particularly example 216-231, wherein the braid density of the braided layer is about 25 picks per inch.

Example 233: The delivery catheter according to any example herein, particularly examples 216-232, wherein the braided layer comprises of a plurality of interwoven fibers, wherein each of the plurality of interwoven fibers have a rectilinear or curvilinear shape in cross section.

Example 234: The delivery catheter according to any example herein, particularly examples 216-233, wherein the braided layer comprises of a plurality of interwoven fibers, wherein the plurality of interwoven fibers are composed of at least one of a polymer, a metal, a composite.

Example 235: The delivery catheter according to any example herein, particularly example 234, wherein the plurality of interwoven fibers includes stainless steel wire.

Example 236: The delivery catheter according to any example herein, particularly example 234, wherein the plurality of interwoven fibers is composed of a Liquid Crystal Polymer.

Example 237: The delivery catheter according to any example herein, particularly example 234, wherein the plurality of interwoven fibers is composed of a Kevlar (for example, Technora®).

Example 238: The delivery catheter according to any example herein, particularly examples 138-236, further comprising at least one tensile stiffening fiber for limiting bending of the delivery catheter in a direction opposite the circumferential location of the at least one tensile stiffening fiber.

Example 239: The delivery catheter according to any example herein, particularly example 238, wherein the least one tensile stiffening fiber extends along a portion of an entire length of the outer jacket.

Example 240: The delivery catheter according to any example herein, particularly examples 238-239, wherein the least one tensile stiffening fiber extends along an entire length of the outer jacket.

Example 241: The delivery catheter according to any example herein, particularly examples 238-240, further including a braided layer disposed within (and/or along) at least a portion of the outer jacket, wherein the at least one tensile stiffening fiber is provided along the braided layer.

Example 242: The delivery catheter according to any example herein, particularly example 241, wherein the at least one tensile stiffening fiber is woven into a portion of an entire length of the braided layer.

Example 243: The delivery catheter according to any example herein, particularly example 242, wherein the at least one tensile stiffening fiber is woven into the braided layer along an entire length of the braided layer.

Example 244: The delivery catheter according to any example herein, particularly example 243, wherein the at least one tensile stiffening fiber is not woven into a first portion of the braided layer, wherein the at least one fiber is woven into a second portion of the braided layer.

Example 245: The delivery catheter according to any example herein, particularly example 244, wherein the second portion of the braided layer (for example, woven tensile stiffening fiber) is provided at a distal tip portion of the delivery catheter such that the tensile stiffening fiber is not woven into the braided layer at a location corresponding to the hypotube.

Example 246: The delivery catheter according to any example herein, particularly examples 238-245, wherein a distal end of the at least one tensile stiffening fiber is fixedly coupled to the distal end of the delivery catheter.

Example 247: The delivery catheter according to any example herein, particularly examples 238-245, wherein a distal end of the at least one tensile stiffening fiber is fixedly coupled to a braided layer at a distal end of the braided layer.

Example 248: The delivery catheter according to any example herein, particularly examples 238-245, wherein a distal end of the at least one tensile stiffening fiber is fixedly coupled to the outer jacket at the distal end of the outer jacket.

Example 249: The delivery catheter according to any example herein, particularly examples 238-248, wherein the at least one tensile stiffening fiber is composed of at least one of a polymer, a metal, a composite.

Example 250: The delivery catheter according to any example herein, particularly example 249, wherein the at least one tensile stiffening fiber comprises a polyimide type polymer.

Example 251: The delivery catheter according to any example herein, particularly example 259, wherein the at least one tensile stiffening fiber comprises Kevlar (for example, Technora®).

Example 252: The delivery catheter according to any example herein, particularly example 249, wherein the at least one tensile stiffening fiber comprises a Liquid Crystal polymer.

Example 253: The delivery catheter according to any example herein, particularly examples 238-252, wherein the at least one tensile stiffening fiber comprises a plurality of individual tensile stiffening fibers.

Example 254: The delivery catheter according to any example herein, particularly example 253, wherein the plurality of individual tensile stiffening fibers are equally spaced around the circumference of the delivery catheter (for example, every 60 degrees, 90 degrees, 120 degrees, 180 degrees).

Example 255: The delivery catheter according to any example herein, particularly example 254, wherein the plurality of individual tensile stiffening fibers are irregularly spaced around the circumference of the delivery catheter.

Example 256: The delivery catheter according to any example herein, particularly examples 238-255, wherein the at least one tensile stiffening fiber comprises six or less individual tensile stiffening fibers.

Example 257: The delivery catheter according to any example herein, particularly example 256, wherein the at least one tensile stiffening fiber comprises four or less individual tensile stiffening fibers.

Example 258: The delivery catheter according to any example herein, particularly example 257, wherein the at least one tensile stiffening fiber comprises one individual tensile stiffening fibers.

Example 259: The delivery catheter according to any example herein, particularly example 258, wherein the one individual tensile stiffening fiber is located at a neutral axis of the delivery catheter.

Example 260: The delivery catheter according to any example herein, particularly examples 138-259, wherein the elasticity of the outer jacket varies (for example, longitudinally) along a length of the outer jacket.

Example 261: The delivery catheter according to any example herein, particularly example 260, wherein the elasticity of the outer jacket increases between the proximal and distal end of the outer jacket such that the distal end of the delivery catheter is configured to conform to the aortic geometry of a patient.

Example 262: The delivery catheter according to any example herein, particularly examples 260-261, wherein the durometer of the outer jacket decreases between the proximal end and the distal end of the outer jacket.

Example 263: The delivery catheter according to any example herein, particularly examples 260-262, wherein various longitudinal segments of the outer jacket (for example, circumferential bands of material) are constructed from materials having varying durometer.

Example 264: The delivery catheter according to any example herein, particularly example 263, wherein the materials of the various longitudinal segments are reflowed together.

Example 265: The delivery catheter according to any example herein, particularly examples 138-264, wherein at least a portion of the outer jacket comprises an elastomer.

Example 266: The delivery catheter according to any example herein, particularly examples 138-265, wherein at least a portion of the outer jacket comprises a polyether block amide.

Example 267: The delivery catheter according to any example herein, particularly example 266, wherein the polyether block amide comprises Pebax.

Example 268: The delivery catheter according to any example herein, particularly examples 138-267, wherein at least a portion of the outer jacket comprises a polyimide (for example, Vestamid®).

Example 269: The delivery catheter according to any example herein, particularly examples 138-268, wherein a length of the second portion of the outer jacket is less than a length of the first portion of the outer jacket.

Example 270: The delivery catheter according to any example herein, particularly examples 138-269, wherein the first portion of the outer jacket extends along at least a portion of the entire length of the body portion of the delivery catheter.

Example 271: The delivery catheter according to any example herein, particularly examples 138-270, wherein the first inner diameter of the outer jacket is equal to an inner diameter of the central lumen of the coil layer.

Example 272: The delivery catheter according to any example herein, particularly examples 138-271, wherein the first inner diameter of the outer jacket is less than an inner diameter of the central lumen of the hypotube.

Example 273: The delivery catheter according to any example herein, particularly examples 138-272, wherein the shoulder of the outer jacket further includes at least one axially extending recess that corresponds with an at least one axially extending protrusion of the hypotube.

Example 274: The delivery catheter according to any example herein, particularly examples 138-273, further including a ring having a proximal end, a distal end, and an outer surface and defining a central lumen, wherein the ring is disposed within the cavity such that the proximal end of the hypotube abuts the distal end of the ring.

Example 275: The delivery catheter according to any example herein, particularly example 274, wherein the ring is partially disposed within the first portion outer jacket.

Example 276: The delivery catheter according to any example herein, particularly example 274, wherein the ring is fully disposed within the first portion of the outer jacket.

Example 277: The delivery catheter according to any example herein, particularly examples 274-276, wherein the ring includes a circumferential extrusion extending from the proximal end of the ring, wherein a thickness of the circumferential extrusion is less than a thickness of ring.

Example 278: The delivery catheter according to any example herein, particularly example 277, wherein an inner diameter of the circumferential extrusion is greater than an inner diameter of the ring.

Example 279: The delivery catheter of any one according to any example herein, particularly examples 277-278, wherein the circumferential extrusion is at least partially disposed within the first portion of the outer jacket.

Example 280: The delivery catheter according to any example herein, particularly examples 277-279, wherein the circumferential extrusion is fully disposed within the first portion of the outer jacket.

Example 281: The delivery catheter according to any example herein, particularly examples 277-280, wherein the circumferential extrusion is received in a corresponding shaped slot (for example, a circumferential slot) provided in the first portion of the outer jacket.

Example 282: The delivery catheter according to any example herein, particularly examples 274-281, wherein the ring further includes at least one opening.

Example 283: The delivery catheter according to any example herein, particularly example 282, wherein the opening is provided on the circumferential extrusion.

Example 284: The delivery catheter according to any example herein, particularly examples 282-283, wherein the opening includes a plurality of openings equally spaced around the ring.

Example 285: The delivery catheter according to any example herein, particularly examples 274-284, wherein the ring is reflowed with the outer jacket.

Example 286: The delivery catheter according to any example herein, particularly example 285, wherein the reflowed material extends through at least one opening provided in the ring.

Example 287: The delivery catheter according to any example herein, particularly examples 274-286, wherein at least a portion of the ring is embedded in the outer jacket.

Example 288: The delivery catheter according to any example herein, particularly examples 274-287, wherein the ring is composed of at least one of a polymer, a metal, a composite.

Example 289: The delivery catheter according to any example herein, particularly example 288, wherein the ring is composed of stainless steel.

Example 290: The delivery catheter according to any example herein, particularly examples 274-289, wherein the distal end of the ring defines at least one axially extending recess, wherein the proximal end of the hypotube further includes at least one axially extending protrusion such that the at least one axially extending protrusion can be disposed within the at least one axially extending recess.

Example 291: The delivery catheter according to any example herein, particularly examples 274-290, wherein the distal end of the ring defines at least one axially extending projection, wherein the proximal end of the hypotube further includes at least one axially extending recess such that the at least one axially extending protrusion can be disposed within the at least one axially extending recess.

Example 292: The delivery catheter according to any example herein, particularly examples 290-291, wherein the at least one axially extending recess includes a plurality of axially extending recesses, and the at least one axially extending protrusion includes a plurality of axially extending protrusions.

Example 293: The delivery catheter according to any example herein, particularly example 292, wherein the at least one axially extending recess includes two axially extending recesses, and the at least one axially extending protrusion includes two axially extending protrusions.

Example 294: The delivery catheter according to any example herein, particularly examples 292-293, wherein the plurality of axially extending recesses and the plurality of axially extending protrusions are spaced evenly around the circumference of the ring and hypotube.

Example 295: The delivery catheter according to any example herein, particularly examples 292-293, wherein the plurality of axially extending recess and the plurality of axially extending protrusion are spaced unevenly around the circumference of the ring and hypotube.

Example 296: The delivery catheter according to any example herein, particularly examples 138-295, further comprising a pull wire, the pull wire being coupled to the outer jacket.

Example 297: The delivery catheter according to any example herein, particularly example 296, wherein the pull wire is coupled to the distal end of the outer jacket.

Example 298: The delivery catheter according to any example herein, particularly example 296, wherein the pull wire is disposed within the outer jacket.

Example 299: A method of making a flexible delivery catheter comprising: forming an outer jacket, having a proximal end, a distal end, an inner surface, an outer surface, a first portion with a first inner diameter, a second portion with a second inner diameter, and a shoulder between the first portion and the second portion and defining a central lumen extending longitudinally between the proximal end and the distal end, the first portion adjacent the proximal end of the outer jacket, the second portion adjacent the distal end of the outer jacket, and the second inner diameter being greater than the first inner diameter; forming a coil layer comprising a coil winding and having a proximal end, a distal end, an inner surface, and an outer surface, wherein the coil winding extends helically about a longitudinal axis and defines a central lumen between the proximal end and the distal end; providing a coupler having a proximal end, a distal end, and a radially extending protrusion and defining a central lumen extending longitudinally between the proximal end and the distal end; disposing the coil layer within the central lumen of the outer jacket such that the coil layer abuts the inner surface of the second portion of the outer jacket and the shoulder of the outer jacket; coupling the coupler to the distal end of the outer jacket such that the coupler abuts the distal end of the outer jacket and the coil layer is secured within in the second portion of the outer jacket between the proximal end of the coupler and the shoulder of the outer jacket.

Example 300: The method according to any example herein, particularly example 299, further including forming a braided layer having a proximal end and a distal end and comprising a plurality of wires disposed in a braided pattern and defining a central lumen having an inner surface and an outer surface, and disposing the braided layer within the central lumen of between the outer jacket.

Example 301: The method according to any example herein, particularly example 300, wherein the braided layer is reflowed with the outer jacket.

Example 302: The method according to any example herein, particularly examples 299-301, wherein the coil layer includes a gap/spacing between adjacent turns of the coil winding.

Example 303: The method according to any example herein, particularly examples 299-301, wherein an outer surface of adjacent turns of the coiled winding contact along a circumferential length of the coiled winding.

Example 304: The method according to any example herein, particularly examples 299-302, wherein the outer jacket comprises a plurality of longitudinal segments (for example, circumferential bands of material) of varying elasticity.

Example 305: The method according to any example herein, particularly example 304, wherein each of the plurality of longitudinal segments are reflowed together with at least one other longitudinal segment.

Example 306: The method according to any example herein, particularly examples 299-305, further comprising coupling a tensile stiffening fiber to the braided layer.

Example 307: The method according to any example herein, particularly example 306, wherein coupling the tensile stiffening fiber to the braided layer further comprises weaving the tensile stiffening fiber into the braided layer.

Example 308: The method according to any example herein, particularly examples 306-307, further comprising coupling the tensile stiffening fiber to the outer jacket.

Example 309: The method according to any example herein, particularly examples 299-308, further including providing a ring having a proximal end, a distal end, and an outer surface and defining a central lumen, and disposing the ring within the cavity such that the proximal end of the coil layer abuts the distal end of the ring.

Example 310 The method according to any example herein, particularly example 309, further comprising reflowing the ring with the outer jacket such that the reflowed material of the outer jacket extends through an opening provided in the ring.

Example 311 A method of making a flexible delivery catheter comprising: forming an outer jacket, having a proximal end, a distal end, an inner surface, an outer surface, a first portion with a first inner diameter, a second portion with a second inner diameter, and a shoulder disposed between the first portion and the second portion and defining a central lumen extending longitudinally between the proximal end and the distal end, the first portion adjacent the proximal end of the outer jacket, the second portion adjacent the distal end of the outer jacket, and the second inner diameter being greater than the first inner diameter; forming a hypotube having a proximal end, a distal end, an inner surface, an outer surface, and a plurality of cut-outs which extend between the inner surface and the outer surface of the hypotube such that the hypotube can bend omnidirectionally; providing a coupler having a proximal end, a distal end, and a radially extending protrusion and defining a central lumen extending longitudinally between the proximal end and the distal end, disposing the hypotube within the central lumen of the outer jacket such that the hypotube abuts the inner surface of the second portion of the outer jacket and the shoulder of the outer jacket; coupling the coupler to the distal end of the outer jacket such that the coupler abuts the distal end of the outer jacket and the hypotube is secured within the second portion of the outer jacket between the proximal end of the coupler and the shoulder of the outer jacket.

Example 312: The method according to any example herein, particularly example 311, further including forming a braided layer having a proximal end and a distal end and comprising a plurality of wires disposed in a braided pattern and defining a central lumen having an inner surface and an outer surface, and disposing the braided layer within the central lumen of between the outer jacket.

Example 313: The method according to any example herein, particularly example 312, wherein the braided layer is reflowed with the outer jacket.

Example 314: The method according to any example herein, particularly examples 311-313, wherein the outer jacket comprises a plurality of longitudinal segments (for example, circumferential bands of material) of varying elasticity.

Example 315: The method according to any example herein, particularly example 314, wherein each of the plurality of longitudinal segments are reflowed together with at least one other longitudinal segment.

Example 316: The method according to any example herein, particularly examples 311-315, further comprising coupling a tensile stiffening fiber to the braided layer.

Example 317: The method according to any example herein, particularly example 316, wherein coupling the tensile stiffening fiber to the braided layer further comprises weaving the tensile stiffening fiber into the braided layer.

Example 318: The method according to any example herein, particularly examples 316-317, further comprising coupling the tensile stiffening fiber to the outer jacket.

Example 319: The method according to any example herein, particularly examples 311-318, further including providing a ring having a proximal end, a distal end, and an outer surface and defining a central lumen, and disposing the ring within the cavity such that the proximal end of the hypotube abuts the distal end of the ring.

Example 320: The method according to any example herein, particularly example 319, further comprising reflowing the ring with the outer jacket such that the reflowed material of the outer jacket extends through an opening provided in the ring.

Example 321: A method of deploying a prosthetic into a patient comprising: providing a delivery system comprising a flexible catheter having a proximal end and a distal end; advancing the catheter into a vessel of a patient; advancing the prosthetic from the proximal end of the catheter to the distal end of the catheter and into the patient.

Example 322: The method according to any example herein, particularly example 321, wherein the flexible catheter elastically conforms to the arterial structure of the patient.

Example 323: The method according to any example herein, particularly example 321 or 322, wherein the delivery system includes an outer jacket having an elasticity that varies (for example, longitudinally) along a length of the outer jacket.

Example 324: The method according to any example herein, particularly examples 321-323, wherein the flexible catheter further comprises a tensile stiffening fiber configured to allow omnidirectional bending while limiting limit axial tensile deformation within the patient.

Example 325: The method according to any example herein, particularly example any one according to any example herein, particularly examples 321-324, wherein the flexible catheter further comprises a hypotube disposed proximate the distal end of the delivery catheter configured to allow omnidirectional bending while limiting limit axial compression within the patient.

Example 326: The method according to any example herein, particularly example any one according to any example herein, particularly examples 321-324, wherein the catheter further comprises a coil disposed proximate the distal end of the delivery catheter configured to allow omnidirectional bending while limiting limit axial compression within the patient.

Example 327: The method according to any example herein, particularly examples 321-326, wherein the outer jacket further comprises a braided layer that has a longitudinally varying braid pattern configured to allow omnidirectional bending while limiting limit axial compression.

Example 328: The method according to any example herein, particularly example any one according to any example herein, particularly examples 321-327, wherein the prosthetic is a prosthetic heart valve.

Example 329: A flexible delivery catheter comprising: an outer jacket, having a proximal end, a distal end, an inner surface and an outer surface, and defining a central lumen extending longitudinally between the proximal end and the distal end, the outer jacket defining a flexible portion extending along a length of the outer jacket proximal the distal end; a pullwire mechanism coupled to the outer jacket for directing bending motion of the flexible portion of the outer jacket, the pullwire mechanism including: a pullwire extending along the outer jacket and coupled to the outer jacket at a location proximate the distal end of the outer jacket, and a knob coupled to the pullwire such that rotational movement of the knob causes the pullwire to move in a proximal or distal direction resulting in a corresponding bending motion of the flexible portion of the outer jacket; and a valve deployment assembly coupled to the outer jacket for directing proximal and distal movement of the outer jacket, the valve deployment assembly including: a housing with a central lumen extending therethrough, the central lumen including a threaded inner surface, an inner member extending within the central lumen of the housing, the inner member rotatably coupled to the housing and including a central lumen extending therethrough, a coupler fixedly coupled to the outer jacket and rotatably coupled to the housing, the coupler extending within the central lumen of the housing and the inner member, a proximal end portion of the coupler including a threaded outer surface that threadingly engages the threaded inner surface of the housing, wherein rotational movement of the housing causes the threaded inner surface of the housing to engage the threaded outer surface of the coupler resulting in a corresponding axial movement of the coupler within the housing and corresponding axial movement of the outer jacket in the proximal or distal direction.

Example 330: The flexible delivery catheter according to any example herein, particularly example 329, wherein the bending motion of the distal end of the outer jacket is in a direction transverse to the longitudinal axis of the delivery catheter.

Example 331: The flexible delivery catheter according to any example herein, particularly examples 329-330, wherein movement of the pullwire in the proximal direction, causes the distal end of the outer jacket (for example, flexible portion) to curve in a direction transverse to the longitudinal axis of the delivery catheter, wherein movement of the pullwire in the distal direction, causes the outer jacket (for example, flexible portion) to uncurve/straighten back toward the longitudinal axis of the delivery catheter.

Example 332: The flexible delivery catheter according to any example herein, particularly examples 329-331, wherein movement of the pullwire in the proximal direction increases a tensile load on outer sheath.

Example 333: The flexible delivery catheter according to any example herein, particularly examples 329-332, wherein, when the pullwire is not engaged (for example, no tension is provided on the pullwire) the flexible portion of the outer jacket can bend omnidirectionally any direction away from the longitudinal axis of the delivery catheter.

Example 334: The flexible delivery catheter according to any example herein, particularly examples 329-333, wherein the pullwire is not engaged (for example, no tension is provided on the pullwire) during advancement and positioning of the delivery catheter within the patient.

Example 335: The flexible delivery catheter according to any example herein, particularly examples 329-334, wherein a location of the flexible portion corresponds with the location of at least one of a coil layer and a hypotube disposed at least partially within the central lumen of the outer jacket, wherein the coil layer includes a coil winding and having a proximal end, a distal end, an inner surface, and an outer surface, wherein the coil winding extends helically about a longitudinal axis and defines a central lumen between the proximal end and the distal end, wherein the hypotube includes a proximal end, a distal end, an inner surface, an outer surface, and a plurality of cut-outs which extend between the inner surface and the outer surface of the hypotube such that the hypotube can bend omnidirectionally.

Example 336: The flexible delivery catheter according to any example herein, particularly example 335, wherein the coil layer is wound to resist axial compression and tension applied to the delivery catheter, and the coil layer facilitates bending of the delivery catheter in a direction away from the longitudinal axis of the delivery catheter, wherein the hypotube resists axial compression and tension applied to the delivery catheter, and the hypotube facilitates bending of the delivery catheter in a direction away from the longitudinal axis of the delivery catheter.

Example 337: The flexible delivery catheter according to any example herein, particularly examples 329-336, wherein the knob is rotatably coupled to the coupler of the valve deployment assembly.

Example 338: The flexible delivery catheter according to any example herein, particularly examples 329-337, wherein the coupler includes the proximal end portion received within the housing, a main body portion extending through and beyond a distal opening in the inner member and a distal opening in the housing, and a distal end portion extending distally from the main body portion, the distal end portion coupled to the pullwire mechanism.

Example 339: The flexible delivery catheter according to any example herein, particularly example 338, wherein the knob is rotatably coupled to the distal end portion of the coupler.

Example 340: The flexible delivery catheter according to any example herein, particularly examples 338-339, wherein the distal end portion of the coupler extends within a central lumen of the knob.

Example 341: The flexible delivery catheter according to any example herein, particularly examples 329-340, wherein the pullwire mechanism further includes a threaded sleeve operatively coupling the knob and the coupler, the threaded sleeve including a central lumen extending therethrough and a threaded outer surface, the threaded sleeve positioned on a distal end portion of the coupler such that the threaded sleeve is axially movably along the coupler (for example, distal end portion of the coupler extends through the central lumen of the threaded sleeve), the threaded outer surface of the threaded sleeve engaging a threaded inner surface of the knob, wherein rotational movement of the knob causes the threaded outer surface to engage the threaded inner surface of the knob resulting in a corresponding axial movement of the threaded sleeve along the coupler.

Example 342: The flexible delivery catheter according to any example herein, particularly example 341, wherein the pullwire mechanism further includes an anchor sleeve positioned on and axially movable along the distal end portion of the coupler between the threaded sleeve and the main body portion of the coupler, wherein the pullwire is fixedly coupled to the anchor sleeve, wherein rotational movement of the knob causes the threaded sleeve to engage the threaded inner surface of the knob resulting in a corresponding axial movement of the threaded sleeve and the anchor sleeve along the coupler, resulting in a corresponding movement of the pullwire in the proximal or distal direction.

Example 343: The flexible delivery catheter according to any example herein, particularly example 342, the anchor sleeve includes a central lumen extending therethrough and the distal end portion of the coupler extends through the central lumen of the anchor sleeve.

Example 344: The flexible delivery catheter according to any example herein, particularly examples 342-343, wherein the pullwire is fixedly coupled to the anchor sleeve by a mechanical fastener, a chemical fastener, or combinations thereof (for example, a press fit, an interference fit, a snap fit, a mechanical fastener, a weld, and an adhesive).

Example 345: The flexible delivery catheter according to any example herein, particularly examples 342-344, wherein the anchor sleeve includes an anchor post extending from an outer surface of the anchor sleeve, the pullwire fixedly coupled to the anchor post (for example, mechanical and/or a chemical fastener

Example 346: The flexible delivery catheter according to any example herein, particularly examples 342-345, wherein the pullwire extends through a longitudinally extending opening in the threaded sleeve to the anchor sleeve.

Example 347: The flexible delivery catheter according to any example herein, particularly examples 341-346, wherein the coupler includes an alignment feature extending longitudinally along an outer surface of the distal end portion, the alignment feature engages a corresponding alignment feature provided on an inner surface of at least one of the threaded sleeve and the anchor sleeve, wherein the engagement between the coupler alignment feature and at least one of the threaded sleeve alignment feature and the anchor sleeve alignment feature fixes the circumferential position of the threaded sleeve and/or anchor sleeve with respect to the coupler.

Example 348: The flexible delivery catheter according to any example herein, particularly example 347, wherein the coupler alignment feature includes a longitudinally extending ridge projecting from the outer surface of the coupler and the threaded sleeve alignment feature and/or the anchor sleeve alignment feature includes a correspondingly shaped recess provided on an inner surface of the threaded sleeve and/or anchor sleeve.

Example 349: The flexible delivery catheter according to any example herein, particularly example 347, wherein the coupler alignment feature includes a longitudinally extending groove recessed in the outer surface of the coupler and the threaded sleeve alignment feature and/or the anchor sleeve alignment feature includes a correspondingly shaped ridge projecting from an inner surface of the threaded sleeve and/or anchor sleeve.

Example 350: The flexible delivery catheter according to any example herein, particularly examples 329-349, further including a delivery capsule coupled to the distal of the outer jacket.

Example 351: The flexible delivery catheter according to any example herein, particularly example 350, wherein a delivery capsule coupler couples the delivery capsule to the outer jacket, the delivery capsule coupler is provided at a distal end of the outer jacket, the delivery capsule coupler having a proximal end, a distal end, and outer surface and defining a central lumen extending longitudinally between the proximal end and the distal end.

Example 352: The flexible delivery catheter according to any example herein, particularly examples 350-351, wherein a distal end of the pullwire is fixedly coupled to a mounting ring provided at a distal end of the outer jacket.

Example 353: The flexible delivery catheter according to any example herein, particularly example 352, wherein the mounting ring is provided between the distal end of the outer jacket and the delivery capsule (for example, between the distal end of the outer jacket and the delivery capsule coupler).

Example 354: The flexible delivery catheter according to any example herein, particularly examples 350-353, wherein a central lumen of the delivery capsule is sized and configured to receive a prosthetic device (for example, prosthetic heart valve) in a crimped or non-expanded configuration therein.

Example 355: The flexible delivery catheter according to any example herein, particularly examples 350-354, further including a prosthetic heart valve in a crimped or non-expanded configuration within the central lumen of the delivery capsule.

Example 356: The flexible delivery catheter according to any example herein, particularly examples 350-355, wherein rotational movement of the housing causes the threaded inner surface of the housing to engage the threaded outer surface of the coupler resulting in a corresponding axial movement of the coupler within the housing, wherein axial movement of the coupler in the proximal direction results in a corresponding axial movement of the outer jacket and delivery capsule in the proximal direction removing the delivery capsule from the prosthetic device.

Example 357: The flexible delivery catheter according to any example herein, particularly example 356, wherein the prosthetic device is coupled to a device catheter extending through a central lumen of the outer jacket, wherein the device catheter is movable within the central lumen of the outer jacket.

Example 358: The flexible delivery catheter according to any example herein, particularly example 357, wherein, during axial movement of the outer jacket in the proximal direction, the device catheter maintains the axial location of the prosthetic device such that the delivery capsule moves in a proximal direction removing the delivery capsule from the prosthetic device.

Example 359: The flexible delivery catheter according to any example herein, particularly examples 355-358, wherein the pullwire mechanism is engaged and the pullwire is moved in a proximal direction during axial movement of the outer jacket in the proximal direction to remove the delivery capsule from the prosthetic device.

Example 360: The flexible delivery catheter according to any example herein, particularly examples 329-359, wherein the inner member includes a longitudinally extending window extending through a sidewall of the inner member, wherein the proximal end portion of the coupler including the threaded outer surface extends through the window.

Example 361: The flexible delivery catheter according to any example herein, particularly examples 329-360, wherein the inner member includes more than one longitudinally extending windows (for example, two windows), extending through a sidewall of the inner member, wherein the proximal end portion of the coupler including the threaded outer surface of extends through each of the more than one longitudinally extending windows.

Example 362: The flexible delivery catheter according to any example herein, particularly examples 360-361, wherein the window extends along a majority of a total length of the inner member.

Example 363: The flexible delivery catheter according to any example herein, particularly examples 360-362, wherein a circumferential width of the window is greater than a circumferential width of the threaded outer surface of the coupler.

Example 364: The flexible delivery catheter according to any example herein, particularly examples 360-363, wherein a side wall surface of the proximal end portion of the coupler extending into and/or through the widow, slidingly engages a corresponding side wall surface of the window.

Example 365: The flexible delivery catheter according to any example herein, particularly examples 329-364, wherein a proximal end and a distal end of the housing coupled to the inner member.

Example 366: The flexible delivery catheter according to any example herein, particularly example 365, wherein a distal end of the inner member includes a housing coupling portion including a shoulder for engaging a corresponding shoulder provided on an inner surface of the housing, wherein engagement between the housing shoulder and the inner member shoulder limits axial movement of the inner member in a distal direction.

Example 367: The flexible delivery catheter according to any example herein, particularly examples 365-366, wherein a proximal end of the inner member includes a proximal housing coupling portion including a shoulder for engaging at least one of a shoulder and a distal end surface of the housing, wherein engagement between the inner member shoulder and at least one of the housing shoulder and the distal end surface of the housing limits axial movement of the inner member in a proximal direction.

Example 368: The flexible delivery catheter according to any example herein, particularly examples 329-367, further including an o-ring provided between the inner member and the housing, proximate a distal end of the housing (for example, the o-ring providing a sealing engagement between the housing and the inner member, additionally/alternatively, the o-ring can provides a bearing surface between the housing and inner member to facilitate rotational movement therebetween).

Example 369: The flexible delivery catheter according to any example herein, particularly example 368, wherein the o-ring is provided in a recess extending circumferentially around an outer surface of the inner member proximate a distal end of the inner member.

Example 370: The flexible delivery catheter according to any example herein, particularly examples 329-369, further including a second o-ring provided between the inner member and the housing, proximate a proximal end of the housing.

Example 371: The flexible delivery catheter according to any example herein, particularly example 370, wherein the second o-ring is provided in a recess extending circumferentially around an outer surface of the inner member proximate a proximal end of the inner member.

Example 372: The flexible delivery catheter according to any example herein, particularly examples 329-371, wherein a proximal end portion of the inner member extends proximally through and beyond a proximal opening in the housing (for example, cylindrical portion extending proximally from the housing providing a handle or gripping surface for the user, additionally/alternatively, the proximal end portion can be used to couple the delivery catheter to other medical equipment/tools).

Although several examples of the invention have been disclosed in the foregoing specification, it is understood by those skilled in the art that many modifications and other examples of the invention will come to mind to which the invention pertains, having the benefit of the teaching presented in the foregoing description and associated drawings. It is thus understood that the invention is not limited to the specific examples disclosed hereinabove and that many modifications and other examples are intended to be included within the scope of the appended claims. Moreover, although specific terms are employed herein, as well as in the claims which follow, they are used only in a generic and descriptive sense and not for the purposes of limiting the described invention nor the claims which follow. We, therefore, claim as our invention all that comes within the scope and spirit of these claims.

1. A flexible delivery catheter comprising:
an outer jacket, having a proximal end, a distal end, an inner surface and an outer surface, and defining a central lumen extending longitudinally between the proximal end and the distal end;
a coil layer disposed at least partially within the central lumen of the outer jacket, the coil layer comprising a coil winding and having a proximal end, a distal end, an inner surface, and an outer surface, wherein the coil winding extends helically about a longitudinal axis and defines a central lumen between the proximal end and the distal end; and
a coupler provided at a distal end of the outer jacket, the coupler having a proximal end, a distal end, and outer surface and defining a central lumen extending longitudinally between the proximal end and the distal end,
wherein the outer jacket includes a first portion with a first inner diameter and a second portion with a second inner diameter, the first portion adjacent the proximal end of the outer jacket, the second portion adjacent the distal end of the outer jacket, the second inner diameter being greater than the first inner diameter,
wherein the outer jacket further includes a shoulder disposed between the first and second portions, and
wherein the coupler and the second portion of the outer jacket form a cavity, and the coil layer is disposed within the cavity such that the coil layer abuts the inner surface of the second portion of outer jacket.

2. The flexible delivery catheter of claim 1, further including a delivery capsule with a central lumen, wherein the coupler couples the delivery capsule to the outer jacket,
wherein the central lumen of the delivery capsule is sized and configured to receive a prosthetic heart valve in a crimped or non-expanded configuration therein,
wherein the coupler is fixedly coupled to at least one of the outer jacket or the delivery capsule.

3. The flexible delivery catheter of claim 1, where the central lumen of the coupler includes a shoulder,
wherein the distal end of the coil layer abuts the shoulder of the coupler securing the coil layer between the shoulder of the coupler and the shoulder of the outer jacket.

4. The flexible delivery catheter of claim 2, wherein the central lumen of the coupler adjacent the delivery capsule includes an increasing taper between smaller diameter central lumen of the coupler and a larger outer diameter central lumen of the delivery capsule,
wherein the central lumen of the coupler includes a cylindrical portion adjacent the proximal end, an increasing taper portion adjacent the distal end, and a radially extending protrusion between the cylindrical portion and the increasing taper portion,
wherein a diameter of the radially extending protrusion is less than a diameter of the cylindrical portion,
wherein the coil layer is disposed in the cavity such that the proximal end of the coil layer abuts the shoulder of the outer jacket, and the distal end of the coil layer abuts a radially extending protrusion of the coupler.

5. The flexible delivery catheter of claim 1, further comprising at least one tensile stiffening fiber for limiting bending of the delivery catheter in a direction opposite a circumferential location of the at least one tensile stiffening fiber.

6. The flexible delivery catheter of claim 5, further including a braided layer disposed along at least a portion of the outer jacket,
wherein the at least one tensile stiffening fiber is provided along the braided layer,
wherein the at least one tensile stiffening fiber is not woven into a first portion of the braided layer,
wherein the at least one fiber is woven into a second portion of the braided layer, and
wherein the second portion of the braided layer is provided at a distal tip portion of the delivery catheter such that the tensile stiffening fiber is not woven into the braided layer at a location corresponding to the coil layer.

7. A flexible delivery catheter comprising:
an outer jacket, having a proximal end, a distal end, an inner surface and an outer surface, and defining a central lumen extending longitudinally between the proximal end and the distal end;
a hypotube disposed at least partially within the central lumen of the outer jacket, the hypotube having a proximal end, a distal end, an inner surface, an outer surface, and a plurality of cut-outs which extend between the inner surface and the outer surface of the hypotube such that the hypotube can bend omnidirectionally; and
a coupler provided at a distal end of the outer jacket, the coupler having a proximal end, a distal end, an outer surface, and defining a central lumen extending longitudinally between the proximal end and the distal end,
wherein the outer jacket includes a first portion with a first inner diameter and a second portion with a second inner diameter, first portion adjacent the proximal end of the outer jacket, the second portion adjacent the distal end of the outer jacket, and the second inner diameter being greater than the first inner diameter,
wherein the outer jacket further includes a shoulder disposed between the first and second portions, and
wherein the coupler and the inner surface of the outer jacket form a cavity, the hypotube is disposed within the cavity such that the hypotube abuts the inner surface of the second portion of the outer jacket.

8. The flexible delivery catheter of claim 7, wherein the hypotube is disposed in the cavity such that the proximal end of the hypotube abuts the shoulder of the outer jacket, and the distal end of the hypotube abuts a radially extending protrusion of the coupler.

9. The flexible delivery catheter of claim 7, further including a delivery capsule with a central lumen, where the coupler couples the delivery capsule to the outer jacket,
wherein the central lumen of the delivery capsule is sized and configured to receive a prosthetic heart valve in a crimped or non-expanded configuration therein,
wherein the coupler is fixedly coupled to at least one of the outer jacket or the delivery capsule.

10. The flexible delivery catheter of claim 7, wherein the central lumen of the coupler adjacent the delivery capsule includes an increasing taper between smaller diameter central lumen of the coupling segment and a larger outer diameter central lumen of the delivery capsule,
wherein the central lumen of the coupler includes a cylindrical portion adjacent the proximal end, an increasing taper portion adjacent the distal end, and a radially extending protrusion between the cylindrical portion and the increasing taper portion,
wherein a diameter of the radially extending protrusion is less than a diameter of the cylindrical portion.

11. The flexible delivery catheter of claim 7, wherein the central lumen of the coupler includes at least one axially extending recess,
wherein the distal end of the hypotube further includes at least one axially extending protrusion such that the at least one axially extending protrusion can be disposed within the at least on axially extending recess, and
wherein the central lumen of the coupler includes a shoulder and wherein the axial extending recess is provided in the shoulder such that when the axially extending protrusion is disposed within the axially extending recess, the hypotube is fixedly coupled to the coupler.

12. The flexible delivery catheter of claim 7, further including:
a braided layer disposed along at least a portion of the outer jacket; and
at least one tensile stiffening fiber for limiting bending of the delivery catheter in a direction opposite a circumferential location of the at least one tensile stiffening fiber, where the at least one tensile stiffening fiber is located at a neutral axis of the delivery catheter and provided along the braided layer, where the at least one tensile stiffening fiber is not woven into a first portion of the braided layer, and woven into a second portion of the braided layer,
wherein the second portion of the braided layer is provided at a distal tip portion of the delivery catheter such that the at least one tensile stiffening fiber is not woven into the braided layer at a location corresponding to the hypotube.

13. The flexible delivery catheter of claim 7, further including a ring having a proximal end, a distal end, and an outer surface and defining a central lumen, wherein the ring is disposed within the cavity such that the proximal end of the hypotube abuts the distal end of the ring,
wherein the distal end of the ring defines at least one axially extending recess,
wherein the proximal end of the hypotube further includes at least one axially extending protrusion such that the at least one axially extending protrusion can be disposed within the at least one axially extending recess,
wherein the distal end of the ring defines at least one axially extending projection,
wherein the proximal end of the hypotube further includes at least one axially extending recess such that the at least one axially extending protrusion can be disposed within the at least one axially extending recess.

14. A flexible delivery catheter comprising:
an outer jacket, having a proximal end, a distal end, an inner surface and an outer surface, and defining a central lumen extending longitudinally between the proximal end and the distal end, the outer jacket defining a flexible portion extending along a length of the outer jacket proximal the distal end;
a pullwire mechanism coupled to the outer jacket for directing bending motion of the flexible portion of the outer jacket, the pullwire mechanism including:
a pullwire extending along the outer jacket and coupled to the outer jacket at a location proximate the distal end of the outer jacket; and
a knob coupled to the pullwire such that rotational movement of the knob causes the pullwire to move in a proximal or distal direction resulting in a corresponding bending motion of the flexible portion of the outer jacket; and
a valve deployment assembly coupled to the outer jacket for directing proximal and distal movement of the outer jacket, the valve deployment assembly including:
a housing with a central lumen extending therethrough, the central lumen including a threaded inner surface;
an inner member extending within the central lumen of the housing, the inner member rotatably coupled to the housing and including a central lumen extending therethrough;
a coupler fixedly coupled to the outer jacket and rotatably coupled to the housing, the coupler extending within the central lumen of the housing and the inner member, a proximal end portion of the coupler including a threaded outer surface that threadingly engages the threaded inner surface of the housing;
wherein rotational movement of the housing causes the threaded inner surface of the housing to engage the threaded outer surface of the coupler resulting in a corresponding axial movement of the coupler within the housing and corresponding axial movement of the outer jacket in the proximal or distal direction.

15. The flexible delivery catheter of claim 14, wherein movement of the pullwire in a proximal direction, causes the distal end of the outer jacket to curve in a direction transverse to a longitudinal axis of the delivery catheter,
wherein movement of the pullwire in the distal direction, causes the outer jacket to move back toward the longitudinal axis of the delivery catheter.

16. The flexible delivery catheter of claim 14, wherein the pullwire mechanism further includes a threaded sleeve operatively coupling the knob and the coupler, the threaded sleeve including a central lumen extending therethrough and a threaded outer surface, the threaded sleeve positioned on a distal end portion of the coupler such that the threaded sleeve is axially movably along the coupler, the threaded outer surface of the threaded sleeve engaging a threaded inner surface of the knob,
wherein rotational movement of the knob causes the threaded outer surface to engage the threaded inner surface of the knob resulting in a corresponding axial movement of the threaded sleeve along the coupler.

17. The flexible delivery catheter of claim 16, wherein the pullwire mechanism further includes an anchor sleeve positioned on and axially movable along the distal end portion of the coupler between the threaded sleeve and a main body portion of the coupler,
wherein the pullwire is fixedly coupled to the anchor sleeve,
wherein rotational movement of the knob causes the threaded sleeve to engage the threaded inner surface of the knob resulting in a corresponding axial movement of the threaded sleeve and the anchor sleeve along the coupler, resulting in a corresponding movement of the pullwire in the proximal or distal direction.

18. The flexible delivery catheter of claim 14, wherein the coupler includes an alignment feature extending longitudinally along an outer surface of the distal end portion, the alignment feature engages a corresponding alignment feature provided on an inner surface of at least one of the threaded sleeve and the anchor sleeve,
wherein the engagement between the coupler alignment feature and at least one of the threaded sleeve alignment feature and the anchor sleeve alignment feature fixes the circumferential position of the threaded sleeve and/or anchor sleeve with respect to the coupler.

19. The flexible delivery catheter of claim 14, further including a delivery capsule coupled to the distal of the outer jacket,
wherein a delivery capsule coupler couples the delivery capsule to the outer jacket, the delivery capsule coupler is provided at a distal end of the outer jacket, the delivery capsule coupler having a proximal end, a distal end, and outer surface and defining a central lumen extending longitudinally between the proximal end and the distal end,
wherein a central lumen of the delivery capsule is sized and configured to receive a prosthetic device in a crimped or non-expanded configuration therein.

20. The flexible delivery catheter of claim 19, wherein rotational movement of the housing causes the threaded inner surface of the housing to engage the threaded outer surface of the coupler resulting in a corresponding axial movement of the coupler within the housing,
wherein axial movement of the coupler in the proximal direction results in a corresponding axial movement of the outer jacket and delivery capsule in the proximal direction removing the delivery capsule from the prosthetic device.
